(19) United States
(12) Patent Application Publication
(10) Pub. No.: US 2009/0156982 A1
(43) Pub. Date: Jun. 18, 2009

(54) Transdermal treatment device and method

(76) Inventors: Arezou Petrie, Tarzana, CA (US); Louis Petrie, Tarzana, CA (US); Xavier Roy, Tarzana, CA (US)

Correspondence Address: Colin P. Abrahams, Suite 400, 5850 Canoga Avenue, Woodland Hills, CA, 91367, US

(21) Appl. No.: 12/231,798

(22) Filed: Sep. 5, 2008

Related U.S. Application Data
(60) Provisional application No. 60/967,766, filed on Sep. 6, 2007.

(51) Int. Cl. A61M 35/00; A61K 31/5513; A61K 9/00; A61K 9/70; A61K 9/127; A61K 39/08

(52) U.S. Cl. 604/22; 514/221; 424/400; 424/449; 424/450; 424/236.1; 604/290; 604/310

(57) ABSTRACT

A treatment device for treating a medical condition comprises a composition selected for its ability to treat the medical condition, the composition being formulated for transdermal delivery. A portable container is provided which comprises a body for accommodating the composition in a sealed arrangement, a frangible seal on the container which can be ruptured to enable discharge of the composition from the body, and an application component on the container for facilitating transdermal delivery of the composition.

### CROSS REFERENCE TO RELATED APPLICATIONS

This application claims the benefit of U.S. Provisional Patent Application No. 60/967,766 filed on Sep. 6, 2007, and which is incorporated herein by reference in its entirety.

### FIELD AND BACKGROUND OF THE INVENTION

This invention relates to a composition and device for treating certain conditions such as seizures or other medical occurrences or conditions. Particularly, the invention is for a composition and device which, especially in combination, can be easily carried by a person, or the caregiver of a person, who suffers from the periodic onset or attack of a condition such as, for example, a seizure and which can be applied quickly and simply to effectively treat the condition.

In this specification, the term “medical condition” should be broadly interpreted to include any health related matter, elective or otherwise, regardless of its seriousness and may include the use of non-prescription creams, lotions or other compositions.

One important condition which can be treated in accordance with the present invention is, as mentioned, a seizure. Typically, people prone to seizures have these attacks periodically, but it is almost impossible to predict when a seizure will commence or what environmental stimuli may trigger a seizure. What is most important, however, is that the seizure be treated as soon as possible to reduce or minimize the deleterious effects thereof and the present invention provides a mechanism for effective treatment almost instantaneously and without regard to location. It is not necessary in accordance with the present invention for the effective treatment that appropriate medication be provided in a manner which requires a trained health professional, such as through injection or other invasive medical procedures which may embarrass the person receiving the medication.

While the invention may constitute a particularly effective mechanism for the treatment of seizures, it must be appreciated that the invention can be used in a large number of different treatments and medical conditions, some of which will be mentioned herein. Generally, the principles of the invention will be described with respect to representative medical conditions with the understanding that the invention can be widely applied in many contexts. Attached to this specification is Table I which although providing a fairly detailed list of possible applications for the present invention is not intended to be exhaustive or definitive.

### SUMMARY OF THE INVENTION

In one aspect, the invention relates to a formulated composition having an active ingredient selected for its ability to treat a medical condition. The active ingredient is preferably in a form which is best suited to the treatment in accordance with the invention.

In another aspect, the invention is for a device which is configured for the effective and relatively simple delivery of the composition in accordance with the invention, preferably a delivery which is topical and delivered to a part of the body which is readily accessible in normal conditions. The delivery device of the invention is designed for easy transportation and ready availability, as well as for simple and effective administration to the patient in a manner which can be performed by untrained individuals.

The device of the invention may also include protective components, such as a protective sheath, for use by a person applying the composition in order to protect the person from absorbing the composition on the hands as it is applied. The protective component may take many forms, such as a latex sheath for covering the finger, the hand or other configuration.

According to one aspect of the invention, there is provided a treatment device for treating a medical condition, the device comprising: a composition selected for its ability to treat the medical condition, the composition being formulated for transdermal delivery; and a portable container comprising a body for accommodating the composition in a sealed arrangement, a frangible seal on the container which can be ruptured to enable discharge of the composition from the body, and an application component on the container for facilitating transdermal delivery of the composition.

In one form, the composition may be in gel form, such as for example Diestat® gel.

In one of many embodiments, the composition is diazepam. In other embodiments, the composition may be selected from one of the following: Botox®, Viagra®, Cialis®, Levitra®, Nicotine, and Glycolic Acid. Furthermore, the composition may be selected from one of the generic name substances or drugs as set forth in Table I which is attached to this description.

In one embodiment, the portable container comprises a finger ring. The portable container may also comprise a necklace, a bracelet, or a bandage, to name a few examples only.

Preferably, the application component on the container comprises a vibrator. In one form, the vibrator is a vibrating capsule have a power source and switch for activating the vibrator. The vibrator, when moved over the composition during or after application thereof to the skin of the person requiring the treatment, assists in the transdermal delivery of the composition into the blood stream of the person thereby maximizing the effect of the composition in the shortest possible time.

Preferably, the finger ring comprises a band portion worn around the finger, a chamber for accommodating the composition, and a chamber for a vibrator. The device may also comprise a latex sheath, contained in a chamber or space in the ring, the latex sheath to be worn by a person facilitating transdermal delivery to prevent any portion of the composition from adversely affecting the person facilitating transdermal delivery, as mentioned.

Preferably, the composition comprises an active ingredient for treating the medical condition, the active ingredient being present as a dosage of about 0.05 mg/ml-50 mg/ml. In one embodiment, the active ingredient is present in the amount of about 0.5 mg/ml-30 mg/ml, and an optimal dosage may be when the active ingredient is present in the amount of about 5 mg/ml-20 mg/ml.

Preferably, the composition further comprises liposome cream as a carrier and a penetration enhancer, and may also comprise DMSO (Dimethyl Sulfoxide), which may be present in the amount of about 0.05% to about 90% as an example only, for skin penetration enhancement.

According to another aspect of the invention, there is provided a method of treating a medical condition comprising: providing a composition selected for its ability to treat the medical condition, and formulating the composition for transdermal delivery; placing the composition in a sealed chamber of a portable container; rupturing a frangible seal of the chamber when the composition is needed for treatment to enable discharge of the composition from the body; applying the composition to an accessible part of a person being treated with the composition; and facilitating the application of the composition to the person by utilizing an application component formed on the container to enhance transdermal delivery of the composition to the person.

Although the composition may be applied to many parts of the body, one particularly effective location of application is the area behind or below the ear, especially for anti-psychotic drugs. Not only is this area easily accessible at almost all times, but it is also an area where transdermal delivery may be carried out effectively. Furthermore, the proximity of this area to the brain is also of great import especially of the condition being treated is a seizure and the composition needs to reach the blood flowing to the brain as soon as possible to initiate the effects of the composition and stop the seizure.

Preferably, the application of the composition is facilitated by a vibrator to improve rapid transdermal delivery of the composition to the person. The vibrator speeds up absorption of the composition in the skin and the blood stream and may be part of a device kit for the treatment method of the invention. Furthermore, the method contemplates the use of a latex sheath, which may also be a part of the device kit, to protect the person applying the composition by preventing absorption of the medication by such person's fingers.

The method, as mentioned above, may be configured to facilitate the application of the composition to the person being treated behind or below the ear or temple of the treated person, especially when anti-psychotic drugs are being applied.

### BRIEF DESCRIPTION OF THE DRAWINGS

FIG. 1 is a schematic view of a person's head showing certain areas where the composition in accordance with the invention may be applied;

FIG. 2 is a schematic view of a ring in accordance with the invention which contains the composition in a sealed manner until needed, and also contains a vibrator and latex sheath;

FIG. 3 shows schematically a series of bandages of different shapes which may be used in applying the composition in accordance with the invention.

### DETAILED DESCRIPTION OF THE INVENTION

The present invention is a novel formulation of one of many different possible compositions, one of which may be diazepam is (sold under the brand name Valium®), that can be applied to the skin to abort or arrest a medical condition, a seizure attack in the case where diazepam is used, within minutes. The constituents of this cream allow the medication to penetrate the skin rapidly. It is then absorbed by the blood circulation and transported to the brain where is able to halt or slow the seizure event (for diazepam) within 2-3 minutes of its initial application. Because of the calming effects of diazepam, the cream has a secondary role as an easy to apply sedative, e.g. in medical procedures where anxiety or excessive movement can interfere with the test.

In this specification, the use of diazepam will often be used as an example of the invention, but the invention should not in any way be construed as being limited to this composition.

This composition of the invention, when an anti-seizure cream, has been effectively used on an epilepsy patient, a 5′ 10″, 275 pound, 19 year old man with mental retardation and intractable epilepsy. Despite three different anti-epileptic drugs ingested and prescribed in the conventional manner, he was still having weekly breakthrough seizures. The application of the cream in accordance with the invention, has prevented these seizures from lasting more than a few minutes.

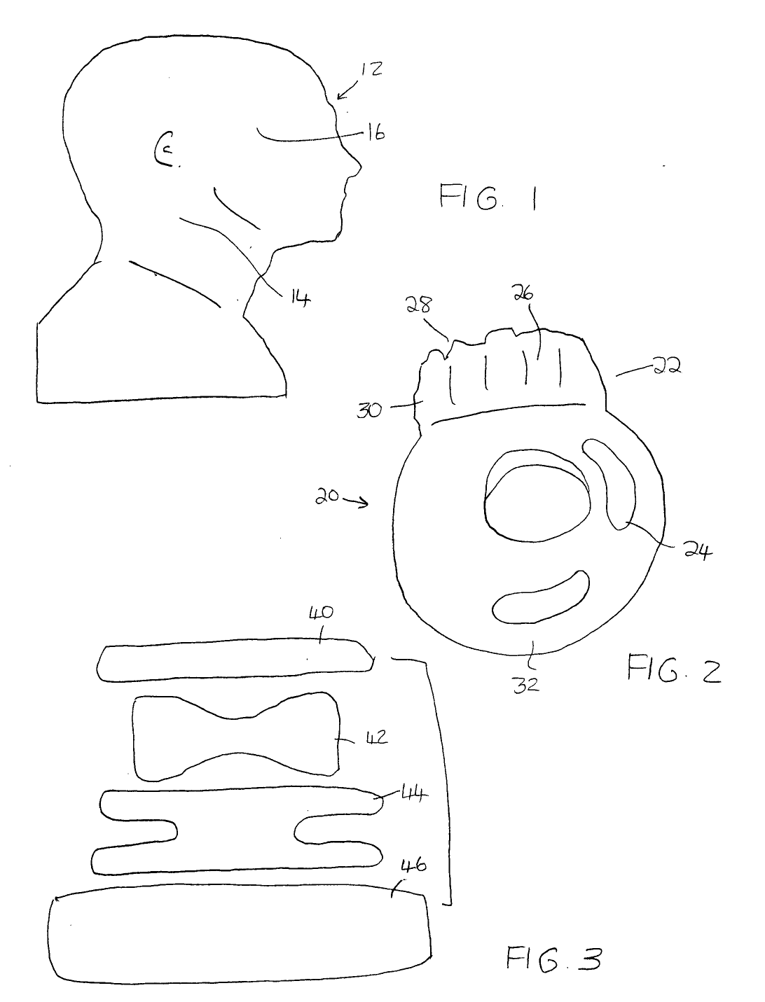

FIG. 1 of the drawings illustrates schematically the profile of a head 12 of a person showing possible advantageous sites for applying the composition of the invention. The composition may be applied to the ear region 14 or the temple region 16. These areas are good selections for the application when the composition is needed to get to the brain quickly, such as in a case of seizure.

FIG. 2 of the drawings shows a ring 20 in accordance with one aspect of the invention. The ring 20 comprises a housing 22 and a band portion 24. The housing 22 defines a chamber 26 for accommodating the composition, the chamber 26 being sealed. Preferably, the chamber 26 houses a specifically dosed amount of the composition. The chamber 26 can be opened or exposed by removal of a cover 28 so that the composition can be discharged to the desired surface of the body. A separate chamber 28 may contain a vibrator 30 which can be activated to facilitate penetration of the composition transdermally. Alternately, a single chamber may contain both the composition as well as the vibrator. In one form, the band portion 24 may be hollow and contain a latex sheath 32 which can be removed and used by the person applying the composition to the patient to prevent the person from contacting the composition, which may be harmful or undesirable.

FIG. 3 of the drawings shows four differently shaped bandages 40, 42, 44 and 46, each having a shape which may be appropriate for different parts of the body. The bandages contain the desired composition, and may also contain a vibrator or other mechanism for helping transdermal absorption of the composition. A latex sheath may also be provided as part of a bandage kit.

### Diazepam

In the emergency room, diazepam is the drug of choice to stop prolonged seizures. It is generally given intravenously and can be administered only by trained medical personnel. A major advance in seizure management came in 1995 when a rectal form of diazepam, Diastat®, became available. The drug could be given at home by parents or caregivers to stop prolonged or frequent seizures. By allowing earlier and effective treatment, this new formulation was an important step in giving parents greater control and reassurance over their children's seizure disorder. A parent can stop a seizure at home within a relatively short period of time, such as about 15 to 20 minutes, even before an emergency medical crew would arrive, thus preventing the potential of further brain injury and morbidity. It currently appears to be the only FDA approved medication for seizures that can be administered by lay people.

Diastat® comes in a gel form that must be given by syringe rectally. Furthermore, the buttocks of the patient must be held together for several minutes to ensure that all of the Diastat® is absorbed and not expelled prematurely from an unexpected bowel movement.

Important as it is, Diastat® suffers from several limitations. First, when administered to a child, the child must be repositioned and the pants and underwear pulled down. Second, the child must be briefly restrained for at least as long as the injection is administered. Third, the buttocks must be held together after administration. At home, this is of course simpler to achieve than when a child may be at school, at the local mall or on the road. However, as the child grows into adolescence and beyond, such maneuvers are more difficult to perform due to the size and weight of the child. (The example patient described above is a case in point.) It will be appreciated that application to a seizing adult in this manner would be difficult or even impossible. Note that Diastat® is indicated as a treatment for people of all ages and not just for children.

A topical diazepam anti-seizure cream which is delivered to the patient transdermally in accordance with the invention allows ease and reliable administration concurrently with the rapid onset of action, such as a seizure which attacks without warning. The cream is applied, in accordance with one aspect of the invention, to the mastoid area behind the ear on one side. The anti-seizure effect of the transdermal application of the composition can often occur within minutes of application. Advantageously, no significant repositioning or restraint of the patient is required. No removal of clothing is necessary which could embarrass the patient. It is, in fact, as simple and straightforward as applying sun tan lotion.

Chemistry and formulation of diazepam: A benzodiazepine, diazepam is a white to yellow, practically odorless crystalline powder with a melting point between 131-135° C. and pKa of 3.4. Diazepam is tasteless initially, but a bitter after-taste develops. One gram is soluble in 333 ml of water, 25 ml of alcohol, and it is sparingly soluble in propylene glycol. The pH of the commercially prepared injectable solution is adjusted with benzoic acid/sodium benzoate to 6.2-6.9. It consists of a 5 mg/ml solution with 40% propylene glycol, 10% ethanol, 5% sodium benzoate/benzoic acid buffer, and 1.5% benzyl alcohol as a preservative.

Pharmacology of diazepam: The subcortical levels (primarily limbic, thalamic, and hypothalamic) of the central nervous system are depressed by diazepam and other benzodiazepines thus producing the anxiolytic, sedative, skeletal muscle relaxant, and anti-convulsant effects seen. The exact mechanism of action is unknown, but postulated mechanisms include: antagonism of serotonin, increased release of and/or facilitation of gamma-aminobutyric acid (GABA) activity, and diminished release or turnover of acetylcholine in the central nervous system. Benzodiazepine specific receptors have been located in the mammalian brain, kidney, liver, lung, and heart. In all species studied, receptors are lacking in the white matter.

Diazepam is used clinically for its anxiolytic, muscle relaxant, hypnotic, appetite stimulant, and anti-convulsant activities.

Diazepam is rapidly absorbed following oral administration. Peak plasma levels occur within 30 minutes to 2 hours after oral dosing. The drug is slowly (slower than oral) and incompletely absorbed following IV administration.

Diazepam is highly lipid soluble and is widely distributed throughout the body. It readily crosses the blood-brain barrier and is fairly highly bound to plasma proteins.

Diazepam is metabolized in the liver to several metabolites, including desmethyldiazepam (nordiazepam), temazepam, and oxazepam, all of which are pharmacologically active. These are eventually conjugated with glucuronide and eliminated primarily in the urine. Because of the active metabolites, serum values of diazepam are not useful in predicting efficacy.

Human-Approved Products including diazepam are Diazepam oral tablets (2 mg, 5 mg, 10 mg); Diazepam timed-release oral capsules 15 mg; Diazepam oral solution 1 mg/ml in 500 ml containers and unit-dose (5 & 10 mg) 5 mg/ml in 30 ml dropper bottle; and Diazepam Injection 5 mg/ml in 2 ml amps & syringes & 1.2, 10 ml vials, 2 ml Tel-E-Ject; Emulsified Injection: 5 mg/ml in 3 ml vials.

### Formulation

One aspect of the present invention is the medication configuration and formulation, as well as its base. When diazepam or similar or other types of medications are mixed into a “liposome” base, and applied behind the ear or at the temporal lobe area, the rate of absorption of the medication is rapid. This can be used to advantage, for example, in status epilepsy, anxiety disorder or psychotic break-outs. Application of the cream is not an invasive procedure and can be performed by any care provider. It lacks the danger of injections, and does not cause embarrassment of suppository application or discomfort of a nose drop or spray. It may also prevent “cheeking” of the medication (meaning that the patient cannot easily keep the pill in the cheeks and dispose of it when unobserved, thereby assuring that medication is taken) or choking from a swallowed medication, a highly relevant problem especially during a seizure. Finally, external application of this agent may prevent or reduce the adverse effects commonly seen in these medications. Applying the medication transdermally may prevent the “first pass” effect which is elimination of the drug through the liver. The same is true of the clearance of the drug through the kidney. This is know as reverse kinetics.

As regards nasal sprays, potential complications arising from nasal spray delivery include accessability, rupture of the nostrils, and the possibility of aspiration.

The present invention also has the ability to modify the time of absorption by using a different base.

Overall, therefore, the present invention in one form thereof addresses a new line of medication delivery, which may be specifically suited and adapted for the medical condition being treated.

Yet a further aspect of the present invention addresses further the delivery of the medication. Current patients using the medication must typically squirt out a dose thereof from a large container. One aspect of the present invention details how the medication may be placed in various devices such as a ring worn device. The unique advantage to this form of delivery is it defines and doses a specific unit of cream or composition needed for a recognized potential medical occurrence, makes application thereof easy for anyone regardless of training, and may prevent undesirable medication absorption to the person administering the medication. In accordance with one aspect of the invention, there is also addressed the inclusion of a vibrating device to enhance penetration of the medication, as described above.

The present invention thus preferably relates to two distinct and independent parts. The first one concerns the field of drug delivery, particularly to the topical administration of pharmaceutical drugs (example, anti-epileptic agents, anti-psychotic agents) suspended in a rapidly absorbed cream, and applied to select areas of the body (example, behind the ear or temples). The second one concerns the use of various delivery devices that may vibrate or otherwise be configured so that penetration of the cream is enhanced during and/or after the application of the cream (example, a vibrating finger-ring applicator), and that includes an optional drug dispenser unit that may or may not automatic. Pharmaceuticals applied using this process of the present invention and contained in these unique and medically effective devices will enter the body faster with quicker clinical effects.

When diazepam is the drug of choice for treating seizure, the diazepam is suspended in a unique cream base. The cream can be formulated to either release drug rapidly once administered, or over time. The short-acting cream may be formulated using a liposomal forming component. The long-acting cream may use a PLO (Pluronic Lecithin Organogel) base. Upon seizure activities, an exact amount of cream is applied to the neck, below the ear or other area selected for efficient transmittal. Data from one patient tested showed an onset of action of the medication of about three minutes. This can be favorably compared to 15 to 30 minutes for the onset of action from a rectally-administered diazepam product.

One example of the delivery solutions for pharmaceuticals provided in accordance with the invention is a capsule-like device attached to or contained within a space or chamber within a ring. Within the capsule which is full of the medicated cream there can also be a small vibrator. The ring is packaged as a single unit of use making it easy for a patient to carry, easy to wear and easy for medication application, especially in an emergency. In addition, the ring protects the wearer from contact with the medication by properly isolating the medication in a safe and effective manner, and this can be especially important if a different person to the patient is applying the drug. The vibration of the cream on the surface of the skin will improve blood flow to the site of application and improve the absorption of the drug into the skin. The drug delivery device would be smaller for the wearing on the finger and may include, for example, a latex sheathing to cover the ringed finger or other body part against any exposure to the pharmaceutical medication. An upper capsule would be similar in size and may contain a small vibrator. The capsule may also contain micro-sized openings so that once a protective layer or cover is removed, pressure would push out or discharge the medication from the capsule to the desired area.

A similar effect, process and method would be applicable to our unique bandage device.

### Examples of Formulation

In accordance with one aspect of the invention, all compositions discussed below can be interchanged by any drug of the same family. For example, any benzodiazepins, such as Ativan, Lorazepam, klonopin (clonazepam) or compositions not limited to any benzodiazepine family and their combinations may be used.

The drug of choice can be phenobarbital not limited to barbiturates and any combination of thereof with one another and benzodiazepines plus Progesterone or estrogen or a combination of all or one based on patient respond.

For all the ingredients below, in order to achieve long acting effects the base can be replaced to PLO gel instead of liposome cream and the use of DMSO is discretionary. The PLO gel can be applied through a patch or any other similar device to achieve slower delivery of the drug and to maximize and enhance slower penetration of the drug as best suited to the needs and particulars of the patient.

A higher dose of medication may satiate the cream and cake and as a result not be effective. The ranges given below provide broad ranges to cover the preferred choices.

Comfort Cream—immediate relief for seizure (statues epileptic-us)

Active ingredient: Diazepam (Valium®)

Range of active ingredient:

| Broad | Intermediate | Optimal |
|---|---|---|
| 0.05 mg/ml-50-mg/ml | 0.5 mg/ml-30 mg/ml | 5 mg/ml-20 mg/ml |

Other Essential ingredient:

Liposome cream to qs per recipe (drug vehicle and penetration enhancer).

Other Non essential ingredients:

DMSO (Dimethyl Sulfoxide) 0.05% to 90% use for (skin penetration enhancement)

Ethyl alcohol 0.5% to 10% (use for solvent and diluent)

Seizure and Migraine Cream

Application and usage: To apply the appropriate amount of the cream and massage (depending on appropriate dosage by individualized dosing based on specifics patients), apply behind the two ears and on and around the temples; the estimated time to stop seizures may be under 3 minutes, noting equivalent and as convenient in the market for seizure treatment. Effective time for treatment of migraine headaches may be under 5 minutes.

For Seizure (Statues Epileptic-Us) and Migraine

Range

Active ingredient: Diazepam (Valium®)

| Broad | Intermediate | Optimal |
|---|---|---|
| 0.05 mg/ml-50-mg/ml | 0.5 mg/ml-30 mg/ml | 5 mg/ml-20 mg/ml |

Active ingredient: Progesterone

| Broad | Intermediate | Optimal |
|---|---|---|
| 0.05 mg/ml-200 mg/ml | 5 mg/ml-150 mg/ml | 20 mg/ml-100 mg/ml |

Essential ingredient:

Liposome cream to qs per recipe (drug vehicle and penetration enhancer)

Non essential ingredients:

DMSO (Dimethyl Sulfoxide) 0.05% to 90% use for skin penetration enhancement;

Ethyl alcohol 0.5% to 10% use for solvent and diluent)

Application and usage: To apply appropriate amount of the cream and massage (depending on appropriate dosage by individualized dosing based on specifics patients), apply behind the two ears and on and around the temples; estimated time to stop seizures under 3 minutes noting equivalent and as convenient in the market for seizure treatment. It may work in under 5 minutes for migraine headaches.

### For Seizure (Statues Epileptic-Us) and Migraine

Range

Active ingredient: Diazepam (Valium®)

| Broad | Intermediate | Optimal |
|---|---|---|
| 0.05 mg/ml-50-mg/ml | 0.5 mg/ml-30 mg/ml | 5 mg/ml-20 mg/ml |

Active ingredient: Progesterone

| Broad | Intermediate | Optimal |
|---|---|---|
| 0.05 mg/ml-200 mg/ml | 5 mg/ml-150 mg/ml | 20 mg/ml-100 mg/ml |

Active ingredient: Lidocain

Essential ingredient:

Liposome cream to qs per recipe (drug vehicle and penetration enhancer)

Non essential ingredients:

DMSO (Dimethyl Sulfoxide) 0.05% to 90% use for skin penetration enhancement;

Ethyl alcohol 0.5% to 10% use for solvent and diluent)

Application and usage are typically as described above.

### Vibrating and Dispensing Applicators

Any cream, liquid or fluid (referred to hereafter as substance) when applied manually on the body of humans or animals require most of the time some rubbing to facilitate the penetration through the body and to locate the exact point of application for a more effective action. The present invention describes and discloses the penetration of the substance and the most effective body locations to apply the substance. Any vibrating device that would include a substance dispenser and design as a ring or as a hand piece may be used that would allow the dispensing of the substance while massaging the effective location.

Ring: An applicator aid made of a soft and elastomeric material comprises a tubular body and a ring, wherein a hollow space is disposed inside the tubular body and has an opening formed where the vibration and dosing units will be inserted. The ring may be adapted for any finger.

The inside diameter of the ring may be between 1/12 of an inch to 1 inch. Preferably, the front end of said tubular body is thickened. One or more posts may be disposed on the outer surface of the tubular body. The soft elastomeric material may be plastic rubber, silicon rubber or latex. In one embodiment, a vibrator or light-emitting diode capsule is inserted into a hollow space of the tubular body through the opening. The tubular body may be a cylindrical body, a conical body, or a sphere-like body.

In one form, a dispensing or dosing unit is inserted into the hollow space of the tubular body through the opening. The tubular body may have a diameter of 1/10 of an inch and up to 20 inches.

Instead of a ring, the device may be a bracelet, a necklace or a keychain.

The present invention thus relates to an application aid, especially to an applicator aid having a ring holding firmly on the base of any finger and a hollow cone body which may be positioned behind an ear lobe for contacting or vibrating this part of the body to enhance transdermal delivery of the cream, liquid or fluid which has an antipsychotic, relaxing, and calming effect, acting on the brain. It would be positioned on the top of any organ on which the cream, liquid or fluid are known to have an effect.

Sometimes when applying the cream, liquid or fluid on someone, it is important to avoid any unintended contact with the cream, liquid or fluid. This is the case for example when applying medicated cream which could have an effect on the person applying as the cream is rubbed into the patient. Furthermore some of these creams have to be applied with a strict dosage while an accelerated penetration would enhance their effect.

In one embodiment, there is provided an applicator aid made of soft and elastomeric material comprising a tubular body wherein a hollow space is disposed inside the tubular body and has an opening formed where the vibration and dosing units will be inserted. The tubular body has a size adapted for any hand.

In another form, the invention provides an applicator aid made of any bandage which has a vibrating and dosing unit as part of it or attached to it in any manner. The transformed band aid has a size and shape which may be adapted for part of the body. The front end of the band aid may be thickened. Several posts may be disposed on the outer surface of the band aid.

An active and sensitive location of effective application of the composition is behind the ear lobe for any cream, liquid or fluid that has an effect on the brain. Other locations may comprise on or around the temples, the base of the neck, the top of the head through the hair on the soft part of the skull, or on the forehead in the middle of the two eyebrows that has an effect on the brain. Or, the cream may be applied on the skin in the area of any organ that has an effect on this organ.

TABLE I
Based Cream Product Recipes):

| | GENERIC NAME/Brand Name | BROAD RANGE | INTERMEDIATE | OPTIMAL RANGE | AMOUNT |
|---|---|---|---|---|---|
| A | Acacia - Acacia nilotica | 0.05 mg/ml-50 mg/ml | 0.5 mg/ml-30 mg/ml | 5 mg/ml-20 mg/ml | |
| | BASE CREAM OR GEL | | | | |
| B1 | Liposome cream (for fast acting results) | 1% to 70% | 7% to 50% | 10% to 30% | QS to 30 gm |
| B2 | Vanicream ® (for intermediate-timed results) | 1% to 70% | 7% to 50% | 10% to 30% | QS to 30 gm |
| B3 | VanPen ® (for good overall absorption) | 1% to 70% | 7% to 50% | 10% to 30% | QS to 30 gm |
| B4 | PLO Gel (for long-acting results) | 1% to 70% | 7% to 50% | 10% to 30% | QS to 30 gm |
| | DISCRETIONARY INGREDIENTS | | | | |
| C1 | (Skin penetrant enhancement - optional) | | | | |
| C2 | DMSO (Dimethyl Sulfoxide) | 0.05% to 50% | 0.1% to 30% | 1% to 5% | |
| | NON-ESSENTIAL INGREDIENTS | | | | |
| D1 | Ethyl Alcohol | 0.5% to 15% | | | |
| D2 | Glycerin | 0.5% to 15% | | | |

| | |
|---|---|
| E | MEDICATION USED FOR: *premature ejaculation * cure for rabies |

Based Lollipop Product Recipes:

| | GENERIC NAME/Brand Name | BROAD RANGE | INTERMEDIATE RANGE | OPTIMAL |
|---|---|---|---|---|
| A | Serotonin HCL | 0.05 mg/ml-200 mg/ml | 0.5 mg/ml-100 mg/ml | 5 mg/ml- |
| | BASE LOLLIPOP | | | |
| B | Sorbitol | 0.05% to 90% | 0.5% to 90% | 0.5% to |
| | NON-ESSENTIAL INGREDIENTS | | | |
| C | Flavoring | 0.05% to 15% | 0.5% to 25% | 0.5% to |

| | |
|---|---|
| E | MEDICATION USED FOR: Depression |

All the following GENERIC NAMES here under can replace the GENERIC NAME rows A in table 1 and 2 here above to create a new cream or
lollipop recipe while the “USED FOR” here under would replace row E in table 1 and table 2 here above. When the GENERIC NAMES hereunder
include a different “BROAD RANGE, INTERNEDIATE AND OPTIMAL” replace the full row A in table 1 and table 2 by the full rows A here under
and replace the “USED FOR” hereunder in row E here above. For all the rest of the rows (B1, B2, B3, B4, C1, C2, D1, D2, B, C) in table 1 and
table 2 will stay the as above. The herbal/plant/tree recipes here under are claimed for any member of the same genus even if only one member of
the genus is mentioned, in all combination of any members of any genus mentioned hereafter, and in any form of any genus' member such as but not
limited to leaves, stem, branch, plant, flower, fruit, and root. Each of the recipe composed with this formula is a claim.

HERBAL TYPE

| GENERIC | |
|---|---|
| NAME | USED FOR: |
| Acacia - Acacia | *premature ejaculation * cure for rabies |
| nilotica | |
| Adhatoda vasica - | Blood Conditions * bleeding due to idiopathic (unknown) thrombocytopenic (blood does not have enough platelets) purpura |
| Adhatoda vasica | (excessive bruising) Cardiovascular Conditions * moderate hypotensive activity (lowers blood pressure) * pulmonary diseases |
| | Female Conditions * assists uterine involution (rolling or turning inward) * menorrhagia (excessive menstrual bleeding) * |
| | postpartum (after childbirth) hemorrhage * uterine stimulant activity Gastrointestinal Conditions * dyspepsia * local bleeding |
| | due to peptic ulcer and/or piles (hemorrhoids) Respiratory Tract Conditions * acute and chronic bronchitis often combined |
| | with the herbs Licorice and Marshmallow for soothing the respiratory tract * allergic asthma, often combined with the herbs |
| | Albizia, Baical Skullcap. Eyebright and Tylophora * antihistamine effects (the leaves may be dried and smoked to relieve |
| | asthma) * broncho-dilation effects * emphysema * obstructive airway disease (chronic) often combined with the herbs |
| | Polygala, Grindelia, Euphorbia and Elecampane * phthisis (wasting of the body as in tuberculosis) * relieves cough and |
| | breathlessness Other Conditions * bleeding gums (applied locally) * diphtheria * gives relief in pyorrhoea, a gum disease |
| | (applied locally) * intermittent fever * Tuberculosis * typhus fever |
| Agrimony - | Blood Conditions * anemia Cardiovascular Conditions * enlargement of heart Gastrointestinal Conditions * diarrhea, |
| Agrimonia | especially mild, nonspecific, acute diarrhea - help control loose stools * digestive trouble * enlargement of stomach * |
| eupatoria | indigestion * mucous colitis * overactive irritable bowel * promotes assimilation of food * relaxed bowels * sluggish stomach |
| | conditions * stimulates the flow of digestive juices * stimulates the appetite * ulcers Genitourinary Conditions * kidney and |
| | bladder disorders * urinary tract infections combined with Hawthorn, Golden Rod and Thyme Inflammatory Conditions * |
| | gout * rheumatism Liver Conditions * facilitates liver secretion * gallbladder problems * hardening of liver * jaundice and |
| | other liver ailments * obstruction of the liver * sluggish liver conditions Respiratory Tract Conditions * coughs * enlargement |
| | of lungs * inflammation of oral and pharyngeal mucosa * inflammation of the throat - reduce inflammation and relieve sore |
| | throat pain * tonsillitis Mental Conditions * conditions of mental torture and worry * hiding behind a cheery facade when |
| | feeling mentally challenged * laryngitis * public speakers * singers * throat disorders * thrush or inflammation of the mucous |
| | membranes of the mouth * tonsillitis Skin Conditions It is useful internally and externally for skin problems, skin eruptions |
| | and diseases of the blood such as: * blotches * pimples * scrofulous sores *reduce the severity of certain types of skin |
| | inflammation, such as: * inflamed hemorrhoids, * oozing wounds * rashes * varicose veins * athlete's foot (fomentation) * |
| | drawing out splinters, thorns, etc. * gout * insect bites or stings * mild, superficial inflammation of the skin * scrofula * slow- |
| | healing wounds * sores on lower legs * ulcers * varicose veins * Other Conditions * appendicitis * fevers * lumbago * spleen |
| | disorders * tones the system |
| Albizia - Albizia | Allergic Conditions * reduces the release of histamines through a stabilizing effect on mast cells * mildly suppresses activity |
| lebbeck | of T-lymphocytes reducing the level of allergy-inducing antibodies Blood Conditions * high blood cholesterol Respiratory |
| | Tract Conditions * asthma * allergic rhinitis (commonly called hay fever) is an inflammation or irritation of the mucous |
| | membranes that line the nose. Skin Conditions * eczema (internal and external) * urticaria (nettle rash) |
| Alfalfa - | relieve fluid retention and swelling * treat kidney stones Blood Conditions * anemia (with Nettles and Oatstraw) * blood |
| Medicago sativa | thinner * boils * helps the blood clot Brain and Nervous System Conditions * recuperation of narcotic and alcohol addiction |
| | Cardiovascular Conditions * heart disease * prevents the formation of atherosclerotic plaques * stroke Female Conditions * |
| | late or irregular menstruation * menopausal symptoms * stimulates lactation (milk supply) in nursing mothers Fungal |
| | Conditions * anti-fungal Gastrointestinal Conditions * appetite stimulant for loss of appetite * bowel problems * digestive |
| | disorders * dyspepsia * peptic ulcers * poor assimilation of nutrients * poor digestion * regulates the bowels * relieves gastric |
| | ulcers * stomach upset Genitourinary Conditions * eliminates retained water * fluid retention and swelling * kidney, bladder |
| | and prostrate disorders * kidney cleanser * kidney stones * relieves urinary problems * urinary tract infections Glandular |
| | Conditions * nourishes pituitary gland * promotes pituitary gland function Immune System Conditions * hay fever |
| | Inflammatory Conditions * arthritis Liver Conditions * blocks absorption of cholesterol * detoxifies the the liver * jaundice |
| | Male Conditions * prostrate disorders Respiratory Tract Conditions * asthma Other Conditions * alkalizes and detoxifies the |
| | body * cancer (binds carcinogens in the colon to help speed up their elimination from the body) * debility * diabetes * overall |
| | tonic for general health * fatigue * stimulates the growth of supportive connective tissue * weight gain * boils * insect bites |
| All Spice - | colds * menstrual cramps * upset stomach * diabetes * flatulence * indigestion * refreshing tonic * bruises * joint and muscle |
| Pimenta | pains Brain and Nervous System Conditions * hysterical conditions Gastrointestinal Conditions The dried herb is used for: * |
| officinalis | diarrhea * dyspepsia * flatulence (gas/wind) * gripe * flatulent indigestion (1 or 2 drops on sugar) * gripe Inflammatoty |
| | Conditions Externally local anesthetic effect * neuralgia * rheumatism Muscular Conditions * alleviates muscle pain Other |
| | Conditions * Soothe and relieve toothaches |
| Aloe Vera - Aloe | Bacterial, Viral, Fungal and Parasitic Conditions * AIDS * eliminates parasites (especially protozoan infections) * relieves |
| barbadensis | Herpes simplex & zoster * relieves symptoms of Candida * relieves symptoms of Epstein-Barr virus (chronic fatigue |
| | syndrome) * relieves symptoms of fungal infections * relieves symptoms of genital herpes * relieves symptoms of yeast |
| | infections * Staphylococcus infections * vaginitis * viral infections Blood Conditions * aids hyperglycemia * aids in blood |
| | circulation * hemorrhoids * lowers blood sugar in people with type 2 (non-insulin-dependent) diabetes Cardiovascular |
| | Conditions * relieves hypertension Female Conditions * menstrual cramps and irregularity * suppressed menes |
| | Gastrointestinal Conditions * acts mainly on the large intestine * colic * constipation * dyspepsia * facilitates digestion by |
| | aiding the immune system * helps cleanse the digestive tract by exerting a soothing, balancing effect * helpful to the stomach, |
| | small intestine and colon * intestinal infection * keeps the bowels functioning smoothly and helps when there is an impaction |
| | * moves allergenic proteins from the small intestine into the colon * aids in healing wounds by drawing out infections and |
| | preventing infection from starting * naturally alkalizes digestive juices to prevent over-acidity * promotes a favorable balance |
| | of gastrointestinal symbiotic bacteria and decreased yeast populations * relieves nausea * relieves symptoms of indigestion * |
| | stimulates activity of the gastrointestinal tract * stimulates intestinal motility Genitourinary Conditions * assists kidney |
| | function Immune System Conditions * allergic reactions * has immune-stimulating actions * strengthens the body's natural |
| | resistance Inflammatory Conditions * relieves arthritis Liver Conditions * assists liver and gall bladder functions * helps |
| | hypercholesterolemia Respiratory Tract Conditions * colds * relieves symptoms of esophagal reflex * relieves symptoms of |
| | gingivitis Other Conditions * aids in lymphatic circulation * as a cathartic for persons of a phelgmatic nature or sedentary |
| | habits * helps pruritis * is beneficial for persons of a sedentary nature or phlegmatic constitution * ulcerations |
| Amaranth - | Blood Conditions * hemorrhages, especially bowel * spitting of blood Female Conditions * menorrhagia (excessive |
| Amaranthus spp., | menstruation) Gastrointestinal Conditions * diarrhea * dysentery * gastroenteritis * stomach influenza Respiratory Tract |
| Amaranthus | Conditions * ulcerated conditions of the throat and mouth * sores and ulcers * be used for tick bites * reduce tissue swelling |
| hypochondriacus | from sprains * leucorrhea |
| Andrographis | Cardiovascular Conditions * cardiovascular disease Immune System Conditions * acute and chronic infections * weakened |
| Paniculata | immunity Liver Conditions * toxic liver damage * liver infections * poor liver function Other Conditions * malaria |
| | (traditionally considered to be superior to quinine in malaria) * worm infestation |
| Angelica - | Brain and Nervous System Conditions * nervous headache Cardiovascular Conditions * poor circulation * promotes |
| Angelica | circulation and energy in the body * stimulates and warms the circulation Female Conditions * expels afterbirth * painful |
| archangelica | menstruation * stimulates circulation in the pelvic region * stimulates suppressed menstruation Gastrointestinal Conditions * |
| | colic, especially in children * digestive difficulties * eases flatulence (gas/wind) * eases indigestion * heartburn * stimulates |
| | and warms the digestive system * stimulates appetite * stimulates digestion * stomach and intestinal difficulties * stomach |
| | cramps with vomiting * stomach ulcers * strengthens a weak stomach Genitourinary Conditions * ailments of the urinary |
| | organs * gout * stimulates kidney action Immune System Conditions * protects against disease Inflammatory Conditions * |
| | rheumatism Liver Conditions * hepatitis * liver problems Respiratory Tract Conditions * ailments of the respiratory system * |
| | breathing problems * chronic bronchitis * cold and damp in lung disease * loosens phlegm in the lungs * pleurisy * relieves |
| | symptoms of colds * soothes coughs * sore throat * stimulates and warms the lungs Other * fevers, especially intermittent * |
| | general debility * muscle spasms * relieves pain * eyes * gout * lung and chest diseases * nerves * relieving rheumatic pains * |
| Anise - | Bacterial Conditions * eradicates intestinal bacteria Brain and Nervous System Conditions * epilepsy * insomnia Female |
| Pimpinella | Conditions * induces menstruation * promotes milk in nursing mothers Gastrointestinal Conditions * digestive infections * |
| anisum | colic, especially in infants * eases colic-type pains * flatulence (gas) * halitosis (bad breath) * improves appetite * indigestion |
| | * nausea * promotes digestion * purifies intestines, with Caraway and Fennel * soothes digestion * expels flatulence * is used |
| | as a stomach tonic * relieves cramps and spasms Inflammatory Conditions * infantile catarrh Respiratory Tract Conditions * |
| | bronchitis * chest infections * loosens tight phlegm * persistent hard, dry coughs * reduces congestion and irritation in the |
| | lungs * spasmodic asthma Other * alleviates cramps * lice * scabies * the eyes (diluted) |
| Apricot - prunus | * Cancer * ulcer *aphrodisiac * skin cleansing * heart disease * reduce “bad cholesterol” levels * regenerating body fluids |
| armeniaca | 8detoxifying * quenching thirst * toning the respiratory system * alleviating a cough |
| Astragalus - | acterial or Viral Conditions * Candida * chronic bacterial infections, especially if combined with debility and spontaneous |
| Astragalus | sweating * Chronic Fatigue Syndrome * chronic hepatitis * chronic viral infections, especially if combined with debility and |
| membranaceous | spontaneous sweating, e.g. AIDS * prevention and treatment of the common cold Cardiovascular System Conditions * |
| | congestive heart failure * high blood pressure * lowers blood pressure * relieves symptoms and improves heart function in |
| | severe forms of heart disease Blood Conditions * anemia Female Conditions * prolapsed uterus * uterine bleeding |
| | Gastrointestinal Conditions * general digestive disorders including * bloating * diarrhea * gas (flatulence) * peptic ulcers * |
| | stomach ulcers * strengthens digestion Genitourinary Conditions * nephritis * promotes the secretion of urine Immune |
| | Conditions * aids in strengthening the immune system * chronic autoimmune diseases * chronic impaired immunity * fortifies |
| | battered immune systems of people undergoing cancer chemotherapy or radiation * gives the immune system a powerful |
| | boost * helps the body to fight off viral and bacterial infections responsible for causing colds, flu, bronchitis and sinus |
| | infections * immune enhancing properties, especially in chronic hepatitis * multiple allergies * persistent infection * |
| | prevention of infection * speeds recovery and extends life expectancy for people whose immune systems have been |
| | compromised by chemotherapy or radiation Metabolic Conditions * raises metabolism * strengthens metabolismratory |
| | Conditions * chronic phlegm production * shortness of breath Other Conditions * builds stamina * fatigue or lack of appetite |
| | associated with chemotherapy treatment * fever * heals wounds and injuries * helps to counteract cell damage caused by |
| | unstable oxygen molecules called free radicals * improves overall health * increases endurance * night sweats * organ |
| | prolapsus * promotes healing and tissue regeneratio * raises vitality * stops debilitating sweating |
| Baical Skullcap - | Allergic Conditions * allergic and inflammatory diseases * allergic rhinitis * hay fever * inhibits prostaglandin production and |
| Scutellaria | histamine release Blood Conditions inhibits collagen and arachidonic acid induced platelet aggregation Cardiovascular |
| baicalensis | Conditions * atherosclerosis * hypertension Gastrointestinal Conditions * bacillary dysentery Immune system Conditions * |
| | acute infections * autoimmune diseases * chronic infection * retroviruses, e.g. herpes, HIV etc. Inflammatory Conditions * |
| | chronic inflammation Liver Conditions * cholecystitis * hepatitis Metabolic Conditions * cataract and diabetes complications |
| | Pregnancy * abnormal fetal movements * toxemia in pregnancy Respiratory Conditions * acute bronchitis * asthma * colds |
| | Other * clears heat and damp heat * leukemia * scarlet fever * tumors * acne vulgaris * eczema * peridontal disease * |
| | urticaria |
| Barberry - | Brain and Nervous System Conditions * produces a sense of well-being * promotes vigor Cardiovascular Conditions * dilates |
| Berberis vulgaris | blood vessels * high blood pressure Gastrointestinal Conditions * constipation (larger doses) * diarrhea * dyspepsia * |
| | heartburn * improves appetite * regulates digestion * relieves upset stomach Inflammatory Conditions Berries: * |
| | inflammatory fevers, like typhoid Liver Conditions * biliary disorders * biliousness * functional derangement of the liver * |
| | gallbladder disease * jaundice * liver complaints * secretes bile Respiratory Tract Conditions It is used for: * mouth and |
| | throat irritation * relieving pyorrhea * sore throat * strengthening the gums Other Conditions * debility in general * |
| | intermittent fevers * reduces fever * spleen problems Berries: * scurvy * cutaneous eruptions |
| Basil - Ocymum | Brain and Nervous System Conditions * clears the head and brain * headache (rubbed on the temples) * mild nervous |
| basilicum, | disorders Female Conditions * cleanses the womb * expels and afterbirth after childbirth Gastrointestinal Conditions * |
| Ocymum | constipation * enteritis * gastric catarrh * intestinal catarrh * stomach cramps * vomiting Respiratory Tract Conditions * colds |
| minimum | * whooping couth Other * draws out poisons |
| Bay Laurel - | Brain and Nervous System Conditions * hysteria Female Conditions * amenorrhea * brings about a speedy delivery during |
| Laurus nobilis | childbirth * eases childbirth * expels afterbirth * induces menses (periods) Gastrointestinal Conditions * flatulent colic * |
| | stimulates appetite * stimulates digestive system Inflammatory Conditions * rheumatism Respiratory Tract Conditions * colds |
| | * coughs * lung disorders * chest problems * colds Other * removes obstructions * bruises * earache * soothing itchy skin * |
| | rheumatism- * skin problems * sprains |
| Bayberry - | Female Conditions * leucorrhoea * uterine hemorrhage Gastrointestinal Conditions * canker of the stomach and bowels * |
| Myrica cerifera, | diarrhea * dysentery * internal ulcers Glandular Conditions * scrofula * Liver Conditions * jaundice Respiratory Tract |
| Myrica | Conditions * sore or inflamed throat * tonsillitis * internal antiseptic [an agent for inhibiting the growth of microorganism on |
| communis | living tissue or destroying pathogenic or putrefactive bacteria] * improves circulation by increasing the vitality of all the |
| | systems of your body and is therefore beneficial for: * chills * clamminess * coldness of the extremities * vaginal infections * |
| | on varicose veins to strengthen the blood vessels combined with Slippery Elm * for old ulcers * to beautify the skin |
| Bergamot - | colds * diarrhea * flatulent colic * influenza * nausea * rheumatism * urinary disorders * vomiting * acne * coughs * |
| Monarda | depression * fevers * stress * tension * as a hair wash for dandruff * for rheumatism |
| citriodora, | |
| Monarda didyma, | |
| Monarda | |
| fistulosa, | |
| Monarda punctata | |
| Bilberry - | Blood Conditions * blood disorders * prevents capillary fragility * reduces clotting * stops bleeding * thins the blood * |
| Vaccinium | thrombosis * varicose veins Brain and Nervous System Conditions * improves blood supply to the nervous system |
| myrtillus | Cardiovascular Conditions * angina * atherosclerosis (plaque in blood vessels that leads to blockage, heart attacks and |
| | strokes) * helps to reduce the risk of blood clot which may leads to heart attacks and strokes * improves circulation * lowers |
| | blood pressure * prevents the oxidation of LDL (“bad”) cholesterol * stimulates the release of vasodilators * strengthen blood |
| | vessels, * vascular disorders Female Conditions * dries up milk during weaning Genitourinary Conditions * dropsy * gravel * |
| | urinary complaints Immune System Conditions * infectious diseases Metabolic Conditions * diabetes - type 2 (adult onset) |
| | diabetes (lowers blood sugar) Parasitic Conditions * expels parasites Gastrointestinal Conditions * derangements of the bowel |
| | * diarrhea (reduces the intestinal inflammation associated with this condition) * dysentery * intestinal putrefaction (which |
| | produces flatulence/gas/wind) * regulates bowel action * soothes the digestive tract * stimulates appetite * stomach problems |
| | * stomach ulcers (may help prevent stomach ulcers caused by stress, medications, alcohol etc.) Eye Conditions * improves |
| | visual accuracy in healthy people * natural antioxidant preventing free radical damage to the eye * poor vision and ‘night |
| | blindness’ * improves the microcirculation and regeneration of retinal purple, a substance required for good eyesight * |
| | protects eyes from eyestrain or fatigue * cataracts * glaucoma * macular degeneration * myopia * nearsightedness * |
| | pigmentosa * retinitis (Bilberry may help protect the retina, the nerve layer lining the back of the eye that sends nerve |
| | impulses to the visual areas of the brain) Other Conditions * discharges * scurvy * ulceration of mouth and throat * typhoid |
| | fever |
| Bistort - | Bacterial Conditions * cholera * measles Blood Conditions * all bleedings internal and external * hemorrhoids * lung |
| Polygonum | hemorrhages * nose bleeds * stomach hemorrhages Gastrointestinal Conditions * bowel complains * diarrhea, especially with |
| bistorta | blood in the stools * dysentery Genitourinary Tract Conditions * incontinence Immune System Conditions * infectious |
| | diseases Liver Conditions * jaundice * bruises * loose teeth * nasal polyps * stops bleeding in cuts and wounds * worms in |
| | children (powdered leaves) * all gum problems * hardening spongy gums * looseness of teeth * sore mouth * sore throat * |
| | stomatitis * bites from venomous creatures * discharging ulcers * hemorrhages * running sores Other * diabetes (combined |
| | with other tonics) * intermittent fever * mucous discharges, especially from ears, nose, vagina, urethra |
| Black Cardamom - | *treat stomach disorders *malaria |
| Amomum | |
| costatum or | |
| subulatum | |
| Black Cohosh - | Brain and Nervous System Conditions * a sedative effect on the nervous system * chorea (St. Vitus' Dance) especially in |
| Cimicifuga | children * hysteria * relieves depression Cardiovascular Conditions * a cardiac stimulant * improves blood circulation * |
| racemosa | reduces rapidity of pulse Female Conditions * contracts the uterus * delayed and painful menstruation * menopause |
| | symptoms (usually taken in conjunction with other herbs) including * hot flushes * irritability * mood swings * sleep |
| | disturbances * menstrual cramps * obstructed menstruation * painful menstruation * premenstrual discomfort * promotes and |
| | restores healthy menstrual activity * soothes irritation and congestion of the female reproductive system * vaginal atrophy |
| | Gastrointestinal Conditions * diarrhea in children (in small doses) Inflammatory Conditions * arthritis * helps relieve |
| | symptoms of osteoarthritis, combined with Willow Bark (Salix alba.), Sarsaparilla (Smilax spp.), Guaiacum Resin (Guaiacum |
| | officinale) and Poplar Bark (Populus tremuloides) * reduces inflammation associated with osteoarthritis and rheumatoid |
| | arthritis * rheumatism Respiratory Tract Conditions * chronic bronchitis * consumption * eases coughs * spasmodic problems |
| | such as whooping cough Skeletal Conditions * may help prevent osteoporosis Other Conditions * induces perspiration * |
| | wasting diseases |
| Blackberry - | Cardiovascular Conditions * weak circulation Female Conditions * leucorrhea Gastrointestinal Conditions * diarrhea, |
| Rubus fructicosus | especially summer diarrhea * dysentery * enteritis Genitourinary Conditions * dropsy Respiratory Conditions * whooping |
| | cough in the spasm stage Other * chronic appendicitis * fever Other Uses: * burns * scalds * bleeding gums |
| Bladderwrack - | Brain and Nervous System Conditions * sustains the nervous system and the brain Female Conditions * excellent boost for |
| Fucus vesiculosis | pregnancy and the developing fetus Genitourinary Conditions * influences the kidneys Glandular Conditions * goiter * |
| | obesity (particularly associated with an under-active thyroid gland) * scrofula * strengthens and promotes the glands * |
| | stimulates activity in the thyroid gland Inflammatory Conditions * arthritis * rheumatism Metabolic Conditions * regulates |
| | metabolism Other * diseases of the hips, joints and bones in children * arthritis * enlarged or hardened glands * rheumatism * |
| | burns * scalds * bleeding gums |
| Blessed Thistle - | Brain and Nervous System Conditions * dizziness * hysteria * melancholia * migraine headache * nervous prostration * |
| Cnicus | strengthens the brain and the memory * vertigo Cardiovascular Conditions * purification and circulation of blood Female |
| benedictus | Conditions * promotes milk supply to nursing mothers Gastrointestinal Conditions * constipation * digestive problems * |
| | flatulence * a tonic * increasing appetite * weak and debilitated conditions of the stomach Liver Conditions * liver and |
| | gallbladder ailments Parasitic Conditions * worms * intermittent fever * chill blains * sores * wounds Other * general disease |
| | preventative * chronic headache * earache |
| Blue Cohosh - | Blood Conditions * dissolves fats in the bloodstream Brain and Nervous System Conditions * epilepsy * hysteria Female |
| Caulophyllum | Conditions * all female complaints * expedites a speedy delivery during childbirth, in very small doses combined with |
| thalictroides | Squawvine and Black Cohosh (do not start taking any earlier than two weeks before delivery date) * induces labor * regulates |
| | menstruation * suppressed menstruation * uterine inflammation Gastrointestinal Conditions * colic, especially in children * |
| | cramps, especially in children Genitourinary Conditions * dropsy Inflammatory Conditions * rheumatism |
| Boneset - | Female Conditions * uterus conditions Gastrointestinal Conditions * bowel conditions * stomach conditions Inflammatory |
| Eupatorium | Conditions * catarrhal conditions * muscular rheumatism Liver Conditions * liver conditions Respiratory Tract Conditions * |
| perfoliatum | colds in general * influenza Other * fever * severe intermittentfevers * skin diseases |
| Borage - Borago | Blood Conditions * purifies blood Brain and Nervous System Conditions * nerve tonic * nervous conditions Cardiovascular |
| officinalis | Conditions * palpitation of the heart * pulmonary complaints Female Conditions * promotes milk in nursing mothers |
| | Genitourinary Conditions * promotes the activity of the kidneys Glandular Conditions * activates the Adrenal Glands |
| | Inflammatory Conditions * catarrh * peritonitis * rheumatism Liver Conditions * jaundice Respiratory Tract Conditions * |
| | feverish colds * pleurisy * mouth ulcers * sore throat * inflammatory swellings Other * antidote to some poisons * reduces |
| | fevers * reduces high temperatures * restores vitality during convalescence from illness |
| Bramhi - Bacopa | Brain and Nervous System Conditions * brain tonic * epilepsy * insanity * improves memory * improves mental capacities, |
| monniera | such as concentration and learning * nerve tonic * nervous breakdown * nervous deficit due to injury and stroke * nervous |
| | exhaustion |
| Buchu - Barosma | Cardiovascular Conditions * high blood pressure Gastrointestinal Conditions * stomach problems Genitourinary Conditions It |
| betulina | exerts a direct effect on the urinary organs and is used for: * bladder infections * blood in the urine * chronic urinary tract |
| | disorders * cystitis * gravel and catarrh of the bladder * inflammation of the urethra * kidney stones * nephritis * painful |
| | urination * vaginal disorders [leucorrhea] Inflammatory Conditions * arthritis * rheumatism Male Conditions * certain |
| | prostate problems |
| Bupleurum - | Female Conditions * irregular menstruation * prolapse of the uterus Gastrointestinal Conditions * prolapse of the rectum * |
| Bupleurum | regulates gastrointestinal function Immune System Conditions * acute infections * autoimmune diseases involving the liver or |
| falcatum | kidneys Inflammatory Conditions * chronic inflammatory disorders Liver Conditions * acute and chronic liver disease * |
| | chemical liver damage * enlarged liver * poor liver function * restores liver function Respiratory Tract Conditions * chronic |
| | cough * common cold with chills and fever Other Conditions * debility * Often combined with Astragalus for debility and |
| | prolapse |
| Burdock - | Blood Conditions * clears toxins from the bloodstream Female Conditions * building the systems of young women |
| Arctium lappa | Gastrointestinal Conditions * constipation * digestive system ailments and complaints * stimulates the digestive organs * |
| | stomach ailments: * indigestion * strengthening and toning the stomach Genitourinary Conditions * bladder pain * fluid |
| | retention * may be helpful for kidney stones * promotes the excretion of urine * stimulates the eliminatory organs Glandular |
| | Conditions * swollen glands Inflammatory Conditions * arthritis * inflammations * gout * osteoarthritis * rheumatic |
| | conditions, characterized by: * joint discomfort * loss of mobility * rheumatism * rheumatoid arthritis Liver Conditions * |
| | hepatitis * jaundice * liver problems * stimulates secretion of bile * a very effective liver cleanser and stimulator [combined |
| | with Dandelion root] Metabolic Conditions * diabetes Respiratory Tract Conditions * asthma Skin Conditions * Burdock |
| | works well alone or combined with Yellow Dock and Sarsaparilla for skin conditions * acne * boils * contact dermatitis * |
| | eczema * promotes perspiration * psoriasis * relieves skin problems * scalp conditions * wounds Other Conditions * certain |
| | cancers * fevers * neutralizes and eliminates poisons from the system * sciatica * scurvy, caused by vitamin C deficiency, |
| | resulting in: * bleeding * gum disease * weakness * abscesses * acne * all manner of swellings * boils * catarrh * hair growth, |
| | especially with Blind Nettle * scaly skin conditions * syphilis * ulcers for gouty swellings for old sores and ulcers for |
| | shrinking arteries and sinews for tumors to relieve bruises to relieve inflamed surfaces |
| Butcher Broom - | Female Conditions * female obstructions Genitourinary Conditions * dropsy * gravel * nephritis * urinary obstructions Liver |
| Ruscus aculeatus | Conditions * jaundice Lymphatic Conditions * scrofulous tumors Respiratory Conditions * clears the chest of phlegm * |
| | relieves difficult breathing |
| Calendula - | Blood Conditions * abscesses * blood in urine * boils Cardiovascular Conditions * stimulates circulation Female Conditions * |
| Calendula | menstrual difficulties Gastrointestinal Conditions * colitis * diarrhea * gastric and duodenal ulcers * gastritis * gastro- |
| officinalis | intestinal disorders * inflammation of the large intestine * prevents recurrent vomiting * stomach cramps * stomach ulcers |
| | Genitourinary Conditions * dropsy Liver Conditions * jaundice * infectious hepatitis * liver disorders * stimulates the flow of |
| | bile Parasitic Conditions * worms and parasites Other * cancer * brings out bruises * cleanses * children's ailments * fever * |
| | headaches * helps to prevent internal complications if taken after an accident * improves the healing of wounds * visceral |
| | obstructions * abscesses * Athlete's foot * bedsores * bee or wasp stings * boils * bruises * burns * contusions * eczema * |
| | festering or cancer-like sores * fistulas * frost bites * fungus infestation * hemorrhoids * phlebitis * pigment spots * |
| | preventing suppuration * preventing the formation of scars or lessening old scars * psoriasis * pulled muscles * scalds * slow |
| | healing wounds * sores * sprains * strengthening eyes and red eyes * swellings * ulcers * warts * varicose ulcers * varicose |
| | veins |
| Californian | Brain and Nervous System Conditions * insomnia * nervousness * over-excitedness and sleeplessness in children |
| Poppy - | Gastrointestinal Conditions * colic pains Liver Conditions * gallbladder colic Respiratory Tract Conditions * chronic coughs |
| Eschscholzia | Other * pain |
| californica | |
| Caraway - Carum | Brain and Nervous System Conditions * hysterical disorders Female Conditions * promotes the onset of menstruation * |
| carvi | relieves uterine cramps * promoting the production of breast milk in nursing mothers Gastrointestinal Conditions * diarrhea * |
| | dyspepsia * flatulence (gas/wind) * flatulent colic in infants * settles the stomach after taking nauseous medicines * soothes |
| | the digestive system * easing colic when rubbed on the abdomen of the infant Respiratory Tract Conditions * bronchitis * |
| | easing chest congestion * boils in the ear * earache * toothache * for sprained ankles * to reduce bruising |
| Cascara - | Blood Conditions * hemorrhoids Gastrointestinal Conditions * acts principally on the large intestine * digestive complaints * |
| Rhamnus | dyspepsia * stimulant to the whole digestive system * habitual chronic constipation * improves bowel tone * intestinal gas * |
| purshiana | promotes gastric digestion * stimulates the appetite Liver Conditions * enlarged liver * liver and gall bladder complaints |
| | Other * suitable for delicate and elderly people |
| Catnip - Nepeta | Brain and Nervous System Conditions * mild sedative for children * hysteria * insanity * nervous headaches * nervousness * |
| cataria | nightmares * restlessness Female Conditions * infertility in women Gastrointestinal Conditions * colic * relieves pain of |
| | flatulence (gas/wind) Respiratory Tract Conditions * colds * coughs Other * children's ailments * pain relief * scarlet fever |
| | (combined with Saffron)acts upon the Sacral Plexus * it will relieve hemorrhoids * it will relieve painful swellings * it will |
| | reduce bruising * head sores * scurf |
| Cat's Claw - | Blood Conditions * protects small blood vessels of the brain from free radical damage Cardiovascular Conditions It may |
| Uncaria | reduce the risk of stroke and heart attack by: * controlling cholesterol * increasing circulation * lowering blood pressure * |
| tomentosa | reducing heart rate Female Conditions * female hormonal imbalances * Premenstrual Syndrome Gastrointestinal Conditions * |
| | acts on the digestive systems * colitis * Crohn's disease * diarrhea * diverticulitis * gastric inflammationgastric ulcers * |
| | intestinal ailments [often used homeopathically] * leaky bowel syndrome Genitourinary Tract Conditions * urinary tract |
| | infections Immune System Conditions * acts on the immune and digestive systems * boosts the body's immune system * |
| | diseases relating to immune system malfunction where inflammation occurs * helps the body fight off infections * improves |
| | its response to viral and respiratory infections * protects against degenerative diseases Any situation where immune system |
| | suppression is induced, such as: * bone-marrow transplants * organ transplants * skin grafts Inflammatory Conditions * |
| | arthritis * allergies * lupus Male Conditions * prostate conditions Metabolic Conditions * control of diabetes Parasitic and |
| | Viral Conditions * chronic fatigue syndrome * viral infections such as herpes and other retroviruses * parasites * treatment of |
| | AIDS [in combination with AZT] Respiratory Tract Conditions * allergic respiratory conditions * rheumatoid arthritis Other * |
| | skin impurities * tumors * healing of wounds |
| Cayenne - | Blood Conditions * stops internal bleeding making it excellent for use with ulcers Brain and Nervous System Conditions * |
| Capsicum spp., | depression Cardiovascular Conditions * benefits the heart and circulation when taken alone or added to other remedies * |
| Capsicum | circulation problems worsened by cold weather * equalizes the circulation * normalizes blood pressure * used as part of |
| minimum | treatment in alcoholism in relieving chronic congestion by reducing dilated blood vessels * warming and stimulating actions |
| | on the circulation * weakness or deficiency of the circulatory system Female Conditions * menstrual cramps Gastrointestinal |
| | Conditions * atony of the intestines and stomach * flatulence (gas/wind) * loss of appetite * sluggish dyspepsia * stimulates |
| | digestion * stomach and bowel cramps * stomach and bowel pains cramps * warming and stimulating actions on the digestion |
| | and circulation * weakness or deficiency of the digestive system Immune System Conditions * general stimulant * helps |
| | prevent disease in people exposed to cold and damp for any length of time * preventative to keep away disease * builds up |
| | resistance at the beginning of a cold Respiratory Tract Conditions * used for chills generally * respiratory problems associated |
| | with cold, damp weather Other * produces natural warmth * stimulates the action of other herbs * used in antibiotic |
| | combinations * is used for poor circulation (footbath) * is very effective added to liniments for all sorts of arthritis and muscle |
| | aches * will stop external bleeding * can be sprinkled [a small amount] * arthritis * pericarditis * pleuritis * rheumatism |
| Cedar - Thuja | Female Conditions * amenorrhea (absence of menstruation) * Genitourinary Conditions * dropsy * gout Respiratory Tract |
| occidentalis, | Conditions * chronic coughs * colds * shortness of breath Inflammatory Conditions * rheumatism Others * intermittent fevers |
| Thuja orientalis | * scurvy * rheumatism * venereal warts * fungal growths * muscular aches and pains * removing warts * rheumatism * skin |
| | afflictions |
| Celery Seed - | Brain and Nervous System Conditions * mild tranquilizer * antidote to alcoholism * anxiety * calms tense nerves * hysteria * |
| Apium | mental depression * nervous cases with lack of tone, combined with Skullcap * nervousness * neuralgia * promotes |
| graveolens | restfulness and sleep Cardiovascular Conditions * chronic pulmonary catarrh * lowers blood pressure in cases of high blood |
| | pressure Female Conditions * increases menstrual flow * promotes the onset of menstruation Gastrointestinal Conditions * |
| | flatulence (gas/wind) * indigestion * lack of appetite Genitourinary Conditions * acid-forming deposits * cystitis * gout * |
| | inflammation and/or infections of the urinary tract (Celery Seed's diuretic action combined with its anti-bacterial compounds |
| | make it a useful herb for treating urinary tract infections) * kidney infections * lowers the levels of uric acid in the body * |
| | promotes the excretion of urine * removes water from the body in the form of urine, which helps wash away bacteria and the |
| | minerals that cause kidney and bladder stones * stimulates the kidneys inducing increased urine flow * stimulates the removal |
| | of waste products from the body via the kidneys * strengthens the muscles in the uterus * urinary antiseptic Inflammatory |
| | Conditions Reduces inflammation in the following conditions: * arthritis * gout * rheumatism * rheumatoid arthritis Liver |
| | Conditions * liver problems * liver tonic * prevents liver damage caused by toxic chemicals Metabolic Conditions * corrects |
| | imbalances found in acid-based metabolisms Muscular Conditions * helps reduce muscle spasms * lumbago * promotes |
| | healthy muscular conditions Reproductive Conditions * aphrodisiac, with Damiana and Kola Nut * restores sexual potency |
| | impaired by illness Respiratory Tract Conditions * bronchitis * colds * influenza Other Conditions * clears up skin problems |
| | * deficiency diseases * prevention of cancer * prevents tumors * spleen disorders * swellings in body cavities * tendency |
| | towards overweight |
| Chamomile | Brain and Nervous Conditions * highly strung or hypersensitive people * infantile convulsions * insomnia * many nervous |
| (German) - | conditions * nerves * nerve sedative * neuralgia * paralyzed limbs * relaxes the nervous system * soothes anxiety and stress * |
| Matricaria | to calm the body * for inducing sleep in insomniac conditions Female Conditions * cessation of menstrual flow * cramps * |
| chamomilla | menstrual disorders * painful menstruation * for menstrual cramps Gastro-intestinal Conditions * a tonic upon the gastro- |
| | intestinal canal * diarrhea * digestive system affected by nervous tension * dyspepsia * facilitates bowel movement without |
| | acting directly as a purgative * flatulence * gastritis * griping pains * relaxes an overactive stomach * relieves indigestion * |
| | soothes gastric irritation * stimulates an under-active digestion * stomach aches * stomach disorders Genitourinary Conditions |
| | * dissolves kidney stones * inability to pass urine Inflammatory Conditions * reduces inflammation * rheumatic pains * |
| | rheumatic problems Male Conditions * inflammation of the testes Respiratory Tract Conditions allergic asthma alleviates |
| | symptoms of a cold sinus troubles strong congestion Other * disorders of the spleen * exhaustion after severe illness * fever * |
| | lumbago * tiredness of limbs * toothache * colds * infantile convulsions * stomach troubles * teething problems and for |
| | calming an overactive or over-excitable child * brightening the eyes * conjunctivitis * firming the tissues * inflamed eyes * |
| | inflammation of mucous tissue * keeping the skin young-looking * moist and itching skin eruptions * rashes * skin problems |
| | * wounds * for asthma in children * for irritation and inflammation in the respiratory tract * for sinusitis * to relieve |
| | symptoms of a cold * aching joints * hemorrhoids (Sitz Bath) * ill bladder (compress of flowers) * muscular aches * neuralgia |
| | * sweaty hands or feet (hand or foot bath) * weariness * for dental caries * for massaging overstrained or cramped muscles * |
| | for neuralgia * for pain in the ear canal * for pain of rheumatism * for paralyzed limbs * to firm the tissues * to keep the skin |
| | young-looking |
| Chamomile | Brain and Nervous System Conditions * delirium tremens due to alcoholism or drug withdrawal * hysterical and nervous |
| (Roman) - | people * nightmares * restlessness in children Gastrointestinal Conditions * colic * dyspepsia * flatulent colic * heartburn * |
| Anthemus nobilis | indigestion (used in combination with Ginger) * loss of appetite * sluggish intestines * spasms * stomach cramps * summer |
| | diarrhea in children Genitourinary Conditions * dropsy * gout Female Conditions * period headaches Other * tonic for the |
| | aged * fever in children * open sores * redness or inflammation of the skin * sunburn * wounds * congested neuralgia * |
| | earache * facial swelling from abscesses * inflammatory pain * intestinal inflammation * toothache * ease all over body pain |
| | * remove weariness * calluses * hard swellings * painful joints |
| Chaparral - | Bacterial, Parasitic and Viral Conditions * cold sores * parasites * venereal disease Blood Conditions * hemorrhoids |
| Larrea Mexicana | Gastrointestinal Conditions * heartburn * indigestion * intestinal cramps * stomach disorders Genitourinary Conditions * |
| | bladder and kidney problems * kidney problems Female Conditions * menstrual cramps * menstrual difficulties * regulates |
| | menstrual irregularities Inflammatory Conditions * rheumatism * rheumatoid arthritis Respiratory Tract Conditions * asthma |
| | (combined with Ginger and Ginkgo biloba) * colds Other * snake bites * wound healing * bites * bruises * cuts * disinfecting |
| | and deodorizing the body * hair and scalp diseases * menstrual cramps * sores |
| Chickweed - | Blood Conditions * blood builder Brain and Nervous System Conditions * palsy * paralysis Gastrointestinal Conditions * |
| Stellaria media | colitis * chronic constipation * coughs and hoarseness * flatulence (gas/wind) * inflammation of the bowels * inflammation |
| | or weakness of the digestive tract Genitourinary Conditions * kidney disorders Inflammatory Conditions * rheumatism |
| | Respiratory Tract Conditions * asthma * bronchitis * colds * coughs * hoarseness * inflammation of the lungs and bowels * |
| | internal consumption * pleurisy * relieves catarrh of the chest * relieves cramps Other * for under-nourished children to gain |
| | strength * obesity * peritonitis * scurvy * bruises * carbuncles * eczema * external abscesses * hemorrhoids * inflammation * |
| | itches * old ulcers * ophthalmia * psoriasis * reducing enlarged joints * rheumatism * skin diseases * skin rashes * skin ulcers |
| | * soothing and healing for damaged skin * soothing skin irritations * sores * swollen and inflamed tissue |
| Cinnamon - | Gastrointestinal Conditions * prevents uncomfortable bloating * relieves diarrhea * relieves flatulence (gas/wind) * relieves |
| Cinnamomum | griping * relieves nausea * relieves weakness of the stomach * stops vomiting Female Conditions * helps stop womb |
| zelanicum | hemorrhages * a sexual stimulant to the female Immune System Conditions * prevents infection |
| Cloves - Eugenia | helps the memory * insomnia, combined with mint and rose * melancholy, combined with mint and rose Gastrointestinal |
| aromatica | Conditions * clears the stomach of foul gases and putrefied matter * colic, combined with Allspice, Ginger and Cinnamon * |
| | dyspepsia * flatulence (gas/wind) * indigestion * nausea * purifies the abdominal passages * stimulating peristalsis * |
| | vomiting Parasitic Conditions * liver flukes, combined with Black Walnut Hulls and Wormwood * parasites Respiratory Tract |
| | Conditions * bronchial conditions Other * toothache * helping eyesight |
| Coleus forskohlii | Blood Conditions * anti-platelet action, combined with Ginger and Turmeric Cardiovascular Conditions * cerebrovascular |
| | disease (vasodilation), combined with Ginkgo biloba * congestive heart failure, combined with Craetagus, Astragalus, Panax |
| | Ginseng and/or Convallaria * hypertension (high blood pressure), combined with Craetagus * ischemic heart disease, |
| | combined with Craetagus and/or Salvia miltiorrhiza * thrombosis Gastrointestinal Conditions * digestive insufficiency * |
| | stimulates upper digestive function, combined with Gentian Glandular Conditions * hypothyroidism Eye Conditions * |
| | glaucoma (internally and externally) Respiratory Tract Conditions * asthma * chronic obstructive airway disease |
| | (bronchodilation) Reproductive Conditions * poor fertility Skin Conditions * psoriasis Other * weight loss |
| Coltsfoot - | Brain and Nervous System Conditions * giddiness with headache Gastrointestinal Conditions * diarrhea * nausea |
| Tussilago farfara | Genitourinary Conditions * stone formation Glandular Conditions * scrofula combined with Wormwood Respiratory Tract |
| | Conditions * a general cough remedy, especially combined with Horehound and Marshmallow * a racking, dry, unproductive |
| | cough * bronchial asthma * chronic bronchitis * damage caused by smoking * hoarseness * irritation and tightness of chest * |
| | laryngitis * persistent cough * pharyngitis * phlegm on the chest * pleurisy * pneumonia * shortness of breath * throat catarrh |
| | * tuberculosis (onset) * wheezing * asthma * bronchitis * catarrh * chest and breathing problems * coughs * erysipelas * |
| | bursitis * phlebitis * pneumonia * the swelling and discoloration of bruises * burns * general swellings * inflammations * |
| | insect bites * leg ulcers * swollen feet * chronic bronchitis * shortness of breath * earache |
| Comfrey - | Blood Conditions * anemia * bleeding from the lungs * hematuria (blood in the urine) * internal hemorrhages * spitting of |
| Symphytum | blood Cardiovascular Conditions * defective circulation * pulmonary complains Female Conditions * excessive menstrual |
| officinale | flow * leucorrhea Gastrointestinal Conditions * diarrhea * digestive and stomach problems * disorders of the digestive system |
| | * dysentery * intestinal difficulties * ulcers of the stomach and duodenum Inflammatory Conditions * calms inflammation * |
| | rheumatism Respiratory Tract Conditions * asthma * bronchitis * lung conditions * persistent coughs * tuberculosis * |
| | whooping cough * bleeding gums * hoarseness * throat inflammation Other * general tonic, with Alfalfa and Parsley * |
| | stimulates the growth of new cells, by speeding up the stage at which DNA is produced Externally it is used for: Blood |
| | Conditions * severe cuts * stops bleeding * varicose ulcers * varicose veins Female Conditions * sore, swollen breasts * |
| | leucorrhea Genitourinary Conditions * gout * relieves the pain of gout Inflammatory Conditions * any inflammation * |
| | relieves the pain of arthritis * relieves the pain of rheumatism Muscular System * bruises * contusions * joint pains * painful |
| | amputation stumps * pulled tendons and ligaments * sprains * swelling of joints Nervous System Conditions * paralyzed |
| | limbs, caused from over exertion, dislocation, sprain or shock * paraplegia Respiratory Conditions * bronchitis * pleurisy |
| | Skeletal System * broken bones * neck pains * slipped discs Skin Conditions * abscesses * acne * boils * burns * gangrenous |
| | ulcers * insect bites * obstinate ulcers * running sores * swellings * tissue damage * wound healing * regenerating aging skin |
| | Other * excessive mucous |
| Commiphora | Cardiovascular Conditions * ischemic heart disease Liver Conditions * high blood cholesterol and triglycerides Skin |
| mukul | Conditions * acne Other Conditions: * leprosy * obesity * rheumatism * syphilis |
| Cornflower - | Gastrointestinal Conditions * dyspepsia * eye weakness and inflammation * venomous bites |
| Centaurea cyanus | |
| Corydalis | Blood Conditions * blood stasis (stoppage of flow) Brain and Nervous System Conditions * insomnia * neuralgia |
| ambigua - | Cardiovascular Conditions * acute myocardial infarction * angina pectoris * cardiac arrhythmia * hypertension * myocardial |
| Corydalis | ischemia Female Conditions * dysmenhorrhea (painful menstruation) Other * headaches * organ pain * pain of injury * |
| ambigua | visceral pain |
| Cramp Bark - | Brain and Nervous System Conditions * all nervous conditions * convulsions * cramps and spasms * fits and lock-jaw * |
| Viburnum opulus | hysteria * nervous debility Cardiovascular Conditions * heart disease * palpitation Female Conditions Cramp Bark is one of |
| | the best female regulators in the herb world, being used for: * afterbirth cramping * internal hemorrhaging * menstrual |
| | cramps * preventing miscarriage * uterine sedative Inflammatory Conditions * rheumatism Respiratory Tract Conditions * |
| | asthma |
| Crataeva nurvala - | Genitourinary Conditions * atonic bladder (lack of normal tone) * benign prostatic hyperplasia (increase in volume of a tissue |
| Crataeva | or organ caused by the formation of and growth of new cells) [usually combined with Pygeum] * bladder tonic * chronic |
| nurvala | urinary tract infections * hypotonic bladder (reduced tone or tension) and incontinence and possibly enuresis (bedwetting) * |
| | prevention and treatment of kidney stones |
| Damiana - | Brain and Nervous System Conditions * anxiety and depression * brain tonic * nervous debility, combined with Saw Palmetto |
| Turnera diffusa, | * nervousness * nutrient for nerve tissue * tonic for the nervous system Cardiovascular Conditions * hypertension Female |
| Turnera | Conditions * regulates the female cycles Genitourinary Tract Conditions * urinary problems Reproductive Conditions |
| aphrodisiac | Damiana is reputed to be a male aphrodisiac, as it has an action on the male reproductive system similar to the male hormone, |
| | Testosterone, but is beneficial to both sexes. * benefits the reproductive organs * sexual debility, combined with Saw |
| | Palmetto * stimulates the sexual appetite * stimulates the sexual organs |
| Dan Shen - Salvia | Blood Conditions * blood stimulant * blood stasis remover with sedative action Brain and Nervous System Conditions * |
| militiorrhiza | increased cerebrovascular circulation * insomnia * nerve compression * diabetic neuropathy Cardiovascular Conditions |
| | Buerger's disease (blood vessel disease resulting in poor circulation in the lower legs, causing skin problems such as * burning |
| | * tingling * ulcerations * ischemic heart disease * angina * heart attacks * high blood pressure * increased peripheral |
| | circulation * palpitations * pulmonary fibrosis Female Conditions * menstrual problems Glandular Conditions * adjunct to |
| | treatment of lymphoma (a cancer that begins in the lymphatic system) Immune Conditions * autoimmune diseases * |
| | scleroderma Liver Conditions * acute and chronic liver disease Skin Conditions * acne * other skin disorders Other |
| | Conditions * impact injuries * promotes healing of fractures and other injuries |
| Dandelion - | Dandelion has two particularly important uses: * to promote the formation of bile * to remove excess water from the body in |
| Taraxacum | edemous conditions resulting from liver and/or gallbladder problems Dandelion root benefits all functions of the liver and |
| officinale | especially affects all forms of secretion and excretion from the body. By acting to remove poisons from the body, it acts as a |
| | tonic and stimulant and is helpful for: Blood Conditions * purifies the blood Brain and Nervous System Conditions * |
| | hypochondria * insomnia Cardiovascular Conditions * healthy function of the heart muscle * promotes healthy circulation |
| | Dandelion leaves can be used for: * fluid retention due to heart problems Female Conditions * premenstrual fluid retention |
| | Dandelion leaves can be used for: * premenstrual syndrome Gastrointestinal Conditions * dyspepsia with constipation * |
| | habitual constipation * improves appetite * improves digestion * stimulates sluggish digestion Genitourinary Conditions * |
| | reduces uric acid * gout * urinary infections Glandular Conditions * The Stems are used for * glandular swellings |
| | Inflammatory Conditions * arthritis * chronic rheumatism Liver Conditions * clears obstructions in the liver and kidneys |
| | (such as stones) * cholesterol distribution * detoxifies poisons that have accumulated in the liver, spleen, and gall bladder * |
| | gallstones * hepatic complaints in warm climates * liver problems * jaundice * reduces cholesterol Viral Conditions * inhibits |
| | the hepatitis virus Other * acne (good when combined with Stinging Nettle) * a slimming device * detoxifies poisons that |
| | have accumulated in the spleen * disorders of the spleen * fever * metabolic disturbances * stiff joints * brightens the eyes * |
| | helps clear skin eruptions * helps eczema * helps itchy, scaly rashes * clears yellow skin |
| Devil's Claw - | Inflammatory Conditions: * arthritis * fibrositis * muscular inflammation * rheumatism |
| Harpagophytum | |
| procumbens | |
| Dill - Anethum | Brain and Nervous System Conditions * insomnia (seeds) Gastrointestinal Conditions * colic in children * flatulence |
| graveolens | (gas/wind) * halitosis (bad breath) (seeds) * hiccoughs * indigestion * digestive aid * pain from flatulence (seeds) * stomach |
| | upset (seeds) Female Conditions * promotes milk in nursing mothers Other * children's ailments |
| Dong Quai - | Blood Conditions * aids in regulating blood sugar * anemia (increases red blood cell count) * blood purifier and general blood |
| Angelica sinensis | tonic for both men and women * blood stasis * decreases blood viscosity * helps prevent iron deficiency and anemia * |
| | improve bloods circulation * inhibits platelet aggregation (due fo Ferulic acid) * internal bleeding or bruising * promotes |
| | formation of red blood cells * reduces blood clotting * reduces thrombosis (works differently to Warfarin, possibly by |
| | inhibiting Thromboxane or decreasing fibrogen levels) * relaxes peripheral blood vessels Brain and Nervous System |
| | Conditions * insomnia * prevents spasms * relieves stress and calms the nerves * stimulates the central nervous system |
| | Cardiovascular Conditions * cardiac arryhthmia * cardiovascular disorders (with Dan Shen) * dilates blood vesselsheart |
| | disease (decreased symptoms of chest pain and improved exercise tolerance) when used in combination with Panax Ginseng |
| | and Astragalus * increases blood flow without affecting heart rate * lowers blood pressure * lowers vascular resistance * |
| | palpitations (with Dan Shen) * promotes blood circulation * reduces pulmonary hypertension * strengthens the heart * stroke |
| | (decreases the amount of brain damage following a stroke) Female Conditions (often combined with Paeonia) * amenorrhea * |
| | balances and treats many female systems and cycles * controls dysmenorrhea (painful menstruation) * endometriosis * female |
| | tonic * helps combat premenstrual tension * helps women resume normal menstruation after using birth control pills * |
| | improves fetal nourishment during pregnancy * infertility * infrequent periods * irregular menstruation (regulates the |
| | menstrual cycle) * menopausal symptoms, especially hot flashes * menstrual cramps * menorrhagia (excessive loss of blood |
| | during a period) * premenstrual syndrome (PMS), especially when combined with Black Cohosh * powerful herb for the |
| | female reproductive system * produces a balancing effect on estrogen activity * reduces spontaneous uterine contractions * |
| | regulates uterine contractions * regulates uterine function * stimulates as well as relaxes uterine muscles * stimulates the |
| | uterus during childbirth * strengthens internal reproductive organs * uterine bleeding * uterine cramping * weakness and |
| | headaches associated with menstrual disorders and for menopausal symptoms * hot flushes (especially in combination with |
| | other herbs) * vaginal dryness Gastrointestinal Conditions * alleviates constipation Genitourinary Conditions * strengthens |
| | the kidneys Immune System Conditions * hematological immunolgical problems (combined with Astragalus) Liver |
| | Conditions * chronic liver disorders (usually in combination with other herbs) * strengthens the liver Respiratory Tract |
| | Conditions * asthma * bronchitis Skeletal and/or Muscular Conditions * neuralgia * muscular-skeletal injuries Other |
| | Conditions * migraine headaches * reduces pain |
| Dragon's Blood - | Bacterial Conditions * syphilis Gastrointestinal Conditions * diarrhea * dysentery Respiratory Tract Conditions * asthma |
| Daemomorops | Externally: * gonorrhea (douche) * wound healing Other Uses: It is used as a coloring agent in: * imitation tortoiseshell * |
| draco | plasters * stains * tinctures * toothpaste * varnish |
| Echinacea - | Bacterial and Viral Conditions * AIDS * herpes * meningitis * vaginal yeast (candida) infections * venereal diseases Blood |
| Echinacea | Conditions * blood poisoning * cleansing the blood [one of the best blood purifiers] * hemorrhoids * increases the production |
| angustifolia, | of white blood cells Female Conditions * pelvic infections Gastrointestinal Conditions * dispels flatulence * fermentative |
| Echinacea | dyspepsia * improves digestion * promotes proper digestion Genitourinary Conditions * urinary tract infections Glandular |
| purpurea | Conditions * improves the lymph glands * relieves lymphatic swelling Immune System Conditions * boosts the body's |
| | immune system by increasing production of infection-fighting T-cells [T-lymphocytes] * helps build resistance to infections * |
| | helps in nearly all infections * stimulates a healthy immune system to ward off viruses * supports and stabilizes cellular |
| | immunity Combined with Golden Seal. Echinacea is effective against: * allergies * relieving the symptoms of hay fever (also |
| | called allergic rhinitis) * immune system disorders by increasing the process by which white blood cells destroy bad |
| | microorganisms * Candida (an annoying and persistent fungal infection) * staphylococcus and streptococcus infections |
| | Inflammatory Conditions * reduces inflammation * relieves arthritis Respiratory Tract Infections * bronchitis * sore throats * |
| | tonsillitis * helps build resistance to colds and influenza * reduces the severity of symptoms of influenza (flu) such as runny |
| | nose, fever and sore throat (pharyngitis) and reduces the duration of the illness * reduces the symptoms and duration of the |
| | common cold and alleviates the associated symptoms * sinusitis * tuberculosis * upper respiratory infections * whooping |
| | cough Other * aids in the production of interferon * cancers and tumors * ear infections (also known as otitits media) * fevers |
| | * relieves pain * skin cancers * ulcers * toothaches * chronic fatigue syndrome * indigestion * gastro-enteritis * weight loss * |
| | can stimulate wound healing and is beneficial internally and externally in skin conditions such as: * abscesses of teeth and |
| | glands * acne * athlete's foot * boils * burns * carbuncles * eczema * gangrene * insect bites * psoriasis * slow-healing |
| | wounds * ulcers |
| Elder Tree - | Blood Conditions * builds the blood * strengthens the walls damaged blood vessels Brain and Nervous System Conditions |
| Sambucus nigra | The berries are used for: * nerve disorders * neuralgia Elder Flowers are used for: * epilepsy Gastrointestinal Conditions The |
| | berries are used for: * laxative for small children * irritated and inflamed intestines Elder Leaves are used for: * constipation |
| | Genitourinary Conditions The berries are used for: * reduces inflammation of the urinary tract and bladder Elder Leaves are |
| | used for: * kidney problems * urinary problems * increasing the production of urine * eliminating excess water from the body |
| | Inflammatory Conditions The berries are used for: * arthritic and rheumatic complaints Elder Flowers are used for: * nasal or |
| | bronchial catarrh Respiratory Tract Conditions The berries are used for: * colds * influenza * relieves asthma and bronchitis * |
| | sore throat Elder Flowers are used for: * bronchitis * fighting influenza and colds as an excellent internal cleanser, mixed with |
| | Peppermint and Yarrow * quick recovery form the common cold and flu * sinusitis * soothing sore throats * soothing the |
| | respiratory system The flowers can be used for: * sore throats * tonsillitis Other The berries are used for: * back pain * |
| | induces perspiration to reverse the effects of a chill * lumbago * sciatica Elder Flowers are used for: * fevers * inducing sweat |
| | * obesity * cleansing the body * rheumatic complaints Externally the flowers have been used: * for conjunctivitis * for |
| | freckles * for rashes * for skin ailments, especially dry skin * for sunburn * to heal burns * to help relieve acne (with |
| | sassafras), eczema, psoriasis * to relieve headache (compress) Elder flowers, Elder flower water and Elder flower oil have |
| | been used in a variety of ways topically and as a tonic mixture. The leaves and flowers are used externally for: * burns * |
| | chapped skin * cuts * scalds * scrapes * swelling |
| Elecampane - | Blood Conditions * blood purifier * spitting of blood Brain and Nervous System Conditions * convulsions Cardiovascular |
| Inula helenium | Conditions * pulmonary conditions Female Conditions * menstrual problems Gastrointestinal Conditions * aids digestion * |
| | dyspepsia (with relaxation and debility) * intestinal worms * stimulates the digestive system * tones the stomach * chronic |
| | diarrhea Genitourinary Conditions * urinary tract infections * water retention Inflammatory Conditions * acute catarrh * |
| | respiratory and intestinal catarrh Respiratory Tract Conditions * asthma * bronchitis * chest infections * diseases of the lungs |
| | and breast * expels infected phlegm * quietens coughs * respiratory difficulties * respiratory tract infections * shortness of |
| | breath * whooping cough * chronic bronchitis * whooping cough Other * cramps * firms the gums * lessens tooth decay * |
| | protects against poisonous exhalations and bad air * strengthens eyesight Externally it is used for: * facial and other neuralgia |
| | * facial spots or blemishes * gout * hemorrhoids * itches * itching rashes * joint pains * putrid sores * scabies * sciatica * |
| | skin complaints * wounds Other Uses: * burned to repel insects * used in the preparation of Absinthe |
| Ephedra - | Blood Conditions * purifies the blood Genitourinary Conditions * flushes the kidneys Respiratory Tract Conditions * acts on |
| Ephedra vulgaris | air passages * asthma * hay fever * low blood pressure in influenza and/or pneumonia Inflammatory Conditions * relieves |
| | swellings of the mucous membranes * rheumatism * unstriped muscular fibers |
| Eucalyptus - | Gastrointestinal Conditions * dyspepsia * halitosis (bad breath) * indigestion Respiratory Tract Conditions * bronchitis * |
| Eucalyptus | common colds * coughs * microbial diseases of the lungs * pulmonary tuberculosis * sinusitis * sore throat Other Conditions |
| globulus | * diabetes * intermittent fevers * scarlet fever * typhoid Bacterial Conditions * diphtheria Cardiovascular System Conditions |
| | * increasing cardiac action Respiratory Tract Conditions * asthma * bronchitis * colds * coughs * influenza * loosens phlegm |
| | * sore throat Other Conditions * fainting * helps reduce fevers * helps reduce inflammation helps relieve chronic ear |
| | infections * burns * growths * infection * offensive smelling wounds * preventing sepsis * pyorrhea * sores * ulcers |
| Evening Primrose - | Brain and Nervous System Conditions * mental depression Cardiovascular Conditions * eases the pain of angina by opening |
| Oenothera | up the blood vessels * lowers blood pressure Female Conditions * female disorders associated with pelvic fullness * |
| biennis | menopausal symptoms * menstrual problems * relieves menstrual cramping (with Dong Quai and Chaste Berries) * |
| | symptoms of pre-menstrual syndrome (PMS) Gastrointestinal Conditions * dyspepsia * gastro-intestinal disorders of a |
| | functional origin * stimulates digestion Liver Conditions * hepatic torpor (sluggish liver) * lowers cholesterol levels * slows |
| | the production of cholesterol * stimulates the liver Respiratory Tract Conditions * spasmodic asthma * whooping cough Other |
| | * stimulates the spleen * to treat Multiple Sclerosis Externally it can be used for: * rashes * skin irritations |
| Eyebright - | Brain and Nervous System Conditions * strengthens a weak brain and memory Eye Conditions * conjunctivitis * weeping |
| Euphrasia | eyes * clears the sight * helps repair damage to eyes * strengthens the eyes in weak eyesight * acts specifically on the mucous |
| officinalis | linings of the eyes and all conditions aggravated by: * allergic reactions * airborne pollutants * car exhaust fumes * chemical |
| | sprays * cigarette smoke * smoky atmosphere Gastrointestinal Conditions * benefits the digestion * gastric disorders |
| | Inflammatory Conditions * catarrh * catarrhal and inflammatory problems of the nose and throat Respiratory Tract Conditions |
| | * colds in general * colds in the head * hay fever Other * headaches * mucous membrane conditions * chronic bronchitis |
| False Unicorn - | Gastrointestinal Conditions * dyspepsia * soothes a delicate stomach Genitourinary Conditions * genitourinary weakness or |
| Chamaelirium | irritability * kidney and liver disorders Female Conditions It is regularly used during menopause due to its positive effects on: |
| luteum | * depression * dragging sensation in the extreme lower abdomen * headaches * uterine disorders Liver Conditions * kidney |
| | and liver disorders * poor action of liver Male Conditions * impotence * spermatorrhea (involuntary release of semen) |
| | Reproductive System Conditions * stimulates the reproductive organs in women and men * weakness of the reproductive |
| | system |
| Fennel - | Blood Conditions * cleanses the blood Brain and Nervous System Conditions * sedative for small children Gastrointestinal |
| Foeniculum | Conditions * abdominal cramps * acidic stomachs * calms irritation from flatulence, griping pains and over-activity of the |
| vulgare | bowel * colic in infants and babies * digestive problems * digests fats efficiently * flatulence (gas/wind) * heartburn * |
| | hiccoughs * improves digestion * nausea * stomach and intestinal problems * stimulates appetite * stimulates sluggish |
| | digestion Female Conditions * increases milk supply in nursing mothers Genitourinary Conditions * gout * urinary stone |
| | formation (eases the pain and breaks down the stones) Liver * efficient digestion of fats * obstructions of the liver and gall |
| | Respiratory Tract Conditions * chronic coughs * eases coughs in children * expels mucous accumulations * shortness of |
| | breath * wheezing in the chest * coughing * hoarseness Other * cancer patients after radiation and chemotherapy treatment * |
| | obesity * obstructions of the spleen * spasms Externally it can be used for: * eyestrain * irritation of the eyes * soothing tired |
| | eyes * strengthening and restoring failing sight, combined with Rue * colic (rubbed on child's abdomen) * rheumatic pains |
| Fenugreek - | Blood Conditions * anemia Brain and Nervous System Conditions * neurasthenia Female Conditions * relaxes the uterus * |
| Trigonella | stimulates breast development * stimulates milk production in nursing mothers Gastrointestinal Conditions * comforts the |
| foenum-graecum | stomach * enteritis * faulty digestion (soothes the intestinal canal) * gastritis * increases the appetite * inflamed conditions of |
| | the stomach and intestines * softens and expels mucous * tonic effect on the digestion, particularly for convalescents Liver |
| | Conditions * counters cholesterol * used by the liver for cholesterol metabolism Metabolic Conditions * helps aid diabetes |
| | Respiratory Tract Conditions * bronchitis * dissolves mucous congestion * kills infections in the lungs * tuberculosis * throat |
| | and chest disorders Other * debility after illness * for weight gain * prevents fevers (similar to quinine) * reduces symptoms |
| | of fever * rickets * scrofula * soothes and nourishes the body during illness * sore throat Externally it is used for: * abscesses |
| | * boils * carbuncles * fistulas * gout pain * inflamed or infected skin * inflammations * neuralgia * sciatica * skin irritations |
| | * sores * swollen glands * tumors * drawing out ulcers and swellings * wounds |
| Feverfew - | Brain and Nervous System Conditions * delerium tremens (DT's) in alcoholics * dizziness * gives a sense of well-being * |
| Pyrethrum | hysterical complaints * low spirits. ‘down in the dumps’ * melancholy and weariness * migraine headaches [by controlling |
| Parthenium, | inflammation that constricts the blood vessels in the head] * nervousness and nervous conditions * promotes a restful sleep * |
| Chrysanthemum | reduces frequency and severity of headache Cardiovascular Conditions * lowers blood pressure Gastrointestinal Conditions * |
| parthenium, | digestive problems * colic * eases nausea and vomiting * expels worms * flatulence (gas/wind) * improves digestion * |
| Tanacetum | indigestion * stimulates appetite * stomach irritation Genitourinary Conditions * promotes adequate kidney function Female |
| parthenium | Conditions * expels afterbirth * induces menstruation * painful or sluggish menstruation * strengthens the womb |
| | Inflammatory Conditions * diseases caused by chronic inflammation, such as arthritis * more effective than other non- |
| | steroidal anti-inflammatories (NSAIDS). like aspirin * relieves inflammation and pain Respiratory Tract Conditions * asthma |
| | * colds * coughs * influenza * shortness of breath Feverfew * inhibits the release of serotonin and prostaglandins. |
| | inflammatory substances. which are believed to contribute * to the onset of migraines * produces the chemical Histamine |
| | Feverfew is often used as a treatment for disorders controlled by aspirin such as: * depression * fever * headaches (by |
| | preventing blood vessel spasms which may contribute to headaches) * inflammation and pain in arthritis sufferers * nausea |
| | Other * antidote in opium over use * dermatitis * has a tonic effect * relieves skin perspiration associated with migraines * |
| | tinnitus It has been used externally: * for itching of insect bites * for relieving the pain and swelling of insect bites (similar to |
| | Chamomile) * for wind and colic |
| Flax - Linum | Gastrointestinal Conditions * constipation * digestive disorders * encourages elimination by increasing the amount of fecal |
| usitatissimum | matter Genitourinary Conditions * gravel and stone * irritation of the urinary organs * urinary disorders Inflammatory |
| | Conditions * catarrh Liver Conditions * gallstones Respiratory Tract Conditions * colds * coughs * lung and chest problems * |
| | pleurisy Externally for: * abscesses * boils * bronchitis * deep-seated inflammation * drawing out swellings * removing spots |
| | or blemishes from the face, mixed with honey * rheumatic complaints * ulcers * burns * scalds |
| Frankincense - | Bacterial Conditions * gonorrhea * leprosy (China) Gastrointestinal Conditions * dysentery * fevers * ulcers * vomiting Other |
| Boswellia | * tumors Externally it has been used as an inhalant to relieve: * bronchitis * laryngitis * boils * carbuncles * gangrenous sores |
| thurifera | |
| Garlic - Allium | Bacterial and Viral Conditions * fights bacteria like an antibiotic * inhibits the growth of different species of bacteria * |
| sativa | stimulates the growth of healthy bacteria in the system * viruses Garlic is reported to be more effective than penicillin against: |
| | * the organisms responsible for cholera, dysentery and enteritis * paratyphoid disease * putrefactive intestinal bacteria * |
| | streptococcus and staphylococcus bacteria * typhus disease Blood Conditions * dissolves blood clots * reduces fat levels in |
| | the blood Brain and Nervous System Conditions * epilepsy * hysteria Cardiovascular Conditions * angina pectoris * |
| | arteriosclerosis * balances blood pressure * decreases triglycerides * helps maintain healthy circulation * helps prevents |
| | atherosclerosis (plaque build up in the arteries causing blockage and possibly leading to heart attack or stroke) * improves |
| | circulation * lowers blood pressure * may prevent blood clots * mild hypertension * prevents thrombosis (counteracts the |
| | tendency of clot forming cells to stick together within the blood vessels) * protects against cardiovascular disease * reduces |
| | blood pressure in hypertensive conditions * thins the blood (which reduces the risk of heart attack and stroke) Ear Conditions |
| | * earache * ear infection * otitis media, (an ear infection) Gastrointestinal Conditions * chronic stomach and intestinal catarrh |
| | * digestive infections * relieves belching and heaviness * relieves colic * relieves gas (flatulence) * relieves nausea * rids the |
| | body of intestinal parasites, especially pinworms * stimulates the activity of the digestive organs * ulcers Genitourinary |
| | Conditions * dropsy * urinary infections * fluid retention Immune System Conditions * fights infection * improves resistance |
| | to infection * increases the activity of white blood cells and T-helper cells (natural killer cells), the cells that are central to the |
| | activity of the entire immune system infections of the body * preventative measure for infectious diseases * stimulates the |
| | body's natural defenses against foreign invaders * protects cell membranes and DNA from damage Inflammatory Conditions |
| | * arthritis * infantile catarrh Liver Conditions * lowers cholesterol while increasing the level of beneficial HDL's (high- |
| | density lipoproteins) the so-called good cholesterol * may help lower homocysteine levels (similar to cholesterol which may |
| | contribute to increasing amounts of blood clots and plaque in blood vessels) * regularizes liver and gallbladder activity * |
| | stimulates the production of the liver's own detoxifying enzymes which neutralize carcinogens and other * environmental |
| | toxins Metabolic Conditions * balances blood sugar * late-onset diabetes Parasitic Conditions * Ascaris lumbricoides |
| | (roundworm) especially fresh, raw Garlic Respiratory Tract Conditions * asthma * breathing difficulties * chronic bronchitis |
| | * colds (reduces symptoms faster) * coughs and hoarseness * inhibits the growth of the bacteria, Mycobacterium tuberculosis, |
| | the organism responsible for tuberculosis (high doses) * preventative measure for colds and influenza * sinusitis * upper |
| | respiratory infections (especially infections deep in the lungs and throat and in the nasal passages or sinuses) Skin Conditions |
| | * acne * cutaneous eruptions * pimples Other Conditions less likely to have certain types of cancer especially: * cancer * |
| | colon cancer * stomach cancer * skin cancer offer some protection against the development of: * breast cancer * laryngeal |
| | (throat) cancer * prostate cancer * stimulates cell growth and activity * tumors * wounds Externally for: * abscesses * arthritis |
| | * dispelling hard swellings * earaches * insect bites * scrofulous sores * toothache * wounds |
| Gentian - | Blood Conditions * blood cleanser * raises the white blood cell count Brain and Nervous System Conditions * hysteria |
| Gentiana lutea | Gastrointestinal Conditions * dyspepsia * fainting spells * flatulence (gas/wind) * catarrhal gastritis with diarrhea * gastro- |
| | intestinal inflammations * griping pains and stitches * heartburn * improves appetite and poor digestion * indigestion * |
| | promotes the flow of digestive juices and bile * stomach ache * strengthens the activity of the stomach (take half an hour |
| | before meals) * vomiting * weakness of the digestive organs Female Conditions * female weaknesses Liver Conditions * |
| | biliousness * jaundice * promotes the flow of bile Other * anorexia * counters poisons, putrefaction and pestilence * general |
| | debility * discourages the smoking habit * intermittent fevers * relieves a fever Externally Gentian can be used: * for soothing |
| | inflammation * in footbaths * wounds |
| Ginger - Zingiber | Brain and Nervous System Conditions * prevents dizziness Cardiovascular Conditions * problems related to poor circulation |
| officinale | due to cold weather * promotes blood flow to the extremities * stimulates circulation * warming, anti-chilling effect on the |
| | whole circulation Female Conditions * during pregnancy for treating morning sickness * suppressed menstruation |
| | Gastrointestinal Conditions * alcoholic gastritis * diarrhea from a relaxed bowel where there is no inflammation * during |
| | pregnancy for treating morning sickness and digestive problems * dyspepsia * flatulent colic * helps with griping when added |
| | to other laxative herbs * stimulates digestion Respiratory Tract Conditions * at the onset of a cold to relieve usual symptoms * |
| | removes congestion * soothes sore throats * strengthens and heals the respiratory system Other * prevents vomiting * |
| | promotes cleansing of the system through perspiration * relieves headaches and body aches * relieves travel or motion |
| | sickness * spasms Externally Ginger can be used for: * chill blains * hand and footbath (dilute if there is broken skin) * sore |
| | and stiff neck |
| Ginkgo Biloba - | Blood Conditions * decreases capillary fragility * diminished blood flow to the head * hemorrhoids * inhibits platelet activity |
| Ginkgo biloba | factor (PAF), which is a common allergen in the body * varicose veins Brain and Nervous System Conditions Ginkgo Biloba |
| | is used for cerebral insufficiency which is a general term for a collection of symptoms including: * absentmindedness * |
| | anxiety * confusion * depressed moods * difficulties of concentration and memory * dizziness * headache * memory |
| | problems (currently being used in Alzheimer's Disease in its early stage and for other symptoms of cerebral insufficiency) and |
| | * boosts oxygen levels in the brain which uses 20% of the body's oxygen * elevates mood * enhances memory because of its |
| | positive effects on the vascular system, especially in the cerebellum * helps lack of energy * helps mental fatigue * improves |
| | mental alertness * improves short and long term memory * increases blood flow to the body's extremities including the head * |
| | increases blood flow to the brain and throughout the body's network of blood vessels that supply blood and oxygen to the |
| | organ systems * increases reaction time and improved mental clarity * neurological disorders * regulates neurotransmitters * |
| | relieves tension and anxiety * restores energy * tonic to the brain, central nervous system * vertigo Cardiovascular Conditions |
| | Ginkgo has been used to treat circulation-related disorders such as: * diabetic peripheral vascular disease * insufficient |
| | circulation and complications from skull injuries * insufficient circulation and complications from stroke * improving |
| | circulation in the extremities relieving cold hands and feet * opening of the smaller veins helping to improve circulation to all |
| | organs, especially the heart * Raynaud's syndrome * vascular constriction as well as * a tonic to the vascular system * chronic |
| | arterial blockage * circulation problems * helping control the transformation of cholesterol to plaque associated with the |
| | hardening of arteries * relaxing constricted blood vessels Genitourinary Conditions * improves kidney function Glandular |
| | Conditions * improves pancreas function Male Conditions * impotence Metabolic Conditions * increases metabolism |
| | efficiency Respiratory Tract Conditions * allergies that affect breathing * asthma * colds * relieves sinus congestion * |
| | stubborn coughs Other * a potential treatment to prevent the rejection of transplanted organs * countering the effects of aging |
| | * countering the effects of long term nicotine smoking * swelling in the limbs * tinnitus [ringing in the ears] when combined |
| | with Hawthorn and St Mary's Thistle * toxic shock syndrome * treats head injuries |
| Ginseng | Brain and Nervous System Conditions * influences neurological reactions * may slow the progression of Alzheimer's Disease |
| (American) - | * stimulates the central nervous system * boosts energy levels * depression * enhances and restores memory * enhances |
| Panax | concentration and cognitive abilities which may be impaired by improper blood supply to the brain * increases protein |
| quinquefolium | synthesis and activity of neurotransmitters in the brain * insomnia * reduces the effects of stress * removes both mental and |
| | bodily fatigue Cardiovascular Conditions * improves overall cardiovascular activity * decreases triglyceride levels * dissolves |
| | tumors * improves performance * may decrease endothelial cell dysfunction (which can cause blockage of blood flow leading |
| | to heart attack or stroke) * may raise HDL (the good cholesterol), while reducing total cholesterol levels * quiets down the |
| | blood vessels * relieves pulmonary complaints Female Conditions * improves sexual function * increases estrogen levels in |
| | women * menopausal symptoms Gastrointestinal Conditions * stimulates the appetite Glandular Conditions * benefits the |
| | endocrine system, especially the pituitary and adrenals * increases natural killer cell activity * prevents adrenal hypertrophy |
| | Immune System Conditions * aids in the production of thymic hormones, necessary for correct functioning of the immune |
| | system * stimulates the immune system Liver Conditions * reduces total cholesterol level * relieves nausea and vomiting |
| | Male Conditions * improves sexual function * may increase sperm count as well as motility * reduces impotence * stimulates |
| | the gonads Respiratory Tract Conditions * colds * chest problems Other Conditions * diabetes (appears to lower blood sugar |
| | [glucose] levels in Type 2 diabetes) * insulin-like activity * maintains excellent body functions * may reduce one's chances of |
| | getting various types of cancer, in particular: * lung * liver * stomach * pancreatic * ovarian * prevents excess corticosteroid |
| | production in response to physical, chemical or biological stress * radiation and chemotherapy protection * recovering from |
| | illness or surgery, especially for the elderly * reduces the effects of aging * stimulates the production of interferons Ginseng, |
| | whether it comes from Asia or America is for people who have: chronic fever excess craving excess hunger hot flushes thirst |
| Ginseng (Korean) - | Blood Conditions * blood diseases * hemorrhages Brain and Nervous System Conditions * alcoholism * balances and |
| Panax Ginseng, | counters the effects of stress * depression * drug abuse * enhances and restores memory * enhances concentration and |
| Panax schinseng | cognitive abilities (impaired by improper blood supply to the brain) * improves one's concentration * increases mental and |
| | physical efficiency * increases physical and mental activity [effects more pronounced in tired and weak people] * insomnia * |
| | may slow the progression of Alzheimer's Diseasemental disorders * mildly stimulates the nervous system * promotes mental |
| | and physical vigor * reduces the effects of stress * regulates the nervous system and exhibits normalizing effects on the body |
| | * relieves depression * stimulates and improves the work of the brain cells * stimulates the central nervous system |
| | Cardiovascular Conditions * aids in the prevention of heart disease * cardiac arrhythmia * cold limbs * cures pulmonary |
| | complaints * heart attacks * heart failure * increases blood circulation * long term debility and neurosis * lowers blood |
| | pressure * may decrease endothelial cell dysfunction (which can cause blockage of blood flow leading to heart attack or |
| | stroke) * may raise HDL (the good cholesterol), while reducing total cholesterol levels * normalizes blood pressure * |
| | palpitations * quiets down the blood vessels * regulates effects of high or low blood pressure * regulates the basal metabolic |
| | rate, often raising it when it is too slow, to increase the breakdown and metabolism of foods Female Conditions * eases |
| | childbirth * increases estrogen levels in women * normalizes menstruation During menopause Ginseng is helpful to: * balance |
| | hormones * improve moods (particularly feelings of depression) and give a sense of well-being * regulate hot flushes * |
| | rejuvenate the system Gastrointestinal Conditions * stimulates appetite Genitourinary System * increases the production of |
| | body fluids * removes more waste products Glandular Conditions * benefits the endocrine system, especially the pituitary and |
| | adrenal glands Immune System Conditions * improves resistance to infection * increases natural killer cell activity * increases |
| | resistance to stress and disease * stimulates the immune system Inflammatory Conditions * feverish and inflammatory |
| | illnesses * inflammatory conditions, combined with Licorice (due to Ginseng's effect on endogenous glucocorticoids) Male |
| | Conditions * enhances blood flow which makes it useful as an erection booster * improves sexual function and remedies |
| | impotence * poor sperm function (may increase sperm count as well as motility) * stimulates the gonads Respiratory Tract |
| | Conditions * asthma * colds * coughs * dyspnea (labored or difficult breathing) * improved respiratory function in severe |
| | chronic respiratory disease such as: * chronic bronchitis * emphysema * various chest problems Other Conditions * aids |
| | recovery from disease or surgery * alters the ability of the skeletal muscles to use free fatty acids rather than glucose for |
| | energy * boosts energy levels * may reduce one's chances of getting various types of cancer, in particular: * lung * liver * |
| | stomach * pancreatic * ovarian * debilitated and degenerative conditions, especially those associated with old age * diabetes |
| | (appears to lower blood sugar [glucose] levels in Type 2 diabetes) * dissolves tumors * enhances sports performance * help |
| | in sugar and fat metabolism * increases endurance * lowers cholesterol and decreases triglyceride levels * maintains excellent |
| | body functions * normalizes physical functioning depending on what the individual needs (e.g. it will lower high blood |
| | pressure but raise low blood pressure) * organ prolapse * overcomes the strain and drain of exercise with quicker recovery |
| | time, more efficient use of oxygen and less production of lactic acid * prostration * minimizes side effects of radiation and |
| | chemotherapy protection * recovering from illness or surgery, especially for the elderly * relieves fatigue * replenishes vital |
| | energy * spontaneous sweating Ginseng, whether it comes from Asia or America is for people who have: * chronic fever * |
| | excess craving * excess hunger * hot flushes * thirst |
| Ginseng | Brain and Nervous Conditions * The anti-stress factor of Siberian Ginseng increases human resistance to a wide variety of |
| (Siberian) - | stress factors. Siberian Ginseng: * counteracts threats to typical stress-induced illnesses * cases the strain of worry and bottled |
| Eleutherococcus | up anxiety * gives more mental alertness * helps nervousness * helps people cope better under the ordinary stress and tension |
| senticosus | of modern everyday life * helps the body adapt to stressful conditions * helps where there is excess corticosteroid production |
| | (from physical, chemical or biological stress) * improves sleep * increases stamina * memory problems * normalizes physical |
| | functioning depending on what the individual needs (e.g. it will lower high blood pressure but raise low blood pressure) * |
| | produces a protective effect against the stress of surgery, accidents, certain chemical toxins, radiation and chronic illnesses * |
| | produces a soothing and calming effect on people who have endured months of pressure and tension * relieves depression * |
| | relieves tensions of business and sports competitions * restores vigor * stimulates a good memory * supports the central |
| | nervous system Siberian Ginseng has a pronounced effect on functional nervous disorders, such as patients suffering from: * |
| | nervous and emotional disturbances (not insanity) * nervous exhaustion Cardiovascular Conditions * lowers blood pressure * |
| | normalizes low blood pressure and mild forms of high blood pressure * supports the circulatory system Female Conditions |
| | During menopause Ginseng: * aids in rejuvenating the system * balances hormones * regulates hot flushes Gastrointestinal |
| | Conditions * digestive troubles * stimulates a healthy appetite Immune System Conditions * stimulates the immune system * |
| | helps the body deal with physically and mentally stressful exposures such as: * bacteria * chemicals * cold * extreme working |
| | conditions * heat * noise * physical exhaustion * pollution * viruses Liver Conditions * supports liver function Muscular |
| | Conditions * increases muscle strength Reproductive System Conditions * increases reproductive capacity * male infertility * |
| | stimulates sexual organs Respiratory Tract Conditions * supports lung function Viral Conditions * helps the body resist viral |
| | infections and environmental toxins * slows the replication of certain viruses including: * influenza A (which causes the flu) * |
| | human rhinovirus (causes symptoms of the common cold) * respiratory syncytial virus (causes symptoms of the common |
| | cold) * reduced frequency, severity, and duration of outbreaks of herpes simplex virus type 2 (which generally causes oral |
| | herpes lesions) Other Conditions * cancers * enhances overall health * enhances productivity * increases energy, stamina and |
| | endurance levels * increases longevity * overcomes all forms of illness, physical and mental * overcomes fatigue * recovering |
| | from illness or surgery, especially for the elderly * reduces elevated sugar content in mild and moderate cases of diabetes * |
| | restores loss of vitality * tones the body during stress Ginseng, whether it comes from Siberia, Asia or America is for people |
| | who have: * chronic fever * excess craving * excess hunger * hot flushes * thirst |
| Ginseng | Blood Conditions * hematemesis (vomiting of blood) * hematoma (tumor like mass produced by coagulation of extravasated |
| (Tienchi) - Panax | (escape of fluid into the tissues) blood in a tissue or cavity * hematuria (blood in the urine) * hemoptysis (coughing and |
| notoginseng, | spitting of blood as a result of bleeding from the respiratory tract * hemorrhage and pain caused by trauma * metrorrhagia |
| Panax | (uterine bleeding at irregular intervals) * moves blood through the body * stops bleeding * transforms congealed blood * |
| pseudoginseng | traumatic injury with bruising Cardiovascular Conditions * angina pectoris * arrhythmia * myocardial ischemia * optimizes |
| | circulation Immune Conditions * enhances defense system balance Other Conditions * alleviates and reduces pain * chest and |
| | abdominal pain * high blood cholesterol * may slow the progression of Alzheimer's Disease * normalizes physical |
| | functioning depending on what the individual needs (e.g. it will lower high blood pressure but raise low blood pressure) * |
| | recovering from illness or surgery, especially for the elderly * reduces swelling * useful for weight loss |
| Goat's Rue - | Gastrointestinal Conditions * worms Female Conditions * increasing milk supply in nursing mothers Metabolic Conditions * |
| Galega officinalis | diabetes * lowers blood sugar levels (seeds) Other * fevers Externally a footbath containing Goat's Rue is good for: tired, |
| | aching feet, especially from over walking |
| Goldenrod - | Bacterial Conditions * diphtheria Blood Conditions * bleeding from the intestines * internal hemorrhage Gastrointestinal |
| Solidago | Conditions * diarrhea * disorders of the intestines Genitourinary Conditions * chronic nephritis * kidney and bladder stones * |
| virgaurea | kidney problems, when indicated by dark, cloudy urine * urinary tract infections combined with Hawthorn, Agrimony and |
| | Thyme Female Conditions * menorrhagea (excessive menstruation) Inflammatory Conditions * arthritis * catarrhal problems |
| | Respiratory Tract Conditions * laryngitis and pharyngitis * long term, low-grade nasal catarrh where the infection is stubborn |
| | * upper respiratory catarrh * whooping cough Other * eczema It is used externally for: * bruises * chronic eczema * insect |
| | bites * ruptures * scar tissue * sores * ulcers * wounds |
| Goldenseal - | Bacterial and Viral Conditions * antibacterial remedy for the mucous membranes of the body * destroys many types of |
| Hydrastis | bacterial and viral infections Blood Conditions * ability to tighten blood vessels * hemorrhages Cardiovascular Conditions * |
| canandensis | promotes the functioning capacity of the heart Female Conditions * childbirth (induces contractions) * controls heavy |
| | menstrual and postpartum (after birth) bleeding * menstrual disorders * uterine conditions such as menorrhagea (excessive |
| | menstruation) Gastrointestinal Conditions * all stomach ailments * astringent on the mucous membranes that line the gut * |
| | chronic inflammation of colon or rectum * disordered and debilitated states of the digestive system * habitual constipation * |
| | healthy functioning of the colon * increases digestive secretions * colitis * dyspepsia * gastric catarrh * gastritis * healthy |
| | functioning of the colon * loss of appetite * peptic ulcers * promotes the production of saliva, bile, and other digestive |
| | enzymes * reduces gastric inflammation * tonic to mucous membranes of digestive system * various gastric disorders |
| | Genitourinary Conditions * stimulates the kidney function * urinary infections * various genitourinary disorders Glandular |
| | Conditions * healthy functioning of the pancreas * promotes the functioning capacity of the lymphatic system * healthy |
| | functioning of the spleen, pancreas, and colon Immune System Conditions * strengthens the immune system Inflammatory |
| | Conditions * inflamed catarrhal conditions of the mucous membranes * rheumatic and muscular pain Liver Conditions * |
| | biliary tract conditions * healthy functioning of the liver * liver troubles Metabolic Conditions * potentizes insulin |
| | Respiratory Tract Conditions * infection, inflammation or congestion of lungs, throat and sinuses * promotes the functioning |
| | capacity of the respiratory system * symptoms of the cold and flu for: * gum disease * inflamed gums * pyorrhea * sore |
| | throats * throat infections * tonsillitis Other * chronic alcoholism, combined with Cayenne * cleanses vital organs * |
| | disordered and debilitated states of the mucous membranes * healthy functioning of the spleen * night sweats * relieves |
| | congestion * tonic to all mucous membranes * vomiting Externally: * leuccorrhea * yeast infections |
| Gotu Kola - | Blood Conditions * aids in blood diseases * hemorrhoids * improves the flow of blood throughout the body by strengthening |
| Hydrocotyle | the veins and capillaries * phlebitis (inflammation of the veins) * purifies the blood * varicose aveins * heals blood vessels |
| asiatica, Centella | Brain and Nervous System Conditions * aids in restfull sleep * brain cell stimulant * brain energizer and preserver * brain |
| asiatica | nutritive * calming properties * excellent mental stimulant * fatigue, both mental and physical * for children with Attention |
| | Deficit Disorder (ADD) because of its stimulating effect on the brain (increasing one's ability to focus while having a soothing |
| | and relaxing effect on an overactive nervous system) * keeps one calm for yoga and meditative practices * improves and |
| | enhances memory * improves mental functions such as concentration * insomnia * mental disorders * prevents nervous |
| | breakdowns * rebuilds energy reserves * recovery after mental breakdowns * relieves mental fatigue * senility * stimulates |
| | the central nervous system * supports the central nervous system Cardiovascular Conditions * high blood pressure * |
| | congestive heart failure * venous insufficiency Gastrointestinal Conditions * bowel complaints Glandular Conditions * gland |
| | tonic Immune System Conditions * aids the body in defending itself against toxins * localized inflammation and infection * |
| | for people who are inactive or confined to bed due to illness Inflammatory Conditions * arthritis * rheumatism * skin |
| | inflammations Liver Conditions * hepatitis Respiratory Tract Conditions * sore throat * tuberculosis Tissue Development * |
| | connective tissues * keratinization (the process of replacing skin after sores or ulcers) * lymph tissue * promotes healthy skin |
| | * stimulation of hair and nail growth * synthesis of collagen (the first step in tissue repair) * supports the repair of cartilage * |
| | for mucous membranes * for skin * stimulates the formation of lipids and proteins necessary for healthy skin Other * |
| | balancing tonic that both increases energy and relaxes the body * destroys tumor cells * fever * ‘heaviness’ or tingling in the |
| | legs * leg cramps * post-surgery recovery * swelling of the legs Gotu Kola is used in Ayurvedic Medicine and is * regarded |
| | as one of the most spiritual and rejuvenating herbs in Ayurveda * said to develop the crown chakra, the energy center at the |
| | top of the head * used to balance the right and left hemispheres of the brain, which the leaf is said to resemble * used to |
| | fortify the immune system, cleansing and feeding it * used to improve meditation * used for revitalizing the nerves and brain |
| | cells * used to strengthen the adrenals Externally Gotu Kola has been used to treat: * cancer * leprosy * second and third |
| | degree burns * skin disorders * wounds It has been shown to: * reduce scar tissue formation Externally it is used for: * |
| | syphilitic ulcers |
| Gravel Root - | Brain and Nervous System Conditions * neuralgia Genitourinary Conditions * chronic renal and cystic problem * difficult and |
| Eupatorium | painful urination * dropsy * gout * gravel * hematuria (blood in the urine) * kidney problems Gastrointestinal Conditions * |
| purpureum | dyspepsia * indigestion, especially in elderly people Inflammatory Conditions * catarrh * rheumatism Respiratory Conditions |
| | * influenza Other * debility in general * intermittent fever * tapeworm |
| Green Tea - | Genitourinary Conditions promote the excretion of urine, astringent (to control bleeding and help heal wounds), and to |
| Camellia sinensis | improve heart health. Other traditional uses of green tea include treating flatulence, regulating body temperature and blood |
| | sugar, promoting digestion, and improving mental processes. |
| Guaiacum Resin - | Bacterial Conditions * secondary syphilis Blood Conditions * blood putrefaction, combined with Sarsaparilla * purifies blood |
| Guaiacum | Genitourinary Conditions * gout (relieves pain and inflammations between attacks and can reduce recurrence) Respiratory |
| officinale | Tract Conditions * tonsillitis Other * chronic rheumatism * rheumatoid arthritis * scrofula |
| Gymnema - | Gastrointestinal Conditions * reduces appetite * reduces sweet cravings Liver Conditions * Diabetes Other * develops a |
| Gymnema | higher ratio of muscle mass to body fat |
| sylvestre | |
| Hawthorn - | Hawthorn is a good preventative herb for people with a family history of * angina pectoris * arteriosclerosis * hardening of |
| Crataegus | the arteries * heart attacks * high or low blood pressure * valvular insufficiency * inflammation of the heart muscle * |
| oxyacantha | irregular pulse Hawthorn is used for: Blood Conditions * inflammation of the blood vessels * strengthens the walls of blood |
| (leaves), | vessels * varicose veins Brain and Nervous System Conditions * enhances poor memory by improving circulation of blood |
| Crataegus | within the head and increasing the amount of oxygen to the brain, when combined with Ginkgo Biloba * increases blood flow |
| monogyna | to the brain Cardiovascular Conditions * angina, a disease marked by intense chest pain * arteriosclerosis * cardiac curative * |
| (berries) | enhances the strength of the heart's contractions * heart failure and debility * heart muscle weakened by age * helps prevent |
| | irregular heartbeats called arrhythmias, which can lead to heart attacks * helps protect the heart against oxygen deprivation by |
| | inhibiting free radical formation which is beneficial in maintaining healthy heart vessels and promoting overall heart health * |
| | improves blood supply to the heart * improves circulation and increases tolerance for physical exertion * increases blood flow |
| | to the heart and brain * increases metabolism in the heart muscle * lowers blood pressure (with extended use) * lowers |
| | cholesterol and the amount of plaque in arteries * myocarditis (inflammation of the heart muscle) * nervous heart problems * |
| | normalizes blood pressure by regulating the action of the heart, not only lowering high blood pressure but also raising blood |
| | pressure that is low * normalizes cardiovascular functions * normalizes heart action * palpitations * rapid heart beat * reduces |
| | blood pressure and stress to the heart muscle * relaxes and dilates the arteries * restorative after a heart attack * stabilizes and |
| | strengthens the heartbeat * strengthens a heart muscle weakened by age * supports the heart * weak heart, combined with |
| | Rosemary and Rue Hawthorn Berries are used for: * congestive heart failure and circulatory disorders * increasing coronary |
| | blood flow * mild cardiac insufficiency Gastrointestinal Conditions * digestive problems, combined with Cactus grandiflorus |
| | Genitourinary Conditions * helps rid the body of excess salt and water thus supporting weight-loss and weight control |
| | programs * urinary tract infections. combined with Agrimony. Thyme and Golden Rod Respiratory Tract Conditions * sore |
| | throat |
| Hemidesmus | Bacterial Conditions * depraved conditions of the system * gonorrheal neuralgia * inveterate syphilis * pseudo-syphilis * |
| indicus | mescurio-syphilis * struma in all its forms * thrush * venereal disease Genito-Urinary Conditions * nephritic complaints |
| | Immune System Conditions * autoimmune disease such as * rheumatism * rheumatoid arthritis * Although mildly |
| | immunosuppressant Hemidesmus is very safe and non-toxic Lymphatic Conditions * scrofula Skin Conditions * chronic skin |
| | diseases * chronic ulcers Other Conditions * sore mouths of children |
| Hops - Humulus | Blood Conditions * purifies the blood Brain and Nervous System Conditions * calming effect on the nervous system * |
| lupulus | delirium tremens (in alcoholics or drug withdrawals) * fits * hysteria * induces sleep without causing headache * insomnia * |
| | nervous diarrhea * nervous spasmodic conditions * nervous tension * neuralgia * reduces a person's sensitivity to pain * |
| | relaxes the muscular activity of the nervous system * restlessness Cardiovascular Conditions * angina pectoris especially |
| | combined with Yarrow, Valerian and Lemon Balm * heart disease Gastrointestinal Conditions * digestion * improving the |
| | appetite * indigestion * intestinal cramps * irritable bowel syndrome * lack of appetite * mucous colitis * relaxes the |
| | muscular activity of the digestive system Genitourinary Conditions * bladder ailments * calculus problems * excess uric acid |
| | production * water retention Liver Conditions * jaundice * liver ailments * sluggish liver Male Conditions * curbs excessive |
| | sexual desire for males * spermatorrhea (excessive manufacture of sperm) Respiratory Tract Conditions * coughs Other * |
| | reduces a person's susceptibility to fevers * reduces fevers and pain * removes poisons from the body * soothes inflammation |
| Horehound, | Brain and Nervous System Conditions * nervous heart conditions Cardiovascular Conditions * calms heart action |
| White - | Gastrointestinal Conditions * tonic for the stomach * helps the digestion and liver function Liver Conditions * helps the liver |
| Marrubium | function * stimulates the flow and secretion of bile from the gallbladder Respiratory Tract Conditions * acute and chronic |
| vulgare | bronchitis * all lung conditions * asthma * chest, nasal and sinus congestion * children's cough remedies * coughing spells * |
| | coughs and chest complaints * dry unproductive coughs, with associated poor appetite and debility of the digestive system * |
| | eases tightness of the chest * encourages the production and expulsion of phlegm from the lungs * prickly sensation in throat |
| | and nasal area just before a cold sets in * smoker's cough, especially combined with Coltsfoot, Hyssop and Marshmallow |
| | Root tonic for the respiratory system and stomach Other * expels worms * restores the normal balance of secretions by |
| | various organs and glands of the body * typhoid and paratyphoid fever It is sometimes combined with Hyssop, Rue, Licorice |
| | Root and Marshmallow Root for greater efficacy Externally it can be used for: * temporary or persistent skin problems |
| Horseradish - | Blood Conditions * purifies the bloodstream Gastrointestinal Conditions * colitis * intestinal problems due to putrefaction * |
| Armoracia | stimulates digestion * worms in children Genitourinary Conditions * bladder infections * calculus (stones) problems * dropsy |
| lapathifolia, | * gouty pains Nervous System Conditions * stimulates the nervous system Respiratory Tract Conditions * asthma * catarrh of |
| Cochlearia | the lungs * clears up a persistent cough * clears up dripping sinuses * loosens chest congestion * hoarse voice * sinus and |
| armoracia | antrum congestion * whooping cough Other * promotes perspiration * rheumatic pains * rids the body of mucous wastes * |
| | warms a cold body Externally: * for aching joints * for chronic rheumatism * for facial neuralgia * for chill blains * for gout * |
| | for hard swellings of the liver and spleen * for sciatica * in paralytic afflictions * to massage away muscular aches * to |
| | stimulate blood flow |
| Horsetail - | Blood Conditions * anemia * bleeding of the lungs, uterus and stomach * blood staunching (to stop bleeding) * decreases |
| Equisetum | bleeding * helps in coagulation * hemorrhages (internal and external) * hemorrhoids * spitting of blood * vomiting of blood |
| arvense | Brain and Nervous System Conditions * depression, delusion and fits of rage, with Yarrow and Nettle * nerves |
| | Cardiovascular Conditions * prevents fatty deposits in the arteries Female Conditions * excessive menstrual flow * leucorrhea |
| | Gastrointestinal Conditions * cleanses the stomach * polyps in the abdomen or anus * stomach ulcers * stomatitis |
| | Genitourinary Conditions * bedwetting in children * bladder catarrh * cystitis * difficulty in urinating * dropsy * gout * |
| | incontinence * kidney and bladder trouble including gravel and stones * painful urination * promotes the expulsion of fluid |
| | through the urinary system * prostate problems * tones and heals further damage to the urinary system * urinary tract |
| | infections * water retention associated with kidney disorders Inflammatory Conditions * rheumatism * rheumatoid arthritis |
| | Respiratory Tract Conditions * accumulation of water in the pericardium or pleura * chronic bronchitis * fistulas and |
| | adenoids in palate or throat * inflammation or bleeding of the gums * inflammation of the mucous membranes of the mouth * |
| | tonsillitis * tuberculosis of the lungs Other * cramp-like pains * encourages the absorption and use of calcium by the body * |
| | general debility * helps brittle nails and hair (due to its high silica content) * helps broken bones heal faster * inhibits tumors |
| | * vomiting * white spots on nails Externally Horsetail is used for: * barber's itch * bony projections on the heel * broken nails |
| | * caries * cancer-like growths * cracked feet * external bleeding * fistulas * hemorrhoids * herpes * itching rashes, even if |
| | they are festering or scabby * inflammation of the nail bed * old festering wounds * nose bleeding * bursitis * drawing out |
| | foul smelling pus from wounds and sores * healing of wounds * liver and gallbladder attacks * painful congestions which |
| | press upon the heart * skin eruptions * stomach pains * ulcers * disc lesions not caused by a pinched nerve * leucorrhoea |
| | (white discharge in women) * lifeless hair * relieves kidney and bladder problems, especially stones in the bladder * the |
| | treatment of athlete's foot * tired feet |
| Hyssop - | Brain and Nervous System Conditions * eases nervous tension and anxiety * hysteria * petit mal (mild epilepsy) * soothing |
| Hyssopus | influence on the nervous system Gastrointestinal Conditions * chronic catarrh of the digestive system * flatulence (gas/wind) |
| officinalis | * poor digestion * improves the tone of a weak stomach * mucous congestion in the intestines Female Conditions * breast |
| | problems Inflammatory Conditions * relieves inflammations * rheumatism Liver Conditions * jaundice Respiratory Tract |
| | Conditions * asthma * bronchitis * chronic catarrh of the respiratory system * coughs due to colds * head colds * influenza * |
| | lung ailments * nose and throat infections * pulmonary complaints * sinusitis * soothes sore throats * chronic catarrh * sore |
| | throat, with Sage Viral Conditions * inhibits the growth of the herpes simplex virus Other * chronic catarrh (often combined |
| | with Horehound) * promotes perspiration at the start of fevers * scrofula * to kill internal worms Externally Hyssop has been |
| | used: * for bruises and discolored contusions * for burns * for cuts * for skin irritations * for swellings * for the relief of |
| | muscular rheumatism * to aid in healing of wounds * to bathe tired and aching eyes * head itch * head lice |
| Indian Corn - Zea | Bacterial Conditions * gonorrhea Cardiovascular Conditions * fluid in the heart Genitourinary System Conditions * acidosis * |
| Mays | bedwetting in children or older people, combined with Agrimony * bladder affections * bladder irritation from uric acid and |
| | phosphatic gravel, combined with Willow Herb * cystitis (chronic or acute) * formation of stones * gout * incontinence * |
| | kidney stones * nephritis * oliguria * neutralizes and promotes excretion of waste products containing uric acid * purulent |
| | decompositions or urine in the bladder * pyelitis * renal colic Inflammatory Conditions * rheumatism Other * edemous |
| | conditions * obesity |
| Inula Racemosa | Cardiovascular Conditions * angina pectoris * heart disease * ischemic heart disease, especially combined with Commiphora |
| | mukul * lowers diastolic blood pressure Liver Conditions * decreases cholesterol Respiratory Tract Conditions * asthma * |
| | bronchial disease * chest (precordial) pain * chronic bronchitis with cardiac complications * cough * dyspnea * protects |
| | against bronchospasm induced by histamine, serotonin and pollens |
| Juniper - | Cardiovascular Conditions * angina pectoris * heart disease * ischemic heart disease, especially combined with Commiphora |
| Juniperus | mukul * lowers diastolic blood pressure Liver Conditions * decreases cholesterol Respiratory Tract Conditions * asthma * |
| communis | bronchial disease * chest (precordial) pain * chronic bronchitis with cardiac complications * cough * dyspnea * protects |
| | against bronchospasm induced by histamine, serotonin and pollens |
| Kava Kava - | Bacterial Conditions * gonorrhea Cardiovascular Conditions * fluid in the heart Genitourinary System Conditions * acidosis * |
| Piper | bedwetting in children or older people, combined with Agrimony * bladder affections * bladder irritation from uric acid and |
| methysticum | phosphatic gravel, combined with Willow Herb * cystitis (chronic or acute) * formation of stones * gout * incontinence * |
| | kidney stones * nephritis * oliguria * neutralizes and promotes excretion of waste products containing uric acid * purulent |
| | decompositions or urine in the bladder * pyelitis * renal colic Inflammatory Conditions * rheumatism Other * edemous |
| | conditions * obesity |
| Lavender, French - | Bacterial and Viral Conditions * retroviruses, such as herpes * streptococcal and staphylococcus infections Brain and Nervous |
| Lavandula | System Conditions * fainting * gentle, soothing tonic for the nervous system * invigorates a tired brain * mental depression * |
| officinalis | migraine headache * nervous anxiety * nervous tension * stress * tension headaches Gastrointestinal Conditions * colic * |
| | flatulence (gas/wind) * gentle, soothing tonic for the digestive system * nausea * putrefactive bacteria in the intestines * |
| | stomach problems * vomiting Female Conditions * expulsion of afterbirth * induces menstruation Respiratory Tract |
| | Conditions * coughs * debility and exhaustion * dizziness Externally is used for: * acne * reducing puffiness * normalizing |
| | the sebaceous glands * repelling insects * leucorrhea * burns * corneal ulcers * cuts * disinfecting wounds * headache relief * |
| | toothache * intestinal gas * neuralgic pains * rheumatism * sore joints * sprains and * stimulates paralyzed limbs * relieves |
| | fatigue |
| Lemon Balm - | Lemon Balm is used to treat children with: colds colic fever influenza In adults Lemon Balm is used for: Brain and Nervous |
| Melissa | System Conditions * Alzheimer's disease * attention deficit/hyperactivity disorder (ADHD) * calms nervous people * |
| officinalis | depression * helps reduce anxiety * helps tired brains * hysteria * insomnia * lifts the spirits * melancholia * migraine |
| | headaches * nervous tension * sharpens memory and understanding * stress related conditions Cardiovascular System |
| | Conditions * tones the circulatory system Female Conditions * cleanses the womb after a miscarriage or abortion, especially |
| | if combined with Basil * expels afterbirth * flatulence (gas/wind) * headaches and dizziness during pregnancy * induces |
| | menstruation * menstrual cramps Gastrointestinal Conditions * abdominal crampscolic * eases pain and discomfort associated |
| | with digestion * flatulence and bloating * improves appetite * indigestion * maintains the health of the digestive system * |
| | nausea and vomiting * nervous dyspepsia * nervous stomachs * relieves spasms of the digestive tract Genitourinary |
| | Conditions * gout Glandular Conditions * hyperthyroidism (Grave's disease) Inflammatory Conditions * catarrh Muscular |
| | Conditions * calms muscle spasms Respiratory Tract Conditions * certain forms of asthma * chronic bronchial catarrh * |
| | clears the head * colds * influenza Viral and Bacterial Conditions * cold sores * fights infectious bacteria * genital herpes * |
| | herpes simplex * HIV * kills bacteria Other Conditions * fevers and feverish conditions * headaches * hyperthyroidism * |
| | induces perspiration * numbs tissues * relieves pain caused by external force * aching teeth * sore, inflamed throats |
| | Externally to aid in healing the crushed leaves are applied to: * boils * insect bites * sores * stings * tumors * wounds * |
| | depression |
| Licorice - | Cardiovascular Conditions * heart problems Female Conditions * symptoms of menopause * regulates menstruation * relieves |
| Glycyrrhiza | menstrual cramps Gastrointestinal Conditions * beneficial to the digestive system * catarrhal conditions of the intestinal tract |
| glabra | * eases spasms in the intestines * flatulence (gas/wind) * gastric irritation other gastric disturbances * gastric and peptic ulcers |
| | (forms a coating of thick protective mucilage over the ulcer site) * good laxative for children * heartburn * indigestion * |
| | loosens the bowels in children * lowers stomach acid levels (coats the stomach wall with a protective gel) * mild laxative |
| | effect on the lower digestive tract * stomach and intestinal ulcers * stomach problems Genitourinary System Conditions * |
| | Addison's disease * bladder and kidney ailments * catarrhal conditions of the urinary tract * dropsy * treatment of the kidneys |
| | Glandular System Conditions * adrenal gland problems * normalizes and regulates hormone production stimulates the |
| | production of hormones, such as the adrenal cortex hormones aldosterone and hydrocortisone which have anti-inflammatory |
| | properties * regulates low blood sugar * stimulates adrenal glands Inflammatory Conditions * arthritis * relieves arthritic and |
| | allergy symptoms (acts like cortisone but without the side effects) * relieves rheumatism * soothes irritated mucous |
| | membranes Liver Conditions * hepatitis * treatment of the liver * lowers blood cholesterol levels * stimulates the flow of bile |
| | Respiratory Tract Conditions * bronchitis and bronchial distress (increases the secretion of the bronchial glands) * chest |
| | infections and chest complaints in general * coughs (as effective as codeine, but safer, when used as a cough suppressant) * |
| | hoarseness * laryngitis * lung congestion * mucous congestion * sore throat * strengthens a singer's throat * throat irritations |
| | * tickling sensation in throat * wheezing Other * general tonic * neutralizes certain toxins * reduces fevers * removes thirst * |
| | treatment of the spleen * when recovering from an illness (supplies recuperative energy to the system) Externally used for * |
| | dermatitis * opening skin pores * skin infections |
| Marshmallow - | Gastrointestinal Conditions * beneficial for the digestive system * beneficial where the natural mucous has been abraded from |
| Althaea | the coats of the intestines * calms enteritis and colitis * colitis * digestive problems * dysentery * inflammatory bowel |
| officinalis | diseases such as: * Crohn's disease * ulcerative colitis * soothes stomach inflammation and ulcers Genitourinary System |
| | Conditions * cystitis * eases urinary pain * gravel * relaxing effect on the urinary passages * urinary hemorrhages * soothes |
| | infection and irritation from kidney and bladder stones Inflammatory Conditions * beneficial for joints (anti-irritant and anti- |
| | inflammatory) * soothes irritated tissue Respiratory System Conditions * aids in the expectoration of difficult mucous and |
| | phlegm from the lungs * all lung ailments including bronchitis * asthma * bronchial asthma * bronchitis * common cold * |
| | eases dry, harsh coughs * hoarseness (‘frog in the throat’) * lung catarrh * protects and soothes irritated mucous membranes * |
| | relaxes and soothes the bronchial tubes * removes mucous obstructions from the chest and throat (with Mullein) * soothes |
| | emphysema * soothes sore throats * whooping cough Other Conditions * aids weight loss (Marshmallow swells with fluid and |
| | gives a sense of fullness) Externally: It is used for * an eye for dry tear ducts * a vaginal * blood poisoning (often combined |
| | with Cayenne) * bruises * burns * chapped skin * earache * gangrene * minor wounds * muscle and sinew aches * pain, |
| | inflammation and swelling caused by insect bites and stings * rectal irritation * removing obstinate inflammations, often |
| | combined with Slippery Elm * skin inflammation * sprains |
| Meadowsweet - | Bacterial/Viral Conditions * eruptive infections such as * chickenpox * measles Blood Conditions * affections of the blood |
| Spiraea ulmaria | Brain and Nervous System Conditions * release spasm and induce restful sleep * relieves headaches * relieves neuralgia |
| | Female Conditions * menstrual cramps Gastrointestinal Conditions Meadowsweet is one of the best antacid remedies for acid |
| | conditions. * acid indigestion * colic * diarrhea in children * dyspepsia * excess acidity * gastric ulceration * gastritis * |
| | headaches * heartburn * helps to heal and to reduce acidity * hiatus hernia * indigestion * inflammatory condition of the |
| | stomach or bowels * irritable bowel syndrome * peptic ulcers * protects and heals the mucous membranes * rebuilds the |
| | digestive system during recovery from drug and alcohol abuse * relieves enteritis * relieves wind and distension * stomach |
| | and duodenal ulcers * stomach problems Genitourinary System Conditions * atherosclerosis in the arteries * bladder and |
| | kidney ailments * cystitis * dropsy * fluid retention * gout * gravel * helps eliminate toxic wastes and uric acid from the |
| | system * incomplete urination * kidney problems * kidney stones * urethritis * water retention Inflammatory Conditions * |
| | arthritis * combats infection * joint problems (which are associated with acidity) * rheumatism * swollen joints Muscular |
| | Conditions * aches and pains * muscle and joint pain Respiratory System Conditions * influenza * respiratory tract problems |
| | Skin Conditions * clears the skin * resolves rashes Other * antidote to infections and poisons * reduces fever * helps to soothe |
| | pain * promotes sweat * protects and soothes mucous membranes Externally it is used for: * sore eyes * wounds |
| Mistletoe - | Blood Conditions * bleeding from the lungs or intestines caused by typhoid or dysentery * disturbance in blood circulation, |
| Viscum album | combined with Horsetail * internal and external hemorrhage * nosebleed Brain and Nervous System Conditions * convulsions |
| | * convulsive nervous disorders * delerium * dizziness * epilepsy * headache * hysterical complaints * nerves * nervous |
| | debility * neuralgia * St. Vitus's Dance * weakened state of the Nervous System Cardiovascular Conditions * arteriosclerosis |
| | * hardening of the arteries * heart disease (lessens reflex irritability while raising the frequency of a slow pulse) * heart and |
| | circulatory complaints * heart flutters * normalizes circulation * poor circulation * prevention and treatment of strokes * |
| | raises low blood pressure, combined with Yarrow, Rosemary and Hawthorn * reduces high blood pressure, combined with |
| | Hawthorn, Horsetail and Garlic * strengthens the heart It is used for the side effects of abnormal blood pressure, such as: * |
| | blood rushing to the head * buzzing in the ear * dizziness * visual defects Female Conditions * bleeding after childbirth * |
| | chronic cramping * female infertility * heavy menstruation * uterine and menstrual disorders It is also helpful for women |
| | during menopause for: * breathing difficulties * feelings of anxiety * heart palpitation * hormonal imbalance * hot flushes * |
| | normalizing circulation Gastrointestinal Conditions * faulty digestive processes Glandular Conditions * benefits the whole |
| | glandular system * favorably influences the pancreas * stimulates glandular activity relating to digestion Gastrointestinal |
| | Conditions * debility of the digestive system. combined with Cayenne Genitourinary System Conditions * urinary disorders |
| | Inflammatory Conditions * arthritic pain Metabolic Conditions * aids metabolism * chronic metabolic disorders * diabetes |
| | Other * cancer * lack of energy and unwillingness to work It is used externally for: * chill blains * frost bite * sores * ulcers * |
| | varicose veins |
| Mugwort - | Brain and Nervous System Conditions * epilepsy * fits * hysteria * insomnia * nervousness * palsy * shaking Gastrointestinal |
| Artemesia | Conditions * digestive aid * disorders of the stomach * promotes proper digestion * stimulates the appetite * weakness of the |
| vulgaris | stomach Female Conditions * induces menses (periods), especially combined with Cramp Bark, Pennyroyal or Southernwood |
| | * obstruction of menstruation * regulates menstruation Inflammatory Conditions * rheumatism Liver Conditions * beneficial |
| | effect on bile production * jaundice * stimulates the liver Respiratory System Conditions * bronchitis * colds Spleen |
| | Conditions * obstructions of the spleen Other * antidote for opium * headache * intermittent fevers Externally: Used with |
| | Agrimony and Chamomile to ease: * gout * rheumatism * sore aching muscles * tired legs * to treat the irritation from poison |
| | oak |
| Myrrh - | Bacterial, Viral and Fungal Conditions * Candida/Thrush * destroys bacteria it comes into contact with * herpes Blood |
| Commiphora | Conditions * increases the number of white blood cells Cardiovascular Conditions * increasing circulation Female Conditions |
| myrrha | * excessive bleeding during menstruation, combined with Aloe vera Gastrointestinal Conditions * constipation, especially |
| | when combined with Aloe vera * used as a tonic in dyspepsia Myrrh is good for: * bad breath Immune System Conditions * |
| | stimulates the immune system Respiratory System Conditions * asthma * bronchial and other chest problems * chest |
| | problems * chronic catarrh * coughs * disinfects the secretions of bronchial mucous membranes * inflamed throat * tonsillitis |
| | * ulcerated mouth, throat and tongue Other * bad legs * debility, combined with Aloe vera * sore or loose teeth * spongy |
| | gums * ulcers Externally Myrrh is useful for: * leucorrhea * a local disinfectant * abscesses * boils * cold sores (early stage) * |
| | foul ulcers * skin infections * sores and wounds * fighting and preventing gum disease * gingevitis * sore or loose teeth * |
| | spongy gums * weak gums |
| Neem - | Bacterial, Viral and Fungal Conditions * AIDS * fungal infections * malaria * mononucleosis * sexually transmitted diseases |
| Azadirachta | * shingles * yeast infections Blood Conditions * blood disorders Brain and Nervous System Conditions * headache * nerve |
| indica | disorders Cardiovascular Conditions * heart diseases Gastrointestinal Conditions * digestive problems Genitourinary Tract |
| | Conditions * kidney problems Immune System Conditions * allergies * strengthens the immune responses of the body's |
| | overall immune system Liver Conditions * hepatitis Metabolic Conditions * diabetes Respiratory System Conditions * colds * |
| | influenza * sore throat Reproductive System Conditions * birth control remedy for both men and women Skin Conditions * |
| | acne Other * cancer * earache * fatigue * fever * food poisoning * periodontal diseases * ulcers Externally it is used to treat: |
| | * acne * bruises * burns * skin diseases * sprains * ulcers * wounds |
| Nettles - Urtica | Blood Conditions * anemia * blood in the urine * hemorrhages or blood loss * hemorrhages of the uterus * hemorrhoids |
| urens, Urtica spp. | Cardiovascular Conditions * high blood pressure * improves circulation Female Conditions * excessive menstrual flow * |
| | menstrual difficulties * premenstrual syndrome * promotes milk flow in nursing mothers * support during pregnancy |
| | Gastrointestinal Conditions * diarrhea * stimulates the digestive system Genitourinary Tract System Conditions * gout * |
| | improves kidney function * urinary tract problems Glandular Conditions * stimulates the pancreas in the control of blood |
| | sugar levels Inflammatory Conditions * reduces susceptibility to rheumatic complaints * rheumatism Liver Conditions * liver |
| | problems Male Conditions * enlarged prostate (often combined with Saw Palmetto and Willow Herb) Metabolic Conditions * |
| | diabetes Respiratory System Conditions * asthma * clears breasts and lungs of all mucous * coughs * hay fever and allergies |
| | * prevents colds * shortness of breath Skin Conditions * eczema * nettle rash * psoriasis Other * edema * fatigue * muscular |
| | pains * restores vitality * scurvy Externally: * applied to burns * applied to the scalp for loss of hair to stimulate hair growth * |
| | as a hair rinse to treat dandruff, combined with Rosemary * for circulation to the scalp * arthritis * neuralgia |
| Oats - Avena | Brain and Nervous System Conditions * acts as a stimulant of the motor ganglia, increasing the excitability of the muscles * |
| sativa | insomnia * nerve tonic * nervous prostration and exhaustion after febrile diseases * restores and strengthens the brain * |
| | restores the nervous system Cardiovascular Conditions * beneficial action on the heart muscle * restores and strengthens the |
| | heart Gastrointestinal Conditions * dyspepsia * easy digestion in inflammatory conditions and fevers * gastroenteritis * |
| | gastrointestinal inflammation Genitourinary System Conditions Oat Straw works very effectively on the Urinary Tract and is |
| | beneficial for adverse conditions of the: * bladder * kidneys * ureters and * spasmodic conditions of the bladder and ureters * |
| | uterine tonic Male Conditions * beneficial for the prostate gland * impotence * spermatorrhea (involuntary release of semen) |
| | Reproductive System * produces a tonic effect on the nerve structure of the sexual organs * sexual debility due to |
| | overindulgence Respiratory Conditions * chest problems Viral Conditions * herpes * shingles Other * bone-building * |
| | exhaustion * fevers * recovering from an illness or debilitating condition * supplying necessary fiber in the diet Externally: |
| | Oats are used to clear * acne * skin disorders * gout * gravel * kidney problems * liver ailments * lumbago * paralysis * |
| | rheumatic problems * abdominal problems * bladder disorders * intestinal colic * abdominal problems * bedwetting * bladder |
| | problems * intestinal colic * chronically cold feet * tired feet * chill blains * eye problems * flaky skin * frostbite * skin |
| | diseases * wounds |
| Olive - Olea | Blood Conditions * stabilizes blood sugar levels Bacterial, Fungal, Viral or Parasitic Conditions Eradicates parasites |
| europaea | including: * giardia * intestinal worms * malaria forming protozoa * microscopic protozoa * pinworms * rectal worms * |
| | ringworm * roundworm * tapeworms Helps inhibit and prevent infection by viruses and retroviruses: * chicken pox * cold |
| | sores * genital herpes * genital warts * herpes Type 1 * HIV virus * improves symptoms of chronic fatigue syndrome and |
| | related conditions * lupus * meningitis (viral) * mononucleosis * shingles * warts Helps inhibit and prevent infection by |
| | bacteria: * anthrax * chlamydia * gastric ulcers caused by H. pylori * gonorrhea * meningitis (bacterial) * salmonella * |
| | staphylococcal food poisoning * syphilis * toxic shock syndrome * trichonomas * urinary tract infections * vaginitis Helps |
| | inhibit and prevent infection by fungi, yeasts and moulds: * chronic toenail fungus infection * eradicates candidiasis |
| | (Candida) and other yeast conditions * vaginitis Olive is also used for: Brain and Nervous Conditions * nervous tension |
| | Cardiovascular Conditions * boosts energy levels * barium chloride and calcium induced arrhythmia * enhances the elasticity |
| | of arteries * healthy dilatory action in coronary arteries * improves blood flow * reduces high blood pressure * vasodilator |
| | effect on the smooth layer of coronary arteries Gastrointestinal Conditions * constipation in children * habitual constipation * |
| | increases peristalsis * lead colic Genitourinary Conditions * bladder infections Immune System Conditions * autoimmune |
| | disorders Inflammatory Conditions * rheumatic fever * meningitis * relieves inflammation caused by arthritis and rheumatoid |
| | arthritis Liver Conditions * dispels biliary concretions * dissolves cholesterin (the main ingredient of gallstones) * dissolves |
| | cholesterol * Hepatitis A, B, C * increases bile secretions Respiratory Conditions * abdominal chill * chest complaints * helps |
| | prevent influenza * pneumonia * sinus infections * streptococcus infection in throat Other * antidote to irritant poisons * |
| | disperser of acids * ear infections * fevers * fibromyalgia * fights free radical production * food poisoning * soothes mucous |
| | membranes * rabies Externally it relieves: * toothache * hemorrhoids * athlete's foot * fungus in nails* bruises * burns * |
| | crusts and scales on the body * cutaneous affections * dry cuticles and nails * dry skin * engorged breasts * hair care * insect |
| | bites * muscular and joint complaints * protects the skin surface from air * skin inflammations * sprains * stings * wounds |
| Paeonia - Paeonia | Blood Conditions * nourishes the blood Brain and Nervous System Conditions * assists memory * epilepsy (often combined |
| lactiflora | with Licorice) * smooth muscle spasm in migraine headaches Cardiovascular Conditions * smooth muscle spasm in angina |
| | Female Conditions * dysmenhorrea * menstrual dysfunction * leukorrhea * polycystic ovary syndrome (often combined with |
| | Dong Quai or Licorice) * smooth muscle spasm in dysmenhorrea Gastrointestinal Conditions * dysentery Muscular |
| | Conditions * muscle cramping Other Conditions * blurred vision * dizziness * spontaneous sweating |
| Passionflower - | Brain and Nervous System Conditions * anxiety * debility with pain * epilepsy * hyperactivity * hysteria * insomnia * |
| Passiflora | irritability * muscle tension due to anxiety * narcotic induced hangover * nervous headache * nervous tension * neuralgia * |
| incarnata | pain of shingles * Parkinson's Disease * restlessness * sedative for nervous conditions * spasms Cardiovascular Conditions * |
| | increases rate of respiration without unduly affecting circulation * slightly reduces arterial pressure Female Conditions * |
| | dysmenorrhea (painful menstruation) Gastrointestinal Conditions * diarrhea * dysentery Respiratory System Conditions * |
| | asthma where there are severe spasms Other * eye infections * eye strain |
| Pau D'arco - | pain * arthritis * inflammation of the prostate gland (prostatitis) * fever * dysentery * boils and ulcer * various cancers. * pain |
| Tabebuia spp. | * arthritis * inflammation of the prostate gland (prostatitis) * fever * dysentery * boils and ulcer * various cancers. |
| Peppermint - | Brain and Nervous System Conditions * eases anxiety and related conditions * hysteria and nervous disorders, especially |
| Mentha piperita | when combined with Wood Betony * headache and vomiting due to nervous causes * insomnia * migraine * nervousness |
| | Cardiovascular Conditions * heart palpitations Female Conditions * vomiting during pregnancy Gastrointestinal Conditions * |
| | abdominal pains * colitis * diarrhea * dysentery * flatulent colic * heartburn * poor digestion * sedative on the stomach * |
| | soothes the digestive system * stimulates digestive secretions * strengthens the bowel * sudden pains and cramps in the |
| | abdomen Liver Conditions * stimulates bile secretions Respiratory System Conditions * alleviates the feverish symptoms of a |
| | cold, especially when combined with Elder Flowers and Yarrow chills and colds for children * eases a clogged head in colds |
| | and influenza Other * in early indication of disease * nausea * seasickness * cleans and strengthens the body * induces |
| | perspiration * raises internal heat * with bitter herbs to improve their taste * with purgatives to prevent griping |
| | Gastrointestinal Conditions * colic * cramps in the abdomen * diarrhea * dyspepsia * flatulence (gas/wind) * stomach ache |
| | Other * a local anesthetic * cholera * sickness and nausea * sudden pains * toothache Respiratory Conditions * bronchitis * |
| | chest complaints * colds * laryngitis * nasal catarrh * lumbago * neuralgia * rheumatism Externally to relieve * itching * skin |
| | conditions |
| Phyllanthus | Liver Conditions * Hepatitis B * jaundice * may have a support role in Diabetes Viral Conditions Viral liver diseases |
| amarus | including: * acute hepatitis * chronic persistent hepatitis * part of the treatment for chronic active hepatitis |
| Picrorrhiza | Immune System Conditions * acute and chronic infections * treatment for allergies * treatment for autoimmune disorders * |
| kurroa | weakened immunity Liver Conditions * liver infections * toxic liver damage Respiratory Tract Conditions * asthma |
| Plantain - | Brain and Nervous System Conditions * epilepsy * headaches Digestive Conditions * bowel complaints * diarrhea * |
| Plantago major | dysentery * gastrointestinal ulcers * stomach ulcers Genitourinary Conditions * bladder infections * disorders of the kidneys * |
| | obstructions of the kidneys Liver Conditions * intermittent fevers * jaundice * obstructions of the liver Parasitic Conditions * |
| | parasites * worms Reproductive System Conditions * female complaints * increasing virility * promoting fertility Respiratory |
| | Conditions * asthma * bronchitis * clearing mucous from the body * colds * coughs * lung problems in children * ulceration |
| | of the lungs Other * lethargy * malignant ulcers * neutralizing poisons * obstructions of the spleen * toothache (root chewed) |
| | * ulceration of the mouth and throat * vomiting Externally it is used for * bruises * burns and scalds * cooling inflammation |
| | of the eyes * earache and deafness * hemorrhoids (piles) * inflammation of the skin * nettle rash * ringworm * scabs and |
| | itches * shingles * sores * stings and insect bites stopping bleeding of minor surface external wounds * treating wounds * |
| | tumors * arthritis * gout * leucorrhea |
| Polygala | Brain and Nervous System Conditions Polygala tenuifolia benefits the mental states * amnesia * anxiety, often combined with |
| tenuifolia - | the herbs Kava, Passionflower, Schisandra and Zizyphus * constrained emotions * dream-disturbed sleep, often combined |
| Polygala | with the herb Schisandra * excessive brooding * fear, often combined with the herb Schisandra * forgetfulness * insomnia, |
| tenuifolia | often combined with the herb Schisandra * mental disorientation * neuresthenia, often combined with the herb Schisandra * |
| | palpitations, often combined with the herbs Kava, Passionflower, Schisandra and Zizyphus * pent-up emotional states * |
| | restlessness Respiratory Tract Conditions Often combined with the herbs Adhatoda, Grindelia, Euphorbia and Elecampane for |
| | Catarrhal Respiratory states such as: * asthma * bronchitis * common cold as well as * acute and chronic lower respiratory |
| | tract infections * allergic respiratory disorders, often combined with the herbs Albizia, Baical Skullcap * cough with copious |
| | sputum * sputum difficult to expectorate Skin Conditions * boils * sores * swellings |
| Polygonum | Blood Conditions * high blood cholesterol * tones the vital essence and blood Brain and Nervous System Conditions * |
| multiflorum - | blurred vision * dizziness with tinnitus * epilepsy * neurasthenia especially with insomnia * neuritis * schizophrenia |
| Polygonum | Muscular and Skeletal Conditions * knee pain * lower back pain * fortifies muscles, tendons and bones * numbness of limbs |
| multiflorum | Other * premature aging * premature greying * promotes longevity * tonic for elderly * weak connective tissue Action: |
| | astringent [a binding agent that contracts organic tissue, reducing secretions or discharges of mucous and * fluid from the |
| | body] * bitter [applied to bitter tasting drugs which act on the mucous membranes of the mouth and stomach to increase |
| | appetite and promote digestion] * sweet * warm |
| Queen's Delight - | Bacterial Conditions * primary and secondary syphilis, especially combined with Sarsaparilla Blood Conditions * impure |
| Stillingia | conditions of the blood Female Conditions * leucorrhea Glandular Conditions * scrofula affections Liver Conditions * hepatic |
| sylvatica | affections Respiratory Conditions * chronic bronchitis * laryngitis Other * influences the secretory functions * skin diseases |
| Raspberry - | Raspberry Leaf is one of the most proven female herbs as it * eases menstrual cramps * has an astringent action on the female |
| Rubus Idaeus | reproductive tract * helps a tired uterus as a result of childbearing * improves the efficiency of the muscle of the womb * |
| | prevents or overcomes prolapse of the uterus in older women * regulates menstrual flow * relieves leucorrhea * tightens, |
| | tones and strengthens the ligaments supporting the uterus During pregnancy and labor it: * cuts down on post-partum bleeding |
| | * decreases uterine swelling after childbirth * helps prevents miscarriage * helps the womb return to its normal place after |
| | pregnancy * helps to reduce false labor pains * improves the efficiency of contractions * increases milk supply in nursing |
| | mothers * reduces the pain of childbirth * reduces the labor time during childbirth * strengthens and tones the tissues of the |
| | uterine wall during pregnancy to help with contractions and hemorrhaging * strengthens the pelvic muscles and ligaments to |
| | promote an easy normal birth * is used in the last trimester of pregnancy as a uterine tonic Gastrointestinal Conditions * |
| | diarrhea * diarrhea in infants * dysentery * extreme laxity of the bowels * stomach complaints in children Other * allays thirst |
| | * all loose conditions * fevers * soothes mucous membranes * vomiting in small children *Externally it is used for: * bleeding |
| | gums * canker of the mouth * dissolving tartar on the teeth * inflammation of the mouth * sore throat * ulcers of the mouth * |
| | burns * proud flesh * scalds * skin rashes * sores * ulcers * wounds * burns * cleansing wounds * promoting healing * |
| | removing proud flesh * scalds |
| Red Clover - | Blood Conditions * cleanses blood (by promoting urine and mucous production improving circulation, and stimulating the |
| Trifolium | secretion of bile) * improves the blood's hemoglobin levels and the size and number of blood platelets * vital blood tonic |
| pratense | Female Conditions * mastitis (inflammation of the breast) * menopausal symptoms (the isoflavones in Red Clover help with |
| | menopausal symptoms such as: * bone loss associated with osteoporosis * cardiovascular health * hot flashes * relieves |
| | leucorrhea Gastrointestinal Conditions * alkaline properties Genitourinary Conditions * cleansing action through the kidneys |
| | Liver Conditions * aids detoxification * jaundice * stimulates the secretion of bile Muscular Conditions * joint disorders |
| | Nervous System Conditions * quietens the nerves (a nerve tonic) * sedative for exhaustion Respiratory Conditions Red Clover |
| | has a strong antispasmodic effect on: * asthma * colds * hoarseness * bronchitis * coughs in children * disorders of the |
| | respiratory organs * irritation of the vocal cords, windpipe and bronchial tubes * spasmodic coughing * wheezing * whooping |
| | cough Skin Conditions Red Clover is an excellent skin remedy in cases of: * acne * eczema * psoriasis * rashes * skin |
| | disorders Other Conditions * beneficial for the organs of elimination - lungs, kidneys, bowels and skin * cancer, in particular |
| | breast cancers * promotes eliminatory functions * strengthens children with weak systems * tumors Externally Red Clover is |
| | used as a: * salve * ointment * athlete's foot * cancerous growths |
| Rehmannia | Blood Conditions * breaks up congealed blood * hyperlipidemia (in conjunction with other treatments) * invigorates blood |
| glutinosa | Female Conditions * amenorrhea due to blood stasis Gastrointestinal Conditions * abdominal pain * atonic dyspepsia (in |
| | small doses) * bacillary dysentery * constipation * diarrhea caused by an irritating body in the intestines * dysentery * |
| | enteritis * gastrointestinal hemorrhage * increases the flow of saliva (when chewed) * stimulates appetite and digestion (cold |
| | extract in small doses) * torpor and debility of the digestive organs (in small doses) Genitourinary System Conditions * early |
| | stages of chronic renal failure (in conjunction with other agents) Glandular Conditions * acute pancreatitis (in conjunction |
| | with other agents) Liver Conditions * acute hepatitis (in conjunction with other agents) * acute cholecystitis * jaundice due to |
| | damp heat Other Conditions * cancer * fevers * as a laxative for constipation - higher end of dosage range * as an astringent |
| | for diarrhea - lowest end of dosage range |
| Rheum palmatum | Blood Conditions * breaks up congealed blood * hyperlipidemia (in conjunction with other treatments) * invigorates blood |
| | Female Conditions * amenorrhea due to blood stasis Gastrointestinal Conditions *abdominal pain * atonic dyspepsia (in |
| | small doses) * bacillary dysentery * constipation * diarrhea caused by an irritating body in the intestines * dysentery * |
| | enteritis * gastrointestinal hemorrhage * increase the flow of saliva (when chewed) * stimulates appetite and digestion (cold |
| | extract in small doses) * torpor and debility of the digestive organs (in small doses) Genitourinary System Conditions * early |
| | stages of chronic renal failure (in conjunction with other agents) Glandular Conditions * acute pancreatitis (in conjunction |
| | with other agents) Liver Conditions * acute hepatitis (in conjunction with other agents) * acute cholecystitis * jaundice due to |
| | damp heat Other Conditions * cancer * fevers * as a laxative for constipation - highest end of dosage range * as an astrigent |
| | for diarrhea - lower end of dosage range |
| Ribwort - | Blood Conditions * coagulates blood * rids the blood of toxins * weak or bad blood * diarrhea * gastritis * enteritis |
| Plantago | Genitourinary Tract Conditions * bladder disorders * cystitis where is bleeding * diseases of the bladder, combined with |
| lanceolata | Thyme * stones * weak kidneys Glandular Conditions * goiter * malignant glandular disorders Liver Conditions * liver |
| | disorders Parasitic Conditions * worms Respiratory Conditions * bronchial asthma * bronchitis * chronic catarrh * coughs |
| | and irritation from coughing * disorders of the respiratory system * hoarseness, with slight cough * loosens mucous and slime |
| | in the inner organs * mucous congestion * rids the lungs of phlegm * soothes inflamed mucous membranes * tuberculosis of |
| | the lungs * weak lungs * whooping cough It is used externally for: * cuts * dog bites * eczema * hemorrhoids * herpes * |
| | insect bites * malignant growths * open sores on stumps of amputees * scratches sores * stings from poisonous insects * |
| | thrombosis * ulcers * wounds * blisters |
| Rosehips - Rosa | Blood Conditions * blood purifier * conditions where blood is being brought up via the lungs * internal hemorrhaging * |
| canina | spitting of blood Female Conditions * disorders of the breast * excessive menstruation * uterine cramps Gastrointestinal |
| | Conditions * diarrhea * dysentery * strengthens the stomach Respiratory Conditions * chest infections * colds * coughs * |
| | influenza Urinary Tract Conditions * cleans out the Kidneys and Bladder * eliminates uric acid accumulations, helping with |
| | gout and rheumatic complaints * fluid retention Other * allays thirst * builds strength when convalescing * deficiency of |
| | Vitamin C in children * general debility and exhaustion Externally * nourishes the skin The common Red Rose is also used |
| | medicinally The petals are used for: Bacterial Conditions * sharpness and heat of urine in gonorrhea Blood Conditions * |
| | blood purifier Brain and Nervous System Conditions * dizziness * headache Cardiovascular Conditions * heart and nerve |
| | tonic Respiratory Tract Conditions colds coughs sore throat (Rose honey) Other * invigorates the tired body * mouth sores * |
| | toothache Female Conditions * menstrual complaints Genitourinary Conditions * soreness of the bladder * uterine cramps |
| | Externally for: * chafed hands and lips * sores of the mouth, ears and anus * ulcers |
| Rosemary - | Brain and Nervous System Conditions * general debility after long-term nervous or physical illness * improves the memory * |
| Rosemarinus | insomnia * mental fatigue * nervous anxiety and tension * nervous depression * nervous disorders * restorative effect on the |
| officinalis | nervous system * soothes the nerves * stimulates the brain and nervous system * tension headaches * migraines |
| | Cardiovascular Conditions * improves circulation * raises blood pressure * stimulates the circulatory system * weak heart |
| | subject to palpitation (in small doses) Gastrointestinal Conditions * bad breath * upset stomachs * promotes proper digestion |
| | * toning and calming effect on the digestion Genitourinary Conditions * dropsy Female Conditions * regulates the menstrual |
| | cycle Liver Conditions * promotes liver function * promotes the production of bile Reproductive System Conditions * |
| | stimulates the sexual organs Respiratory System * colds * colic Other * eases cramps * expels morbid matter from the system |
| | * failing eyesight * headache* bites * stings * as a decongestant * as an inhalant * for exhaustion * for headaches * to |
| | enhance memory and clear concentration * arthritis * bruises * eczema * gout * muscular pain * neuralgia * revitalizing |
| | paralyzed limbs * rheumatism * rheumatoid arthritis * sciatica * scrofulous sores * wounds and rubbed into hair * for |
| | stimulating the hair bulbs to renewed activity * to prevent premature baldness |
| Rue - Ruta | Blood Conditions * strengthens the capillary veins Brain and Nervous System Conditions * acute pains in the head * anxiety |
| graveolens | * convulsions * epilepsy * fainting spells * hysterical affections * nervous heart problems * nervousness * spasms * tension |
| | headaches * vertigo Cardiovascular Conditions * arteriosclerosis, combined with Hawthorn, Horsetail, Mistletoe and |
| | Shepherd's Purse * increases peripheral circulation * lowers high blood pressure * venous congestion Eyesight * makes the |
| | sight both sharp and clear * preserves of sight Female Conditions * congestion of the uterus * heart palpitations in |
| | menopausal women * painful menstruation * promotes the onset of menstruation Gastrointestinal Conditions * abdominal |
| | troubles * colic * cramps in the bowels * flatulence (gas/wind) * improves appetite and digestion * stomach cramps |
| | Genitourinary Conditions * relieves gouty pains Inflammatory Conditions * relieves rheumatic pains Parasitic Conditions * |
| | eliminates worms Respiratory Conditions * colic * croup * coughs * difficult breathing * head congestion Other * nausea * |
| | tendonitis (internally and externally) * wards off fever Rue is used in small amounts to expel poisons from the system, such as |
| | from bites of: * snake * scorpion * spider * jellyfish Externally Rue is used for: * gout * pains in the joints * rheumatism * |
| | sciatica * warts |
| Sage - Salvia | Bacterial and Viral Conditions * retro viruses, e.g. Herpes, HIV, etc. * staphylococcus infections Brain and Nervous System |
| officinalis | Conditions * aids the memory * cramps * delerium associated with fevers * depression * disorders of the spinal cord * cases |
| | mental exhaustion * headaches (combined with Peppermint, Rosemary, and Wood Betony) * nervous conditions * nervous |
| | excitement, associated with brain or nerve diseases * paralysis * strengthens the powers of concentration * trembling of limbs |
| | *vertigo * weak nerves Cardiovascular Conditions * prevention of strokes Female Conditions * amenorrhea (irregular |
| | menstruation) * decreasing milk flow in lactating women during weaning * dysmenorrhea (painful menstruation) * |
| | hemorrhage from the lungs or stomach * leucorrhoea * night sweats and hot flushes during menopause, combined with |
| | Horsetail and Valerian * regulates the menstrual cycle * reduces perspiration (because of its estrogenic properties) * tonic |
| | after childbirth Gastrointestinal Conditions * abdominal troubles * all stomach troubles * diarrhea * eliminates mucous |
| | congestion in the stomach * enteritis * flatulence (gas) * gastritis * increases the appetite * rectifies intestinal troubles * |
| | stimulates the digestive system * weak stomach Genitourinary Conditions * kidney troubles Glandular Conditions * glandular |
| | disorders Liver Conditions * biliousness * liver complaints Respiratory Tract Conditions * colds * dispels phlegm from the |
| | respiratory organs and stomach * eliminates mucous congestion in the respiratory passages and stomach * influenza Sage is a |
| | good for: * laryngitis * sore throat * tonsillitis * ulcerated throat Chewing the fresh leaves soothes: * sore throats * mouth |
| | sores Other * a deodorant Sage is a good for: * inflammation of tooth pulp * loose and bleeding teeth * preventing excessive |
| | flow of saliva * ulcerated or receding gums * ulcerated gums and mouth Externally: * as a hair rinse to remove dandruff * for |
| | insect bites * for itchy skin * for skin eruptions * for treating sores * to stop bleeding in all cuts |
| Salvia | Blood Conditions * blood stimulant * blood stasis remover with sedative action Brain and Nervous System Conditions * |
| militiorrhiza - | increased cerebrovascular circulation * insomnia * nerve compression * diabetic neuropathy Cardiovascular Conditions |
| also known as | Buerger's disease (blood vessel disease resulting in poor circulation in the lower legs, causing skin problems such as * burning |
| Dan Shen | * tingling * ulcerations * ischemic heart disease * angina * heart attacks * high blood pressure * increased peripheral |
| | circulation * palpitations * pulmonary fibrosis Female Conditions * menstrual problems Glandular Conditions * adjunct to |
| | treatment or lymphoma (a cancer that begins in the lymphatic system) Immune Conditions * autoimmune diseases * |
| | scleroderma Liver Conditions * acute and chronic liver disease Skin Conditions * acne * other skin disorders |
| Sarsaparilla - | Bacterial and Viral Conditions * retro-viruses, e.g. herpes, HIV etc. * syphilis Genitourinary Conditions * gout Inflammatory |
| Smilax ornata | Conditions * rheumatism Respiratory Tract Conditions * coughs and colds The smoke of Sarsaparilla is used for: * asthma |
| | Other * abscesses * ulcers Externally, Sarsaparilla is used for: * abscesses * rheumatism * skin diseases * ulcers |
| Saw Palmetto - | Gastrointestinal Conditions * improves digestion * increases the assimilation of nutrients to the body * stimulates appetite |
| Serenoa serrulata | Genitourinary Conditions Saw Palmetto is called the ‘plant catheter’ due to its therapeutic effect on the neck of the bladder and |
| | is used to: * benefit the urinary tract * relieve strain from painful bladder problems * tone the bladder by improving urinary |
| | flow Regular use of Saw Palmetto may decrease urinary frequency, especially at night by: * allowing complete bladder |
| | expulsion * reducing inflammation of the bladder Brain and Nervous System Conditions * nourishes the nervous system |
| | Glandular System * aids the thyroid in regulating sexual development * for those suffering from thyroid deficiency * marked |
| | effect on glandular system Female Conditions * aphrodisiac for women, combined with Damiana * atrophy of mammary |
| | glands * balances the hormones * for reduced or absent libido (sex drive) * frigidity * increases the nutrition of the mammary |
| | glands in functional atony of these organs * ovarian and uterine irritability * stimulates breast enlargement and lactation |
| | Inflammatory Conditions * decreases catarrh in the mucous membranes Male Conditions * aphrodisiac for men, combined |
| | with Damiana * atrophy of testes * balances the hormones * benefits the prostate gland * for reduced or absent libido (sex |
| | drive) * therapeutic effect on the prostate gland * enlarged or weakened prostate gland (often combined with Willow Herb) * |
| | reducing inflammation of the bladder and enlarged prostrate * impotence * increases the nutrition of the testicles in functional |
| | atony of these organs Reproductive System * normalizes activity of the reproductive glands and organs Respiratory |
| | Conditions * asthma * clears chest congestion * chronic bronchitis * conditions associated with colds * coughs * mucous |
| | congestion Other * for all wasting diseases * general tonic during convalescence from illness as it builds up strength and |
| | increases flesh rapidly * in the treatment of diseases that deprive the body of strength and growth * tissue builder |
| Senna - Cassia | Gastrointestinal Conditions * as a cleanser during a fast (best combined with Cinnamon, Fennel or Ginger) as a very effective |
| spp., Cassia senna | laxative * constipation * eases nausea and biliousness * halitosis * increases peristaltic movements of the colon (by local |
| | action on the intestinal wall) Senna works by irritating the lining of the upper intestines which provokes reflex muscular |
| | activity in the colon resulting in a bowel motion (due to the chemical anthraquinone) Liver Conditions * eases biliousness |
| | Parasitic Conditions * cleanses the system of worms |
| Shepherd's Purse - | Cardiovascular Conditions * high or low blood pressure * normalizies circulation, as Shepherd's Purse is a circulation |
| Capsella bursa- | equalizing herb * regulates heart action Female Conditions * excessive menstrual flow * facilitates childbirth * menopause * |
| pastoris | painful menstruation, combined with Rue and Rosemary * promotes uterine contraction during childbirth * regulates |
| | menstruation during puberty * stops post natal bleeding * uterine cramp Externally: * nursing mothers with swollen breasts |
| | (compress) Blood Conditions * bleeding from the kidneys, combined with Horsetail * bleeding from the lungs * bleeding |
| | hemorrhoids * hematuria * intestinal bleeding * nosebleeds * passive hemorrhages from mucous membranes * stomach |
| | hemorrhage * uterine bleeding * wounds which will not stop bleeding Gastrointestinal Conditions * chronic diarrhea * colic * |
| | dysentery * promotes bowel movements through intestinal contraction Genitourinary Tract Conditions * abscesses of the |
| | bladder * bedwetting in children * catarrhal conditions of the bladder and ureters * increases the flow of urine * irritation of |
| | the urinary tract caused by uric acid or insoluble phosphates or carbonates * kidney complaints * ulcerated conditions of the |
| | bladder * urine with white mucous discharge Externally Shepherd's Purse is used for: * bruises * external muscular disorders |
| | * limb or muscular atrophy * rheumatic joints * strains |
| Skullcap - | Brain and Nervous Conditions * convulsions * delerium tremens (in alcoholics) * food for the nerves * excitability * |
| Scutellaria | hydrophobia * hysteria * insomnia and restlessness * lessens the affects of epilepsy (in small doses) * nervous headaches * |
| laterifolia | neuralgia * rickets * spasms * supports and strengthens as well as giving immediate relief from all chronic and acute diseases |
| | that affect the nerves * St. Vitus Dance (soothes the nervous excitement and induces sleep when necessary) Female |
| | Conditions * feminine cramps * menstrual troubles Respiratory Conditions * headache arising from incessant coughing and |
| | pain Inflammatory Conditions * rheumatism Reproductive Conditions * regulates sexual desires Other * reduces fevers |
| Slippery Elm - | Brain and Nervous System Conditions * induces sleep (if taken at night) Cardiovascular Conditions * heart remedy, combined |
| Ulmus fulva | with Bugleweed Gastrointestinal Conditions * diarrhea, combined with Bayberry, Skullcap and Myrrh * dysentery * enteritis |
| | * gastric catarrh * gastritis * gastrointestinal irritation * irritation of the mucous membranes of the stomach and intestines * |
| | mucous colitis * puts a protective mucilaginous lining over the stomach and esophagus * reduces over-activity in the bowel, |
| | via a nerve reflex action * neutralizes stomach acids Genitourinary Conditions * cystitis * irritation of the urinary tract |
| | Glandular Conditions * boosts the adrenal glands Inflammatory Conditions * soothes all mucous membranes Respiratory |
| | Conditions * bleeding from the lungs * bronchitis * chest disorders * coughs * irritation of the pharynx * soothes coughs * |
| | soothing to the whole respiratory system * sore throat Other * builds up the body and prevents wasting * draws out impurities |
| | * heals all parts of the body * nutritive food for children, invalids and convalescents * soothing after the discomfort of |
| | vomiting * used in all cases of weakness Externally it is used for: * abscesses * all inflamed surfaces * boils * burns * chill |
| | blains * congestion * eruptions * gangrenous wounds * poison ivy * purulent ophthalmia * skin cancers * skin diseases * |
| | suppurationsswollen glands * ulcers * wounds |
| Squaw Vine - | Brain and Nervous System Conditions * natural nerve sedative (usually in combination with Raspberry Leaves) Female |
| Michella repens | Conditions * all uterine complaints * amenorrhea (irregular menstruation) * dysmenorrhea (painful menstruation) * helps |
| | prevent miscarriage * in childbirth (taken for the last 3 weeks before birth) * menorrhagia (excessive or prolonged |
| | menstruation) * relieves congestion of the uterus and ovaries * strengthens the uterus * vaginal infections * very beneficial for |
| | women Externally: Squaw Vine * can be rubbed on the nipples when sore Gastrointestinal Conditions * diarrhea |
| | Genitourinary Conditions * dropsy * suppression of urine |
| St John's Wort - | Blood Conditions * anemia * internal bleeding * spitting of blood, combined with Knotgrass Brain and Nervous Conditions |
| Hypericum | St. John's Wort has an affinity for nerve endings and is one of the best herbs for mood elevation and has been used for: * |
| perforatum | anxiety * depression * exhaustion * headache * helplessness * hopelessness * hysteria * insomnia or restless sleep * long- |
| | term nervous debility and fatigue * melancholy * mild to moderate depression * nervous complaints * nervous debility * |
| | neuritis * neurosis * repairing nerve damage * sadness * shingles * sleepwalking * speech disorders * tension * trigeminal |
| | neuralgia Female Conditions * menopausal symptoms, especially where anxiety is pronounced (combined with Vervain and |
| | Oatstraw) * menstrual cramping * menstrual difficulties * promotes regular menstruation * uterine cramping Gastrointestinal |
| | Conditions * colic * constipation * dysentery * gastric diarrhea * nausea * intestinal problems * soothes the digestive system |
| | * stomach ache * vomiting, combined with Knotgrass Genitourinary Tract Conditions * affections of the urinary passages * |
| | bedwetting in children * bladder troubles * incontinence * suppression of urine Inflammatory Conditions * arthritis * catarrh |
| | * reduces pain and inflammation Liver Conditions * favorable action on the secretion of bile * jaundice * liver remedy, |
| | combined with Aloe vera Respiratory Conditions * bronchitis * congestion of the lungs * coughs and colds * lung diseases |
| | generally * mucous obstructions in chest and lungs Viral Conditions St. John's Wort's potent antiviral and antibacterial |
| | properties are used for: * herpes simplex * influenza viruses * treatment for acquired immune deficiency [AIDS] Other * |
| | sciatica * ulcers Externally: is used for: * abdominal pain in babies * abscesses * bad insect stings * bruises * closes wounds * |
| | contusions * cuts and grazes * dirty, septic wounds, * glandular swellings * healing wounds * injured nerves * injuries caused |
| | by a blow * open wounds * preventing wounds, scratches, cuts and punctures from becoming septic * relieving inflammation |
| | * sores * sprains * swellings * swollen lymph glands * ulcers * varicose veins Eases pain of: * lumbago * neuralgia * |
| | rheumatism * sciatica * sore back * burns * scalds * sunburn |
| St Mary's Thistle - | Brain and Nervous System Conditions * depression * severe melancholy Female Conditions * increases breast-milk |
| Silybum | production Genitourinary Conditions * breaks and expels stones * dropsy Inflammatory Conditions * catarrh Liver conditions |
| marianum | St. Mary's Thistle supports nutritionally the liver's ability to maintain normal liver function. It has been used to treat nearly |
| | every known form of liver disease including: * biliary derangements * cirrhosis * hepatitis * jaundice * liver damage due to |
| | drug and alcohol abuse * necroses * removing obstructions from liver and spleen * skin conditions related to poor liver |
| | function * stimulating the secretion of bile * the liver detoxification process It protects the liver from damage from the |
| | harmful, detrimental effects of: * alcohol * chemotherapy * drugs * environmental toxins * by fighting pollutants, preventing |
| | free radical damage and stimulating the production of new liver cells Respiratory Tract Conditions * pleurisy Other: * tinnitis |
| | (ringing in ears) combined with Ginkgo biloba |
| Thyme - Thymus | Blood Conditions * anemia * purifies blood Brain and Nervous System Conditions * insomnia Gastrointestinal Conditions * |
| vulgaris | all stomach discomfort * colic * diarrhea * flatulence * intestinal disturbances * intestinal irritation * stomach cramps |
| | Genitourinary Conditions * gout * urinary tract infections (combined with Agrimony, Golden Rod and Hawthorn) * uterine |
| | disorders Inflammatory Conditions * catarrh Respiratory System * asthma * bronchial ailments * colds * eliminates excessive |
| | mucous discharge * loosens tight phlegm * eases a dry, barking cough * promotes perspiration at the start of a cold * |
| | shortness of breath * strengthens the lungs * whooping cough Other * fevers * hardness of the spleen Externally is used for: * |
| | easing rheumatic pain 2 doses Thyme, with 3 doses Eucalyptus, 2 doses Clary Sage * hot swellings * sciatica * warts * |
| | mouth and gum infections * sore throat |
| Uva Ursi - | Female Conditions * prevents infection of the womb after childbirth * returns the womb to its natural size postpartum (after |
| Arctostaphylos | childbirth) Genitourinary Tract Conditions * bedwetting * bladder and related infections * cystitis (bladder inflammation) * |
| uva-ursi | eases pain and discomfort in acute urinary tract infections * lights infection of the mucus membranes of the urinary tract * |
| | gravel or ulceration of the kidneys and bladder * inflammatory diseases of the urinary tract * kidney infections * kidney and |
| | bladder stones * nephritis * pyelitis * reduces inflammation of the mucus membranes of the urinary tract * reduces uric acid |
| | in the system * soothes irritation to the mucus membranes of the urinary tract * strengthens and tones the urinary tract * |
| | increases flow of urine * urethritis * urinary tract infections Immune System Conditions * fights infections Metabolic |
| | Conditions * excessive sugar in the blood (as in diabetes) Respiratory Conditions * bronchitis Other Conditions * over-acid |
| | conditions Externally it is used for: * leucorrhea |
| Valerian - | Brain and Nervous Conditions * anxiety * breathlessness * epilepsy (combined with Mistletoe) * giddiness and fainting fits * |
| Valeriana | has a remarkable influence on the cerebro-spinal system. * hypochrondriasis (abnormal concern about one's health) * hysteria |
| officinalis | * insomnia * migraines (does not cause side effects like normal sleeping tablets do, it strengthens the nerves instead of |
| | anesthetizing them) * nervous unrest and nervous tension * neuralgia * neurasthenia * St. Vitus Dance Cardiovascular |
| | Conditions * lowers blood pressure (combined with Mistletoe) * palpitations of the heart * strengthens the heart |
| | Gastrointestinal Conditions * diverticulitis * irritable bowel * nervous dyspepsia * nervous stomach * stimulates digestion * |
| | stomach cramps Female Conditions * menstrual cramps Liver Conditions * aids liver function Respiratory Conditions * head |
| | congestion * helps loosen phlegm in difficult coughs and lung congestion (combined with Anise and Licorice) Other * muscle |
| | spasms * relieves pain Externally can be used for spinal rubs in diseases where the spinal cord needs: * lessened sensibility to |
| | pain * stimulation |
| Vervain - | Brain and Nervous Conditions * depression * fits and convulsion * insomnia * mental stress * migraine and headache caused |
| Verbena | by nerves * nervous disorders * physical and emotional/mental problems that affect the nervous system * restorative remedy |
| officinalis, | for nervous exhaustion and debility * strengthens the nervous system * Mistletoe and Valerian are sometimes combined with |
| Verbena hastata | Vervain for nervous conditions Female Conditions * helps increase the flow of breast milk * painful or irregular menstruation |
| | * post-natal depression * promotes the onset of menstruation Gastrointestinal Conditions * acute diarrhea * benefits the |
| | digestive system * eases pain in the bowels * stomach disorders * stimulates digestion Genitourinary Conditions * bladder |
| | stones * kidney trouble Inflammatory Conditions * reduces inflammation Liver Conditions * biliary tract complaints * |
| | gallstones * jaundice * stimulates liver function * treats liver diseases Metabolic Conditions * stimulates metabolism Parasitic |
| | Conditions * worms in children Respiratory Conditions * asthma * coughs (combined with Horehound) * in early stages of |
| | colds (can be combined with Yarrow) to promote sweating * pleurisy * throat conditions * whooping cough Other * autumn |
| | fever * relieves pain * beneficial in convalescent states * exhaustion and fatigue * in early stages of fevers * intermittent |
| | fevers * ophthalmiarelaxant in cases of muscle tension * ulcers * congestion * dysentery * malaria * coughs and colds * gout |
| | * nerves Externally it is used for: * wounds * ear neuralgia * eczema * headaches * hemorrhoids * rheumatic pain * |
| | rheumatism * skin ailments * tooth decay and gum disease * ulcers of the mouth and gums * weak, sore or inflamed eyes |
| Violet - Viola | Brain and Nervous Conditions * epilepsy * hysterical or nervous complaints * improves a week memory * insomnia |
| odorata | Inflammatory Conditions * eases inflammation * rheumatism Liver Conditions * jaundice Respiratory Conditions * bronchitis |
| | * colds * coughs * dissolving mucous * pleurisy * spasmodic cough with hard breathing * upper respiratory catarrh * |
| | inflamed gums * sore or inflamed throats Other * allays (relieves) pain * heals internal ulcers * cancer in general * cancer of |
| | the tongue * malignant growths * tumors It is used both externally: * abscesses * bronchitis * cancerous growths * pimples * |
| | rheumatism of the wrists * skin irritations * sores * superficial tubercles of the glands of the neck * swollen glands * tumors * |
| | acne * eczema * skin disorders * eases the pain of a headache caused through lack of sleep |
| Vitex Agnes | Female Conditions * acne associated with menstrual cycle * amenorrhea * breast tenderness associated with menstrual cycle * |
| Castus - Vitex | dysmenorrhea (painful menstruation) * endometriosis * female infertility * fibrocystic breast disease * hormonal imbalances. |
| Agnus castus | * menopausal conditions * menorrhagia (heavy menstruation) * menstrual disorders * normalizing the reproductive system * |
| | pregnancy and postpartum support * premenstrual tension including irritability, mood swings, headache * progesterone |
| | deficiency * to suppress libido and inspire chastity (which explains one of its common names chaste tree) Other Conditions * |
| | general weakness of the body * pains in the limbs * paralysis * diseases of the uterus |
| Walnut - Juglans | Female Conditions * all mammary excretions * stops the production of milk in nursing mothers (during weaning etc.) |
| nigra, Juglans | Gastrointestinal Conditions * diarrhea * dysentery Glandular Conditions * scrofulous diseases Respiratory Tract Conditions * |
| regia, Juglans | inflamed tonsils * soreness in the mouth Other * skin diseases * leucorrhea The nut is used for: Brain and Nervous System |
| cinerea | Conditions * calms hysteria Female Conditions * stops vomiting during pregnancy Viral Conditions * herpes Other * |
| | promotes strength * weight gain The ground hull of the nut is used to treat: Viral and Parasitic Conditions * head and body |
| | lice * herpes * internal parasites * liver flukes (combined with Wormwood and Cloves) * worms (use unripe or green hulls) |
| | Other * skin diseases Walnut leaf is used to treat: * boils * eczema * hives * running ulcers * sores * colic (internally) * |
| | dandruffdry hair * gangrene * wounds |
| Wild Cherry - | Gastrointestinal Conditions * dyspepsia Inflammatory Conditions * catarrhal affections Respiratory Conditions * all illnesses |
| Prunus serotina | related to lung congestion, safe to use even for children * bronchitis * consumption * hacking cough * nervous cough * |
| | whooping cough Other * convalescence from fevers |
| Wild Thyme - | Brain and Nervous System Conditions * drunkenness * headaches and nervous conditions (combined with Rosemary) * |
| Thymus | nightmares (taken when going to bed) Even though they are two opposite conditions Wild Thyme has been used for both: * |
| serpyllum | hysteria * lethargy Gastrointestinal Conditions * flatulence * weak digestion Inflammatory Conditions * catarrh Respiratory |
| | Tract Conditions * convulsive coughs * sore throat * whooping cough |
| Wild Yam - | Brain and Nervous System Conditions * neuralgia * soothes the nerves Female Conditions * infertility * morning sickness |
| Discorea villosa | combined with Vitex Agnus Castus and Dandelion * regulation of the female system (menopause and menstrual problems) |
| | Gastrointestinal Conditions * abdominal and intestinal irritation Genitourinary Conditions * pains in the urinary tract |
| | Glandular Conditions * helps the endocrine system Liver Conditions * bilious colic * helps liver function * hepatic |
| | congestion Respiratory Tract Conditions * spasmodic asthma * spasmodic hiccoughs Other * cholera morbus with cramps * |
| | cramps * nausea * spasms * vomiting |
| Willow Bark - | Bacterial and Parasitic Conditions * inhibits and destroys bacteria and other organisms * worms Blood Conditions * internal |
| Salix alba | bleeding It is used both internally and externally for * bleeding wounds * nosebleed * spitting of blood Gastrointestinal |
| | Conditions * chronic diarrhea * chronic dysentery * dyspepsia, with debility of the digestive organs * heartburn * stomach |
| | ailments Genitourinary Conditions * gout * increases the flow of urine Inflammatory Conditions * arthritis * connective tissue |
| | disorders characterized by inflammation * muscular and arthrodial/articular (synovial joint) rheumatism * reduces |
| | inflammation * spondylitis (inflammation of the spine) Other * allays (relieves) pain * convalescence from acute diseases * |
| | prevents vomiting * reduces fevers * inflammation of the gums * to cleanse and heal eyes that are infected or inflamed * |
| | tonsillitis * for burns * for eczema * for offensive smelling perspiration when used with Borax * for pus filled wounds * for |
| | sores * in a footbath for sweaty feet * in skin eruptions |
| Willow Herb - | Genitourinary Conditions * bladder cancer * cystitis * kidney and bladder problems * urinary tract disorders Male Conditions |
| Epilobium | * complaints which often occur after a prostate gland operation, including burning sensation on urination * disorders of the |
| parviflorum | prostate gland combined with Saw Palmetto Berries * hypertrophy of the prostate gland |
| Witch Hazel - | Bacterial and Parasitic Conditions * chronic gonorrheal urethritis * parasites * worms Blood Conditions * relieves the pain of |
| Hamamelis | hemorrhoids (piles) * stops bleeding from internal organs * stops bleeding from the stomach, lungs and nose Female |
| virginiana | Conditions * menorrhagia (excessive menstruation) * weakness after an abortion Gastrointestinal Conditions * bowel |
| | complaints * diarrhea * dysentery * stomach ulcers Respiratory Conditions * bronchitis * coughs * influenza * mucous |
| | discharges * wasting diseases, e.g. tuberculosis Witch Hazel is used externally for: * bleeding gums * conditions of the mouth |
| | * sore throat * a skin cleanser * bleeding wounds * bruises * burning, tired feet * burns * external hemorrhages * hemorrhoids |
| | (as an enema for internal hemorrhoids or ointment for external hemorrhoids) * inflammatory conditions of the skin * insect |
| | bites and stings * ophthalmia (inflammation of the eyes), diluted with warm water * poison ivy * scalds * vaginal infections |
| | varicose veins |
| Withania - | Blood Conditions * anemia Brain and Nervous System Conditions * nervous exhaustion and debility, especially due to stress |
| Withania | * tonic, rather than over-stimulating, for ‘stressed out’ people Inflammatory Conditions * chronic diseases which are |
| somnifera | inflammatory in nature, e.g. connective tissue diseases Male Conditions * impotence due to devitalization Other * |
| | convalescence after acute illness or extreme stress * emaciation, especially in children Good general tonic for disease |
| | prevention in: * athletes * elderly * pregnancy |
| Wormwood - | Brain and Nervous Conditions * cerebral exhaustion (stimulates the cerebral hemispheres, particularly the cortex cerebrum) * |
| Artemisia | cerebral palsy * epilepsy * nervous diseases, such as neurasthenia * gives tone to persons of a highly nervous temperament * |
| absinthium | prevents drunkenness Female Conditions * menstrual cramps * pain during labor * promotes menstruation Gastrointestinal |
| | Conditions * all problems within the digestive system * flatulence (gas/wind) * gastric pain * halitosis (bad breath) * |
| | heartburn * indigestion * lack of appetite Genitourinary Conditions * dropsy * gout Inflammatory Conditions * soothes spinal |
| | irritability Liver Conditions * jaundice * liver and bladder ailments * liver insufficiency (by stimulating the liver and |
| | gallbladder secretions) Parasitic Conditions * expels intestinal worms, especially with St. John's Wort and Centaury * liver |
| | flukes (combined with Walnut and Cloves) Other * debility after sickness or disease Externally for: * bad ankles * bruises * |
| | fallen arches * flat feet * irritations * menstrual disorders of young girls * sprains * swelling of the joints * to improve |
| | circulation * arthritis * bruises * flat feet, fallen arches, bad ankles * neuralgia * rheumatism * sprains |
| Yarrow - Achillea | Bacterial and Viral conditions * in the early stages of measles and other eruptive diseases * venereal disease * vaginal itch |
| millefolium | from vaginosis Blood Conditions * acts on the blood vessels of the pelvis * bleeding hemorrhoids * blood in the urine * builds |
| | up the blood * coagulates blood * heals internal and external wounds * reduces bleeding * stimulates blood renewal with a |
| | beneficial effect on bone marrow * stops bleeding of external wounds * stops bleeding of lungs and stomach Brain and |
| | Nervous System Conditions * neuritis * migraine headache caused by weather changes Cardiovascular Conditions * angina |
| | pectoris * circulatory disorders * vascular spasms Female Complaints * contraceptive * inflammation of the ovaries * |
| | menstrual irregularities especially in young girls * menopausal problems in older women, especially inner restlessness * |
| | menstrual cramps and pain * pelvic troubles * prolapsus of the uterus * stops internal and external bleeding during childbirth |
| | * uterine fibroids * vaginal itch Gastrointestinal Complaints * abdominal disorders * diarrhea * digestive complaints * |
| | dysentery * enteritis * flatulence (gas/wind) * indigestion and heartburn * inflammation of the gastrointestinal tract * lack of |
| | appetite * regulates bowel movements * relaxes the sphincter muscles * sluggish digestion * stomach cramps, with St. John's |
| | Wort Genitourinary Conditions * aids the function of the kidneys * bedwetting * increases flow urine of urine * sluggish |
| | kidneys Glandular Conditions * heals the glandular system Inflammatory Conditions * rheumatic pains * reduces |
| | inflammation Immune Conditions * fights infection Liver Conditions * aids the function of the liver and gallbladder * |
| | gallbladder conditions * liver disorders * stimulates and regulates the liver * stimulates bile flow Muscular Conditions * |
| | Muscle spasms Respiratory System * breaks a cold * bronchitis * colds * head congestion, accompanied by severe pain, |
| | giddiness, nausea, running and weeping eyes, sharp pain the eyes and nose bleeding * influenza, combined with Elder |
| | Flowers and Peppermint * lung cancer, combined with Calamus Root * relieves body weakness caused by colds and |
| | influenza-like conditions Other Conditions * brings down temperature in fevers by promoting perspiration * gum ailments * |
| | obstructed perspiration * prolonged debilitating illness * toothache It is used externally for: * boils * chafed hands * eczema * |
| | greasy skin * opening the pores of the skin * pimples * poor complexion * prevention of baldness * sore nipples * wound |
| | healing * hemorrhoids |
| Yellow Dock - | Blood Conditions * anemia * bleeding of the lungs * cleanses and enriches the blood * for all diseases of the blood * |
| Rumex crispus | hemorrhoids (piles) Gastrointestinal Conditions * dysentery (seeds) * stimulates muscular activity in the intestines * stomach |
| | tonic Glandular Conditions * glandular swellings * scrofula Inflammatory Conditions * rheumatism Liver Conditions * |
| | bilious complaints * detoxifies the liver * jaundice * liver and gallbladder diseases * liver congestion * nourishes the liver |
| | Respiratory Conditions * irritable tickling cough of upper air-tubes Other * for all diseases of the skin * diphtheria * debility |
| | caused by necrosis, cancer, etc. * scurvy * nourishes the spleen It is used externally for: * acne * eczema * psoriasis * |
| | cutaneous eruptions * itching * sores * swellings |
| Ylang-ylang - | relieve high blood pressure * normalize sebum secretion for skin problems. * aphrodisiac * motion sickness |
| Cananga odorata | |
| Zizyphus jujuba | Brain and Nervous System Conditions * irritability * insomnia due to anxiety and nervous exhaustion In anxiety sufferers it: * |
| var spinosa | improves mood * improves performance * decreases sympathetic nervous system symptoms Cardiovascular Conditions * |
| | hypertension * palpitations Other * excessive sweating * night sweats * prolonging survival after burns |

DRUG TYPE

| GENERIC | BROAD | INTERMEDIATE | OPTIMAL | |
|---|---|---|---|---|
| NAME | RANGE | RANGE | RANGE | USED FOR |
| Restylane | 0.001 mg-500 mg/ml | | | Wrinkles, lip enhancement |
| Botox | 1 unit-1000u | 5 unit-500u | 10 unit-100u | Wrinkles. BOTOX ® is indicated for the treatment |
| | | | | of cervical dystonia in adults to decrease |
| | | | | the severity of abnormal head position and |
| | | | | neck pain associated with cervical dystonia. |
| | | | | BOTOX ® is indicated for the treatment |
| | | | | of severe primary axillary hyperhidrosis that is |
| | | | | inadequately managed with topical agents. |
| | | | | BOTOX ® is indicated for the treatment of |
| | | | | strabismus and blepharospasm associated with |
| | | | | dystonia, including benign essential blepharospasm |
| | | | | or VII nerve disorders in patients 12 years |
| | | | | of age and above. |
| Nicotine | 0.001 mg/ml-500 mg/ml | 0.5 mg/ml-300 mg/ml | 5 mg/ml-200 mg/ml | Smoking Withdraw |
| Glycolic | 0.05 mg/ml-60 mg/ | 0.5 mg/ml-30 mg/ | 5 mg/ml-20 mg/ | Acne, Bleaching, Herpes Zoster |
| Acid | 100 ml | 100 ml | 100 ml | |
| Tadalafil/ | 0.05 mg/ml-50 mg/ml | 0.5 mg/ml-30 mg/ml | 5 mg/ml-20 mg/ml | Erectile Dysfunction |
| Cialis | | | | |
| Vardenafil/ | 0.05 mg/ml-50 mg/ml | 0.5 mg/ml-30 mg/ml | 5 mg/ml-20 mg/ml | Erectile Dysfunction |
| Levitra | | | | |
| Sildenafil/ | 0.05 mg/ml-200 mg/ml | 5 mg/ml-150 mg/ml | 10 mg/ml-100 mg/ml | Erectile Dysfunction |
| Viagra | | | | |
| Melatonin | 0.05 mg/ml-50 mg/ml | 0.5 mg/ml-30 mg/ml | 5 mg/ml-20 mg/ml | Insomnia |
| barbiturates | 0.05 mg/ml-50 mg/ml | 0.5 mg/ml-30 mg/ml | 5 mg/ml-20 mg/ml | Sedative/Insomnia |
| with other | | | | |
| medications | | | | |
| amobarbital- | 0.05 mg/ml-50 mg/ml | 0.5 mg/ml-30 mg/ml | 5 mg/ml-20 mg/ml | Sedative/Insomnia |
| secobarbital | | | | |
| secobarbital | 0.05 mg/ml-50 mg/ml | 0.5 mg/ml-30 mg/ml | 5 mg/ml-20 mg/ml | Sedative/Insomnia |
| pentobarbital | 0.05 mg/ml-50 mg/ml | 0.5 mg/ml-30 mg/ml | 5 mg/ml-20 mg/ml | Sedative/Insomnia |
| butabarbital | 0.05 mg/ml-50 mg/ml | 0.5 mg/ml-30 mg/ml | 5 mg/ml-20 mg/ml | Sedative/Insomnia |
| amobarbital | 0.05 mg/ml-50 mg/ml | 0.5 mg/ml-30 mg/ml | 5 mg/ml-20 mg/ml | Insomnia |
| Zolpidem/ | 0.05 mg/ml-50 mg/ml | 0.5 mg/ml-30 mg/ml | 5 mg/ml-20 mg/ml | Insomnia |
| Ambien | | | | |
| Zaleplon/ | 0.05 mg/ml-50 mg/ml | 0.5 mg/ml-30 mg/ml | 5 mg/ml-20 mg/ml | Insomnia |
| Sonata | | | | |
| Eszopiclone/ | 0.05 mg/ml-50 mg/ml | 0.5 mg/ml-30 mg/ml | 5 mg/ml-20 mg/ml | Insomnia |
| Lunesta | | | | |
| Chloral | 0.05 mg/ml-1000 mg/ml | 0.5 mg/ml-750 mg/ml | 5 mg/ml-500 mg/ml | Insomnia |
| Hydrate | | | | |
| Ramelteon/ | 0.05 mg/ml-50 mg/ml | 0.5 mg/ml-30 mg/ml | 5 mg/ml-20 mg/ml | Insomnia |
| Rozerem | | | | |
| Triazolam/ | 0.05 mg/ml-10 mg/ml | 0.5 mg/ml-2 mg/ml | 0.5 mg/ml-1 mg/ml | Insomnia |
| Halcion | | | | |
| Quazepam/ | 0.05 mg/ml-50 mg/ml | 0.5 mg/ml-30 mg/ml | 5 mg/ml-20 mg/ml | Insomnia |
| Doral | | | | |
| Flurazepam/ | 0.05 mg/ml-100 mg/ml | 0.5 mg/ml-60 mg/ml | 5 mg/ml-30 mg/ml | Insomnia |
| Dalmane | | | | |
| Temazepam/ | 0.05 mg/ml-80 mg/ml | 0.5 mg/ml-30 mg/ml | 5 mg/ml-20 mg/ml | Insomnia |
| Restoril | | | | |
| Estazolam/ | 0.05 mg/ml-50 mg/ml | 0.5 mg/ml-30 mg/ml | 5 mg/ml-20 mg/ml | Insomnia |
| Prosom | | | | |
| Oxazepam/ | 0.05 mg/ml-50 mg/ml | 0.5 mg/ml-30 mg/ml | 5 mg/ml-20 mg/ml | Ativan (lorazepam) is indicated for the |
| Serax | | | | management of anxiety disorders or for the short- |
| | | | | term relief of the symptoms of anxiety or |
| | | | | anxiety associated with depressive symptoms. |
| | | | | Anxiety or tension associated with the stress |
| | | | | of everyday life usually does not require |
| | | | | treatment with an anxiolytic. |
| Lorazepam/ | 0.05 mg/ml-50 mg/ml | 0.5 mg/ml-30 mg/ml | 5 mg/ml-20 mg/ml | Ativan (lorazepam) is indicated for the |
| Ativan | | | | management of anxiety disorders or for the short- |
| | | | | term relief of the symptoms of anxiety or |
| | | | | anxiety associated with depressive symptoms. |
| | | | | Anxiety or tension associated with the stress |
| | | | | of everyday life usually does not require |
| | | | | treatment with an anxiolytic. |
| Alprazolam/ | 0.05 mg/ml-50 mg/ml | 0.5 mg/ml-30 mg/ml | 5 mg/ml-20 mg/ml | Anxiety Disorders, Panic Disorder |
| Xanax | | | | |
| Valproic | 0.05 mg/ml-4000 mg/ml | 0.5 mg/ml-3000 mg/ml | 5 mg/ml-2000 mg/ml | DEPAKENE (valproic acid) is indicated |
| acid/ | | | | as monotherapy and adjunctive therapy in the |
| Depakene | | | | treatment of patients with complex |
| | | | | partial seizures that occur either in isolation or in |
| | | | | association with other types of seizures. |
| | | | | DEPAKENE (valproic acid) is indicated for use as |
| | | | | sole and adjunctive therapy in the treatment |
| | | | | of simple and complex absence seizures, and |
| | | | | adjunctively in patients with multiple seizure |
| | | | | types which include absence seizures. |
| Primidone/ | 0.05 mg/ml-600 mg/ml | 0.5 mg/ml-300 mg/ml | 5 mg/ml-200 mg/ml | Mysoline, used alone or concomitantly |
| Mysoline | | | | with other anticonvulsants, are indicated in the |
| | | | | control of grand mal, psychomotor, and |
| | | | | focal epileptic seizures. It may control grand mal |
| | | | | seizures refractory to other anticonvulsant therapy. |
| Phenytoin/ | 0.05 mg/ml-800 mg/ml | 0.5 mg/ml-400 mg/ml | 5 mg/ml-200 mg/ml | Dilantin is indicated for the control of |
| Dilantin | | | | generalized tonic-clonic (grand mal) and complex |
| | | | | partial (psychomotor, temporal lobe) seizures |
| | | | | and prevention and treatment of seizures |
| | | | | occurring during or following neurosurgery. |
| Phenobarbital/ | 0.05 mg/ml-1000 mg/ml | 0.5 mg/ml-800 mg/ml | 5 mg/ml-400 mg/ml | Sedatives: *Hypnotics, for the short- |
| Phenobarbital | | | | term treatment of insomnia, since they appear to lose |
| | | | | their effectiveness for sleep induction and |
| | | | | sleep maintenance after 2 weeks. (See CLINICAL |
| | | | | PHARMACOLOGY.) *Preanesthetics *Long-term |
| | | | | anticonvulsants for the treatment of generalized |
| | | | | tonic-clonic and cortical local seizures. And, |
| | | | | in the emergency control of certain acute |
| | | | | convulsive episodes, e.g., those associated |
| | | | | with status epilepticus, cholera, eclampsia, |
| | | | | meningitis, tetanus, and toxic reactions to |
| | | | | strychnine or local anesthetics. |
| Oxcarbazepine/ | 0.05 mg/ml-1000 mg/ml | 0.5 mg/ml-800 mg/ml | 5 mg/ml-400 mg/ml | Trileptal ® (oxcarbazepine) is |
| Trileptal | | | | indicated for use as monotherapy or adjunctive therapy |
| | | | | in the treatment of partial seizures in adults and as |
| | | | | monotherapy in the treatment of partial seizures |
| | | | | in children aged 4 years and above with epilepsy, |
| | | | | and as adjunctive therapy in children aged |
| | | | | 2 years and above with epilepsy. |
| Levetiracetam/ | 0.05 mg/ml-800 mg/ml | 0.5 mg/ml-500 mg/ml | 5 mg/ml-300 mg/ml | Epilepsy |
| Keppra | | | | |
| Lamotrigine/ | 0.05 mg/ml-500 mg/ml | 0.5 mg/ml-200 mg/ml | 5 mg/ml-100 mg/ml | Bipolar Disease, Epilepsy |
| Lamictal | | | | |
| Gabapentin/ | 0.05 mg/ml-2000 mg/ml | 0.5 mg/ml-800 mg/ml | 5 mg/ml-600 mg/ml | Postherpetic Neuralgia Neurontin |
| Neurontin | | | | (gabapentin) is indicated for the management |
| | | | | of postherpetic neuralgia in |
| | | | | adults *Epilepsy |
| Carbamazepine/ | 0.05 mg/ml-800 mg/ml | 0.5 mg/ml-600 mg/ml | 5 mg/ml-400 mg/ml | Epilepsy: *Partial seizures with complex |
| Tegretol | | | | symptomatology (psychomotor, temporal lobe). |
| | | | | Patients with these seizures appear to show |
| | | | | greater improvement than those with other types |
| | | | | *Generalized tonic-clonic seizures (grand mal) |
| | | | | *Mixed seizure patterns which include the |
| | | | | above, or other partial or generalized seizures. |
| | | | | Absence seizures (petit mal) do not appear to |
| | | | | be controlled by Tegretol (see PRECAUTIONS, |
| | | | | General). *Trigeminal Neuralgia |
| Bupropion/ | 0.05 mg/ml-700 mg/ml | 0.5 mg/ml-400 mg/ml | 5 mg/ml-300 mg/ml | Major Depressive Disorder |
| Wellbutrin | | | | *Seasonal Affective Disorder |
| Mirtazapine/ | 0.05 mg/ml-700 mg/ml | 0.5 mg/ml-400 mg/ml | 5 mg/ml-300 mg/ml | Major Depressive Disorder |
| Remeron | | | | |
| Trazodone/ | 0.05 mg/ml-700 mg/ml | 0.5 mg/ml-400 mg/ml | 5 mg/ml-300 mg/ml | Major Depressive Disorder |
| Desyrel | | | | |
| Nefazodone/ | 0.05 mg/ml-400 mg/ml | 0.5 mg/ml-200 mg/ml | 5 mg/ml-100 mg/ml | Major Depressive Disorder |
| Serzone | | | | |
| Venlafaxine/ | 0.05 mg/ml-300 mg/ml | 0.5 mg/ml-200 mg/ml | 5 mg/ml-100 mg/ml | Major Depressive Disorder *Obessive |
| Effexor | | | | Compulsive Disorder *Panic Disorder |
| | | | | *Social Anxiety Disorder *General Anxiety |
| Duloxetine/ | 0.05 mg/ml-200 mg/ml | 0.5 mg/ml-90 mg/ml | 10 mg/ml-70 mg/ml | Major Depressive Disorder *Diabetic |
| Cymbalta | | | | Peripheral Neuropathic Pain |
| | | | | *Generalised Anxiety Disorder |
| Sertraline/ | 0.05 mg/ml-100 mg/ml | 0.5 mg/ml-60 mg/ml | 5 mg/ml-40 mg/ml | Major Depressive Disorder *Obsessive |
| Zoloft | | | | Compulsive Disorder *Panic Disorder *Social |
| | | | | Anxiety Disorder *General Anxiety *PTSD |
| | | | | *Premenstral Dysphoric Disorder |
| Paroxetine/ | 0.05 mg/ml-100 mg/ml | 0.5 mg/ml-60 mg/ml | 5 mg/ml-40 mg/ml | Major Depressive Disorder *Obsessive |
| Paxil | | | | Compulsive Disorder *Panic Disorder *Social |
| | | | | Anxiety Disorder *General Anxiety *PTSD |
| | | | | *Premenstral Dysphoric Disorder |
| Fluvoxamine/ | 0.05 mg/ml-100 mg/ml | 0.5 mg/ml-60 mg/ml | 5 mg/ml-40 mg/ml | Obsessive Compulsive Disorder |
| Luvox | | | | |
| Fluoxetine/ | 0.05 mg/ml-100 mg/ml | 0.5 mg/ml-60 mg/ml | 5 mg/ml-40 mg/ml | Major Depressive Disorder |
| Prozac | | | | *Obsessive Compulsive Disorder |
| Escitalopram/ | 0.05 mg/ml-80 mg/ml | 0.5 mg/ml-60 mg/ml | 5 mg/ml-40 mg/ml | Lexapro (escitalopram) is indicated |
| Lexapro | | | | for the treatment of major depressive disorder |
| Citalopram/ | 0.05 mg/ml-80 mg/ml | 0.5 mg/ml-60 mg/ml | 5 mg/ml-40 mg/ml | Celexa (citalopram HBr) is indicated for |
| Celexa | | | | the treatment of depression. |
| Trifluoperazine/ | 0.05 mg/ml-400 mg/ml | 0.5 mg/ml-300 mg/ml | 5 mg/ml-200 mg/ml | For the management of schizophrenia |
| Stelazine | | | | *Stelazine (trifluoperazine HCl) is effective for the |
| | | | | short-term treatment of generalized |
| | | | | non-psychotic anxiety. |
| Thiothixene/ | 0.05 mg/ml-400 mg/ml | 0.5 mg/ml-300 mg/ml | 5 mg/ml-200 mg/ml | Navane is effective in the |
| Navane | | | | management of schizophrenia. |
| Thioridazine/ | 0.05 mg/ml-800 mg/ml | 0.5 mg/ml-600 mg/ml | 5 mg/ml-300 mg/ml | Mellaril ® (thioridazine HCl) is |
| Mellaril | | | | indicated for the management of schizophrenic patients |
| | | | | who fail to respond adequately to treatment |
| | | | | with other antipsychotic drugs |
| Promazine/ | 0.05 mg/ml-500 mg/ml | 0.5 mg/ml-300 mg/ml | 5 mg/ml-200 mg/ml | Used as an adjunct for short |
| Sparine | | | | term treatment of moderate and severe |
| | | | | psychomotor agitation. Also used to treat |
| | | | | agitation or restlessness in the elderly. |
| Perphenazine/ | 0.05 mg/ml-500 mg/ml | 0.5 mg/ml-300 mg/ml | 5 mg/ml-200 mg/ml | Perphenazine is indicated for use in the |
| Trilafon | | | | management of the manifestations of psychotic |
| | | | | disorders; and for the control of severe |
| | | | | nausea and vomiting in adults. |
| Molindone/ | 0.05 mg/ml-500 mg/ml | 0.5 mg/ml-300 mg/ml | 5 mg/ml-200 mg/ml | MOBAN is indicated for the |
| Moban | | | | management of schizophrenia. The efficacy of MOBAN |
| | | | | in schizophrenia was established in clinical |
| | | | | studies which enrolled newly hospitalized and |
| | | | | chronically hospitalized, acutely ill, schizophrenic |
| | | | | patients as subjects. |
| Mesoridazine/ | 0.05 mg/ml-300 mg/ml | 0.5 mg/ml-200 mg/ml | 5 mg/ml-100 mg/ml | Serentil^{#} (mesoridazine besylate) |
| Serentil | | | | is indicated for the management of schizophrenic |
| | | | | patients who fail to respond adequately to |
| | | | | treatment with other antiosychotic drugs. |
| | | | | Due to the risk of significant. potentially life- |
| | | | | threatening, proarrhythmic effects with Serentil |
| | | | | treatment. |
| | | | | Serentil should be used only in patients who have |
| | | | | failed to respond adequately to treatment |
| | | | | with appropriate courses of other antipsychotic drugs, |
| | | | | either because of insufficient effectiveness or |
| | | | | the inability to achieve an effective dose due |
| | | | | to intolerable adverse effects from those drugs. |
| | | | | Consequently, before intiating treatment with |
| | | | | Serentil, it is strongly recommended that a |
| | | | | patient be given at least two trials, each with |
| | | | | a different antipsychotic drug product at an |
| | | | | adequate dose, and for an adequate duration |
| Loxapine/ | 0.05 mg/ml-300 mg/ml | 0.5 mg/ml-200 mg/ml | 5 mg/ml-100 mg/ml | Loxapine Capsules USP are |
| Loxitane | | | | indicated for the treatment of schizophrenia. |
| | | | | The efficacy of loxapine in schizophrenia was |
| | | | | established in clinical studies which enrolled |
| | | | | newly hospitalized and chronically hospitalized |
| | | | | acutely ill schizophrenic patients |
| Haloperidol/ | 0.05 mg/ml-150 mg/ml | 0.5 mg/ml-100 mg/ml | 5 mg/ml-40 mg/ml | HALDOL (haloperidol) is indicated |
| Haldol | | | | for use in the treatment of schizophrenia *HALDOL is |
| | | | | indicated for the control of tics and vocal |
| | | | | utterances of Tourette's Disorder. |
| Fluphenazine/ | 0.05 mg/ml-300 mg/ml | 0.5 mg/ml-200 mg/ml | 5 mg/ml-100 mg/ml | Fluphenazine hydrochloride is indicated |
| Prolixin | | | | in the management of manifestations of psychotic |
| | | | | disorders *Fluphenazine hydrochloride has not |
| | | | | been shown effective in the management of |
| | | | | behaviorial complications in patients with |
| | | | | mental retardation. |
| Chlorpromazine/ | 10 mg/ml-1500 mg/ml | 50 mg/ml-1000 mg/ml | 100 mg/ml-600 mg/ml | For the treatment of schizophrenia |
| Thorazine | | | | *To control nausea and vomiting *For relief of |
| | | | | restlessness and apprehension before |
| | | | | surgery *For acute intermittent porphyria *As |
| | | | | an adjunct in the treatment of tetanus *To |
| | | | | control the manifestations of the manic type |
| | | | | of manic-depressive illness |
| Ziprasidone/ | 0.05 mg/ml-400 mg/ml | 0.5 mg/ml-300 mg/ml | 5 mg/ml-200 mg/ml | Schizopherenia/Bi-Polar |
| Geodon | | | | |
| Risperidone/ | 0.05 mg/ml-50 mg/ml | 0.5 mg/ml-30 mg/ml | 5 mg/ml-15 mg/ml | Schizopherenia/Bi-Polar |
| Risperdal | | | | |
| Paliperidone/ | 0.05 mg/ml-500 mg/ml | 0.5 mg/ml-300 mg/ml | 5 mg/ml-100 mg/ml | Schizopherenia/Bi-Polar |
| Invega | | | | |
| Quetiapine/ | 0.05 mg/ml-600 mg/ml | 0.5 mg/ml-400 mg/ml | 5 mg/ml-200 mg/ml | Schizopherenia/Bi-Polar |
| Seroquel | | | | |
| Olanzapine/ | 0.05 mg/ml-100 mg/ml | 0.5 mg/ml-50 mg/ml | 5 mg/ml-25 mg/ml | Schizopherenia |
| Zyprexa | | | | |
| Clozapine/ | 0.05 mg/ml-800 mg/ml | 0.5 mg/ml-60 mg/ml | 100 mg/ml-500 mg/ml | Schizopherenia/Bi-Polar |
| Clozaril | | | | |
| Aripiprazole/ | 0.05 mg/ml-100 mg/ml | 0.5 mg/ml-75 mg/ml | 5 mg/ml-40 mg/ml | Depression/Bi-Polar-Disordered, |
| Abilify | | | | Manic, Mixed/Schizopherenia |
| Tranylcypromine/ | 0.05 mg/ml-200 mg/ml | 0.5 mg/ml-100 mg/ml | 5 mg/ml-50 mg/ml | Depression |
| Parnate | | | | |
| Phenelzine/ | 0.05 mg/ml-200 mg/ml | 0.5 mg/ml-100 mg/ml | 5 mg/ml-50 mg/ml | Depression |
| Nardil | | | | |
| Isocarboxazid/ | 0.05 mg/ml-200 mg/ml | 0.5 mg/ml-100 mg/ml | 5 mg/ml-50 mg/ml | Depression |
| Marplan | | | | |
| amitripytline | 0.05 mg/ml-30 mg/ml | 0.5 mg/ml-20 mg/ml | 5 mg/ml-10 mg/ml | Depression |
| and | | | | |
| perphenazine/ | | | | |
| Etrafon | | | | |
| Doxepin/ | 0.05 mg/ml-400 mg/ml | 0.5 mg/ml-200 mg/ml | 5 mg/ml-150 mg/ml | Depression |
| Sinequam | | | | |
| Amoxapine/ | 0.05 mg/ml-300 mg/ml | 0.5 mg/ml-150 mg/ml | 5 mg/ml-100 mg/ml | Depression |
| Amoxapine | | | | |
| Amitriptyline/ | 0.05 mg/ml-250 mg/ml | 0.5 mg/ml-150 mg/ml | 25 mg/ml-100 mg/ml | Depression *Pain management |
| Elavil | | | | |
| Serotonin | 0.05 mg/ml-200 mg/ml | 0.5 mg/ml-100 mg/ml | 5 mg/ml-60 mg/ml | Depression |
| HCL | | | | |

| GENERIC | | BROAD | INTERMEDIATE | | |
|---|---|---|---|---|---|
| NAME | COMPOSED OF | RANGE | RANGE | OPTIMAL RANGE | Used for: |
| PAIN OFF | Baclofen (Liorisel) | 1 mg-300 mg | 5 mg-200 mg | 10 mg-100 mg | Pain Management |
| | Ketoprofen (Orudis) | 1 mg-300 mg | 5 mg-200 mg | 10 mg-100 mg | |
| | Lidocaine | 0.01 mg-100 mg | 1 mg-70 mg | 5 mg-50 mg | |
| | Gabapentin (Neurontin) | 100 mg-3000 mg | 300 mg-2000 mg | 500 mg-1500 mg | |

1. A treatment device for treating a medical condition, the device comprising:
a composition selected for its ability to treat the medical condition, the composition being formulated for transdermal delivery;
a portable container comprising a body for accommodating the composition in a sealed arrangement, a frangible seal on the container which can be ruptured to enable discharge of the composition from the body, and an application component on the container for facilitating transdermal delivery of the composition.

2. A treatment device as claimed in claim 1 wherein the composition is diazepam.

3. A treatment device as claimed in claim 1 wherein the composition is selected from one of the following: Botox®, Viagra®, Cialis®, Levitra®, Nicotine, and Glycolic Acid.

4. A treatment device as claimed in claim 1 wherein the composition is selected from one of the generic name substances as set forth in Table I of the attached description.

5. A treatment device as claimed in claim 1 wherein the portable container comprises a finger ring.

6. A treatment device as claimed in claim 1 wherein the portable container comprises a necklace.

7. A treatment device as claimed in claim 1 wherein the portable container comprises a bracelet.

8. A treatment device as claimed in claim 1 wherein the portable container comprises a bandage.

9. A treatment device as claimed in claim 1 wherein the application component on the container comprises a vibrator.

10. A treatment device as claimed in claim 9 wherein the vibrator is a vibrating capsule have a power source and switch for activating the vibrator.

11. A treatment device as claimed in claim 5 wherein the finger ring comprises a band portion worn around the finger, a chamber for accommodating the composition, and chamber for a vibrator.

12. A treatment device as claimed in claim 1 further comprising a latex sheath to be worn by a person facilitating transdermal delivery to prevent any portion of the composition from adversely affecting the person facilitating transdermal delivery.

13. A treatment device as claimed in claim 1 wherein the composition comprises and active ingredient for treating the medical condition, the active ingredient being present as a dosage of about 0.05 mg/ml-50 mg/ml.

14. A treatment device as claimed in claim 13 where the active ingredient is present in the amount of about 0.5 mg/ml-30 mg/ml.

15. A treatment device as claimed in claim 14 wherein the active ingredient is present in the amount of about 5 mg/ml-20 mg/ml.

16. A treatment device as claimed in claim 1 wherein the composition further comprises liposome cream as a carrier and a penetration enhancer.

17. A treatment device as claimed in claim 1 wherein the composition further comprises DMSO (Dimethyl Sulfoxide) in the amount of about 0.05% to about 90% for skin penetration enhancement.

18. A treatment device as claimed in claim 1 wherein the composition further comprises a pluronic lecithin organogel (PLO) base as a carrier and a penetration enhancer.

19. A method of treating a medical condition comprising:
providing a composition selected for its ability to treat the medical condition, and formulating the composition for transdermal delivery;
placing the composition in a sealed chamber of a portable container;
rupturing a frangible seal of the chamber when the composition is needed for treatment to enable discharge of the composition from the body;
applying the composition to an accessible part of a person being treated with the composition; and
facilitating the application of the composition to the person by utilizing an application component formed on the container to enhance transdermal delivery of the composition to the person.

20. A method as claimed in claim 19 wherein the application is facilitated by a vibrator to improve rapid transdermal delivery of the composition to the person.

21. A method as claimed in claim 19 further comprising the use of a latex sheath to protect by the person applying the composition.

22. A method as claimed in claim 19 wherein the composition is applied to the person behind or below the ear or temple.

23. A method as claimed in claim 19 wherein the composition is applied to the person on any part of the body best suited for treating the medical condition.

25. A method of treating seizure comprising applying Diestat® gel above the shoulder.

26. A method of treating erectile dysfunction comprising applying a composition selected from the group consisting of Viagra®/Sildenafil, Levitra®/Vardenafil and Cialis/Tadalafil to the penis.

27. A method of treating the face and neck comprising applying Botox to the face and neck.
